(19) United States
(12) Patent Application Publication
(10) Pub. No.: US 2025/0161347 A1
(43) Pub. Date: May 22, 2025

(54) METHODS AND COMPOSITIONS FOR TREATING NON-ALCOHOLIC FATTY LIVER DISEASE

(71) Applicant: Flagship Pioneering Innovations VII, LLC, Cambridge, MA (US)

(72) Inventors: Katharine Maryell VON HERRMANN, Lexington, MA (US); Zehua CHEN, Cambridge, MA (US); Jonathan Michel Yohan FILLATRE, Boston, MA (US); Jordan Otto JAGIELSKI, Boston, MA (US); Anna RUSNAK, Somerville, MA (US); Artem SOKOLOV, Boston, MA (US); Sachit Dinesh SAKSENA, Boston, MA (US); Baixue YAO, Cambridge, MA (US); Wenyi ZENG, Allston, MA (US); Scott Logan LIPNICK, Boston, MA (US); Torben Lauesgaard STRAIGHT NISSEN, Chestnut Hill, MA (US)

(21) Appl. No.: 18/956,705

(22) Filed: Nov. 22, 2024

Related U.S. Application Data
(60) Provisional application No. 63/553,202, filed on Feb. 14, 2024.
(60) Provisional application No. 63/602,236, filed on Nov. 22, 2023.

(51) Int. Cl. A61K 31/713; A61P 1/16

(52) U.S. Cl. CPC A61K 31/713; A61P 1/16

(57) ABSTRACT

Disclosed herein are compositions (e.g., inhibitory agents, such as inhibitory nucleic acid molecules) and methods for treating subjects having or at risk of developing non-alcoholic fatty liver disease (NAFLD), such as non-alcoholic fatty liver (NAFL) or non-alcoholic steatohepatitis (NASH). Such methods can slow, inhibit, or even reverse further progression of NAFLD.

### SEQUENCE LISTING

The instant application contains a Sequence Listing which has been submitted electronically in XML format and is hereby incorporated by reference in its entirety. Said XML copy, created on Nov. 14, 2024, is named “51595-004002_Sequence_Listing_11_14_24” and is 219,773 bytes in size.

### FIELD OF THE INVENTION

The present disclosure relates to the treatment of nonalcoholic fatty liver disease, such as nonalcoholic fatty liver or nonalcoholic steatohepatitis.

### BACKGROUND

Nonalcoholic fatty liver disease (NAFLD) (also referred to as steatotic liver disease (SLD), metabolic dysfunction-associated fatty liver disease (MAFLD), or metabolic dysfunction-associated steatotic liver disease (MASLD)) is a growing health concern that affects approximately one-third of adults and a growing population of children in developed countries. Early stages of the disease are characterized by an accumulation of lipids, e.g., as lipid droplets, in the cytoplasm of hepatocytes. Hepatic steatosis is defined as the presence of lipid droplets in more than 5% of hepatocytes and is the main criteria for diagnosing NAFLD, such as nonalcoholic fatty liver (NAFL). In some instances, hepatic steatosis may be reversed with proper diet and exercise, thereby reversing NAFL. However, in instances where lipids (e.g., triglycerides) continue to accumulate in hepatocytes, NAFL can progress to nonalcoholic steatohepatitis (NASH), a condition further characterized by hepatocyte injury, liver inflammation, and potentially fibrosis. Within a decade of developing NASH, patients with NASH may develop cirrhosis, wherein injured and dying hepatocytes are replaced by scar tissue in later stages of fibrosis. Current treatment options include dietary changes, losing weight, undergoing bariatric surgery, and treating comorbidities. For individuals with advanced liver inflammation, steatosis, and/or fibrosis, a liver transplant may be the only viable treatment. Thus, there remains a need for therapeutic interventions to treat NAFLD (e.g., NAFL and NASH).

### SUMMARY OF THE INVENTION

In one aspect, the invention features a method of treating a human subject identified as having nonalcoholic fatty liver disease (NAFLD), the method including the step of administering to the subject an inhibitor (e.g., an effective amount of an inhibitor) of a target listed in Table 1.

In some embodiments, the target listed in Table 1 is ATP binding cassette subfamily B member 4 (ABCB4). In some embodiments, the target listed in Table 1 is complement C8 beta chain (C8B). In some embodiments, the target listed in Table 1 is homogentisate 1,2-dioxygenase (HGD). In some embodiments, the target listed in Table 1 is methylmalonyl-CoA epimerase (MCEE). In some embodiments, the target listed in Table 1 is SH3 and PX domains 2A (SH3PXD2A). In some embodiments, the target listed in Table 1 is solute carrier family 16 member 10 (SLC16A10). In some embodiments, the target listed in Table 1 is transthyretin (TTR). In some embodiments, the target listed in Table 1 is haptoglobin-related protein (HPR). In some embodiments, the target listed in Table 1 is peroxisomal biogenesis factor 6 (PEX6). In some embodiments, the target listed in Table 1 is RAB11A, Member RAS Oncogene Family (RAB11A). In some embodiments, the target listed in Table 1 is solute carrier family 22 member 25 (SLC22A25).

In some embodiments, the subject has >5% liver steatosis. In some embodiments, the subject has ≤5% liver steatosis.

In some embodiments, the NAFLD is nonalcoholic fatty liver (NAFL) or nonalcoholic steatohepatitis (NASH).

In some embodiments, the inhibitor is delivered to a hepatocyte (HC) in the subject.

In another aspect, the invention features a method of treating a human subject identified as having nonalcoholic fatty liver disease (NAFLD), the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In some embodiments, the subject has >5% liver steatosis. In some embodiments, the subject has ≤5% liver steatosis.

In some embodiments, the NAFLD is NAFL or NASH.

In some embodiments, the inhibitor is delivered to a HC in the subject.

In another aspect, the invention features a method of treating a human subject identified as at risk of developing NAFLD, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In some embodiments, the subject has >5% liver steatosis. In some embodiments, the subject has ≤5% liver steatosis.

In some embodiments, the NAFLD is NAFL or NASH.

In some embodiments, the inhibitor is delivered to a HC in the subject.

In another aspect, the invention features a method of treating liver steatosis in a human subject in need thereof, the method including the step of administering to the subject an inhibitor (e.g., an effective amount of an inhibitor) of a target listed in Table 1.

In another aspect, the invention features a method of treating liver inflammation in a human subject in need thereof, the method including the step of administering to the subject an inhibitor (e.g., an effective amount of an inhibitor) of a target listed in Table 1.

In another aspect, the invention features a method of treating liver fibrosis in a human subject in need thereof, the method including the step of administering to the subject an inhibitor (e.g., an effective amount of an inhibitor) of a target listed in Table 1.

In another aspect, the invention features a method of reducing the number of lipid droplets in a liver cell of a human subject, the method including the step of administering to the subject an inhibitor (e.g., an effective amount of an inhibitor) of a target listed in Table 1.

In another aspect, the invention features a method of reducing lipid droplet area in a liver cell of a human subject, the method including the step of administering to the subject an inhibitor (e.g., an effective amount of an inhibitor) of a target listed in Table 1.

In some embodiments of any of the foregoing aspects, the target listed in Table 1 is ABCB4. In some embodiments of any of the foregoing aspects, the target listed in Table 1 is C8B. In some embodiments of any of the foregoing aspects, the target listed in Table 1 is HGD. In some embodiments of any of the foregoing aspects, the target listed in Table 1 is MCEE. In some embodiments of any of the foregoing aspects, the target listed in Table 1 is SH3PXD2A. In some embodiments of any of the foregoing aspects, the target listed in Table 1 is SLC16A10. In some embodiments of any of the foregoing aspects, the target listed in Table 1 is TTR. In some embodiments of any of the foregoing aspects, the target listed in Table 1 is HPR. In some embodiments of any of the foregoing aspects, the target listed in Table 1 is PEX6. In some embodiments of any of the foregoing aspects, the target listed in Table 1 is RAB11A. In some embodiments of any of the foregoing aspects, the target listed in Table 1 is SLC22A25.

In another aspect, the invention features a method of treating liver steatosis in a human subject in need thereof, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In another aspect, the invention features a method of treating liver inflammation in a human subject in need thereof, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In another aspect, the invention features a method of treating liver fibrosis in a human subject in need thereof, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In another aspect, the invention features a method of reducing the number of lipid droplets in a liver cell of a human subject, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In another aspect, the invention features a method of reducing lipid droplet area in a liver cell of a human subject, the method including the step of administering to the subject an inhibitor (e.g., an effective amount of an inhibitor) of ABCB4, C8B, HGD, MCEE, SH3PXD2A, SLC16A10, TTR, HPR, PEX6, RAB11A, and/or SLC22A25.

In some embodiments of any of the foregoing aspects, the liver cell is a hepatocyte.

In some embodiments of any of the foregoing aspects, the inhibitor is an inhibitory nucleic acid molecule against the target, e.g., an siRNA that reduces expression or activity of the target by 10% or more (e.g., 10%, 20%, 30%, 40%, 50%, 60%, 70%, 80%, 90%, 95%, 98%, 99% or more).

In some embodiments of any of the foregoing aspects, the inhibitor is an agent listed in Table 2.

In some embodiments, the inhibitor is selected from the group consisting of: (i) a small interfering RNA (siRNA) (e.g., an siRNA listed in Table 3 or Table 4); (ii) an antisense oligonucleotide (ASO) (e.g., an ASO listed in Table 3); (iii) a small molecule; (iv) an antibody (e.g., an antibody listed in Table 3); and (v) a protein or a peptide (e.g., a protein or peptide listed in Table 3).

In some embodiments of any of the foregoing aspects, the method further comprises the step of administering to the subject an additional therapeutic agent. In some embodiments, the additional therapeutic agent is selected from the group consisting of: (i) an siRNA (e.g., an siRNA listed in Table 3 or Table 4); (ii) an ASO (e.g., an ASO listed in Table 3); (iii) a small molecule (e.g., a small molecule listed in Table 3); (iv) an antibody (e.g., an antibody listed in Table 3); and (v) a protein or a peptide (e.g., a protein or peptide listed in Table 3).

In some embodiments of any of the foregoing aspects, the inhibitor is administered parenterally. In some embodiments, the inhibitor is administered in an amount sufficient to reduce liver steatosis, liver inflammation, and/or liver fibrosis in the subject. In some embodiments, the inhibitor includes a delivery vehicle (e.g., a lipid nanoparticle (LNP)).

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10% in either direction (greater than or less than) of a stated reference value, unless otherwise stated or otherwise evident from the context (except where such number would exceed 100% of a possible value).

As used herein, “administration” refers to providing or giving a subject an agent (e.g., an agent listed in Table 2) by any effective route. Exemplary routes of administration are described herein below.

As used herein, the term “antagonist” refers to an agent (e.g., an antibody, small molecule, or soluble protein) that reduces or inhibits the activity of a target molecule (e.g., a target in Table 1) through directly binding to or directly modulating the target molecule. For example, in embodiments in which the target molecule is a receptor, an antagonist may reduce receptor activity by directly binding to the receptor, by blocking the receptor binding site, or by modulating receptor conformation (e.g., maintaining a receptor in a closed or inactive state). An antagonist reduces the activity of a target molecule by 10%, 20%, 30%, 40%, 50%, 60%, 70%, 80%, 90%, or more.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)₂ fragments) that are capable of specifically binding to a target protein. Fab and F(ab′)₂ fragments lack the Fc fragment of an intact antibody.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature 341:544-546, 1989) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “at risk of developing” in the context of a disease or condition (e.g., NAFLD, NAFL, or NASH) refers to a state of a human subject that has an increased probability or likelihood of developing the disease or condition in their lifetime. For example, a subject has an increased risk of developing NAFLD (e.g., NAFL or NASH) if one or more (e.g., 1, 2, 3, 4, 5, 6, or more) of the following features are present: the subject has a metabolic or hormonal disorder (e.g., type 2 diabetes, insulin resistance, hypothyroidism, hypopituitarism, polycystic ovarian syndrome, growth hormone deficiency, hyperuricemia, or dyslipidemia); the subject has hypertension; the subject has gout; the subject has a removed gallbladder; the subject has completed menopause; the subject has an increased level of retinol binding protein 4 (RBP4) (e.g., greater than 1.7 μg/mL), leptin (LEP) (e.g., greater than 24 ng/mL), resistin (RETN) (e.g., greater than 23 ng/mL), or ghrelin (GHRL) (e.g., greater than 170 fmol/mL) or a decreased level of adiponectin (ADIPOQ) (e.g., lower than 7 μg/mL); the subject has an altered liver transaminase level (e.g., alanine transaminase (ALT) greater than about 25 U/L), such as an elevated or altered AST/ALT ratio (e.g., less than 1 or greater than or equal to 2); the subject has an altered cholesterol level, such as high circulating levels of free fatty acids (e.g., about 1 mmol/L or higher), high circulating levels of low-density lipoprotein (LDL)-cholesterol (e.g., 1200 mg/L or higher), or low circulating levels of high-density lipoprotein (HDL)-cholesterol (e.g., 500 mg/L or lower); the subject has a high triglyceride level (e.g., greater than 150 mg/dL); the subject has a high uric acid level (e.g., >4.75 mg/dL); the subject is taking amiodarone (CORDARONE®), diltiazem (CARDIZEM®), tamoxifen (NOLVADEX®), or steroids; the subject has a poor diet, poor exercise habits, sleep deprivation, or obstructive sleep apnea; the subject experiences symptoms of fatigue, loss of appetite, weight loss, or jaundice; the subject has a body mass index (BMI) ≥30 kg/m²; the subject has a large waist circumference (e.g., about 280 cm for males and about 278 cm for females), wherein a large proportion of body fat is located in the abdomen; and other risk factors disclosed in Parameswaran et aL., Cureus, 13(12):e20776, 2021, which is incorporated herein by reference and described herein. The more of these factors the patient has, the greater “the risk of developing” the patient has. While subjects identified as “at risk of developing NAFL” are not clinically diagnosed with NAFLD, they may have additional signs of NAFLD—apart from those mentioned above—as determined by, e.g., a biopsy of one or more tissues (e.g., from the liver) to detect or measure steatosis, a magnetic resonance imaging (MRI) to detect or measure steatosis, a magnetic resonance elastography (MRE) to detect or measure fibrosis, or an ultrasound (e.g., FIBROSCAN®) to detect liver steatosis and/or fibrosis. Subjects “at risk of developing NASH” may have been previously diagnosed with NAFL. Diagnosis of NAFL may be based on diagnostic methods known in the art, including, e.g., a biopsy of one or more tissues (e.g., from the liver), magnetic resonance imaging (MRI) to detect or measure steatosis, magnetic resonance elastography (MRE) to detect or measure fibrosis, or an ultrasound (e.g., FIBROSCAN®) to detect steatosis and/or fibrosis.

As used herein, the term “biological sample” refers to a sample obtained from any one or more cells, tissues (e.g., a biopsy), organs, or extracellular fluids in a subject. A biological sample may be obtained, for example, from a subject's blood, plasma, serum, liver, kidney, lung, skin, muscle, heart, brain, saliva, mucus, hepatocytes, biliary epithelial cells, cholangiocytes, stellate cells, Kupffer cells, macrophages, liver sinusoidal endothelial cells, red blood cells, white blood cells, parietal epithelial cells, epithelial cells, cardiomyocytes, smooth muscle cells, neurons, glial cells, osteoblasts, adipocytes, ova, and/or spermatozoa.

As used herein, the terms “effective amount” of an agent or composition described herein refer to a quantity sufficient to (when administered to the subject (e.g., in vivo, e.g., a human) or a cell of the subject (e.g., ex vivo)) effect beneficial or desired results, including clinical results, and, as such, an “effective amount” or synonym thereto depends upon the context in which it is being applied. For example, in the context of treating NAFLD (e.g., NAFL or NASH), it is an amount of the agent or composition described herein that is sufficient to achieve a treatment response as compared to the response obtained without administration of the composition. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition of the present disclosure is an amount which results in a beneficial or desired result in a subject as compared to a control. As defined herein, a therapeutically effective amount of a composition of the present disclosure may be readily determined by one of ordinary skill by routine methods known in the art. Dosage regimen may be adjusted to provide the optimum therapeutic response.

As used herein, an “experimental sample” refers to a sample (e.g., a biological sample or a clinical image) that is to be compared to a reference sample. The experimental sample may be obtained from a subject that is at risk of developing NAFLD (e.g., developing NAFL or NASH), at risk of NAFLD progression (e.g., developing NASH), and/or already has NAFLD (e.g., NAFL or NASH).

As used herein, the term “inhibitor” refers to an agent that inhibits or reduces gene expression, protein activity, and/or signal transduction of a target (e.g., a target in Table 1). Inhibitors include antagonists that interact directly with the target molecule and other therapeutic agents that decrease the expression, activity, or signaling of the target molecule by interacting with binding partners of the target molecule, upstream regulators of the target molecule, or molecules that mediate signal transduction downstream of the target molecule. Exemplary inhibitors include inhibitory nucleic acid molecules (e.g., a small interfering RNA (siRNA), a double-stranded RNA (dsRNA), a short hairpin RNA (shRNA), a microRNA (miRNA), an antisense oligonucleotide (ASO), or a gapmer); inhibitory antibodies; soluble proteins (e.g., proteins that disrupt the activity of the target, such as dominant negative proteins corresponding to the target or to one of its binding partners); RNA molecules (e.g., mRNA) that encode an inhibitory protein that can disrupt target molecule function; small molecule inhibitors; and programmable nucleases for editing and inhibiting gene expression (e.g., CRISPR-Cas endonucleases programmed to cleave DNA or RNA encoding the target). Inhibitors of a particular target can disrupt the expression, function, and/or signal transduction of the target by 10% or more (e.g., 10%, 20%, 30%, 40%, 50%, 60%, 70%, 80%, 90%, 95%, 98% or more).

As used herein, the term “inhibits” refers to the disruption of a target's (e.g., see Table 1) gene expression, protein expression, function, and/or signal transduction by at least 10% (e.g., 10%, 20%, 30%, 40%, 50%, 60%, 70%, 80%, 90%, 91%, 92%, 93%, 94%, 95%, 96%, 97%, 98% 99%, or 100%).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein “modified” or “modified nucleic acid molecule” refers to a changed state or structure of a nucleic acid molecule described herein. Molecules may be modified in many ways including chemically, structurally, and functionally. In one embodiment, the inhibitory nucleic acid molecules of the present invention are modified by the introduction of non-natural nucleosides and/or nucleotides. In other embodiments, the inhibitory nucleic acid molecules of the present invention are modified by conjugation of an auxiliary moiety. In other embodiments, the RNA molecules (e.g., an mRNA molecule encoding a protein) of the present invention are modified by the introduction of non-natural nucleosides and/or nucleotides. A modified nucleic acid molecule is presented in the disclosure as having the nucleotides adenine (A), thymine (T), cytosine (C), and guanine (G). A thymine may be understood to be a uracil (U) where appropriate, i.e., when the given nucleic acid is an RNA molecule.

As used herein, the term “inhibitory nucleic acid molecule” in the context of a target (e.g., a protein of Table 1) refers to a nucleic acid molecule that has sufficient complementarity to bind to a nucleic acid encoding the target (e.g., has sufficient complementarity to bind to the sequence of a transcript (e.g., a gene or splice variant thereof) listed in Table 1) and inhibit or reduce expression, function, or activity of the target. Exemplary inhibitory nucleic acid molecules are siRNAs, dsRNAs, miRNAs, shRNAs, ASOs, and gapmers. Inhibitory nucleic acid molecules may reduce expression or activity of a target by 10% or more (e.g., 10%, 20%, 30%, 40%, 50%, 60%, 70%, 80%, 90%, 95%, 98%, 99% or more). An inhibitory nucleic acid molecule is presented in the disclosure as having the nucleotides adenine (A), thymine (T), cytosine (C), and guanine (G). A thymine may be understood to be a uracil (U) where appropriate, i.e., when the given nucleic acid is an RNA molecule.

As used herein, the term “pharmaceutical composition” refers to a mixture containing an agent, optionally in combination with one or more pharmaceutically acceptable excipients, diluents, and/or carriers, to be administered to a subject.

As used herein, the term “pharmaceutically acceptable” refers to those compounds, materials, compositions, and/or dosage forms which are, within the scope of sound medical judgment, suitable for use in contact with the tissues a subject, such as a mammal (e.g., a human) without excessive toxicity, irritation, allergic response, or other problem or complication, commensurate with a reasonable benefit/risk ratio.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the terms “polynucleotide” and “nucleic acid molecule” refer to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence,” as used herein, refers to the polynucleotide material itself or to the sequence information (i.e., the succession of letters used as abbreviations for bases) that biochemically characterizes a specific nucleic acid. A polynucleotide sequence presented herein is presented in a 5′ to 3′ direction unless otherwise indicated. A polynucleotide is presented in the disclosure as having the nucleotides adenine (A), thymine (T), cytosine (C), and guanine (G). A thymine may be understood to be a uracil (U) where appropriate, i.e., when the given nucleic acid is an RNA molecule.

As used herein, the terms “reference,” “reference sample,” and “control sample” are used interchangeably to describe a predetermined reference value (e.g., a diagnostic reference level) of a sample (e.g., a biological sample or a clinical image) obtained from a subject. In some instances, the reference is obtained from a subject who has been determined to not have NAFLD (e.g., NAFL and/or NASH) and/or has a value of liver steatosis below 5% and a BMI below 30 kg/m². In some instances, the reference is obtained from a subject that that has or exhibits signs and symptoms of NAFLD prior to undergoing the methods of treatment described herein. In this instance, the reference serves as an initial reference value for a future value (e.g., from an experimental sample) to be compared to. The reference may be derived from a sample, such as a biological sample (e.g., a biopsy) or clinical image (e.g., an image from an MRI, MRE, or ultrasound). The reference may be obtained from a reference value or set of reference values in a given reference chart (e.g., one or more MRE values, ultrasound values, biomarker levels, or histopathological values). A reference should be obtained from the same sample type (e.g., blood, plasma, serum, liver, kidney, muscle, etc.) as an experimental sample. A reference will be used as a reference point to determine the efficacy of the methods of treatment described herein.

As used herein, the terms “relative to a reference,” “relative to a reference sample,” and “relative to a control sample” are used interchangeably to describe a comparison between an experimental sample and a reference sample, e.g., a reference value or reference. For example, use of the following formula constitutes a comparison (e.g., fold-change) between an experimental sample and a reference and can be used to determine the efficacy of treatment.

$$Fold Change = \frac{Experimental Sample}{Reference}$$

As used herein, the term “sample” refers to an organ (e.g., a liver), a subset of its tissues (e.g., a biopsy), cells or component parts (e.g., body fluids, including but not limited to peripheral blood, serum, plasma, ascites, urine, saliva, and hair), a homogenate, a lysate or extract prepared from an organism or a subset of its tissues, cells or component parts, or an image (e.g., a clinical image, such as an MRI or an ultrasound, or histopathological image, such as histological stain) of an organ (e.g., a liver), tissue (e.g., a biopsy), or cells (e.g., hepatocytes). A sample further refers to a medium, such as a nutrient broth or gel, which may contain cellular components, such as proteins or nucleic acid molecules.

As used herein, the term “standard of care” refers to a treatment regimen or process for a particular disease (e.g., NAFLD) that would be recommended by a clinician who specializes in said disease and that is widely accepted by healthcare professionals. For example, standard of care therapies for NAFLD include methods of achieving weight loss, such as by altering a subject's diet, altering a subject's exercise routine, performing bariatric surgery on the subject, and/or administering a drug that induces weight loss in the subject.

As used herein, the terms “subject” and “patient” refer to an animal (e.g., a mammal, such as a human). A subject to be treated according to the methods described herein may be one who has been diagnosed with NAFLD or one at risk of developing this condition. Diagnosis may be performed by any method or technique known in the art or by a method described herein. One skilled in the art will understand that a subject to be treated according to the present disclosure may have been subjected to standard tests or may have been identified, without examination, as one at risk due to the presence of one or more risk factors associated with the disease or condition.

The term “synthetic” means produced, prepared, and/or manufactured by the hand of man. Synthesis of polynucleotides or polypeptides or other molecules of the present invention may be chemical or enzymatic.

As used herein, the terms “nonalcoholic fatty liver disease,” “NAFLD,” “steatotic liver disease,” “SLD,” “metabolic dysfunction-associated fatty liver disease,” “MAFLD,” “metabolic dysfunction-associated steatotic liver disease,” and “MASLD” refer to a condition in which fat (e.g., lipids) builds up in the liver due to causes other than excessive alcohol consumption. Nonalcoholic fatty liver (NAFL) and nonalcoholic steatohepatitis (NASH) are two types of NAFLD.

As used herein, the terms “agent” and “therapeutic agent” refer to an agent that can be used to treat a subject having NAFLD or at risk developing of NAFLD (e.g., at risk of NAFL, at risk of NASH, having NAFL, or having NASH). Therapeutic agents for use in the methods described herein are provided in Table 2 and Table 3 and are considered to be inhibitors (e.g., antibodies, small molecules, RNA molecules such as inhibitory RNA molecules or mRNA molecules encoding proteins, components of gene editing systems, and protein or peptide therapeutics) of a target listed in Table 1. The therapeutic agent, when administered to a subject, has a therapeutic and/or prophylactic effect and/or elicits a desired biological and/or pharmacological effect.

As used herein, “treatment” and “treating” refers to an approach for obtaining at least one of the following beneficial or desired results in a subject (e.g., a human subject): slowing or inhibiting the progression of NAFLD in a subject identified as having NAFLD (e.g., NAFL or NASH); reversing the progression of NAFLD in a subject identified as having NAFLD (e.g., NAFL or NASH); promoting the maintenance of NAFLD in a subject identified as having NAFLD (e.g., NAFL or NASH); slowing or inhibiting the onset of NASH in a subject identified as having NAFLD (e.g., NAFL); alleviating or ameliorating one or more symptoms (e.g., fatigue, weight loss, and/or jaundice) of NAFLD in a subject identified as having NAFLD (e.g., NAFL or NASH); reducing one or more clinical manifestations (e.g., liver steatosis, liver inflammation, and/or liver fibrosis) of NAFLD in a subject identified as having NAFLD (e.g., NAFL or NASH); slowing or inhibiting the onset of NAFL in a subject identified as at risk of developing NAFLD; alleviating or ameliorating one or more symptoms (e.g., fatigue, weight loss, and/or jaundice) of NAFLD in a subject identified as at risk of developing NAFLD (e.g., NAFL or NASH); slowing or inhibiting the progression of liver steatosis, liver inflammation, and/or liver fibrosis in a subject in need thereof (e.g., in a subject identified as having or at risk of developing NAFLD); reversing the progression of liver steatosis, liver inflammation, and/or liver fibrosis in a subject in need thereof (e.g., in a subject identified as having or at risk of developing NAFLD); and promoting the maintenance of liver steatosis, liver inflammation, and/or liver fibrosis in a subject in need thereof (e.g., in a subject identified as having or at risk of developing NAFLD). “Alleviating,” “ameliorating,” “reducing,” “reversing,” or “slowing” means that the extent of the disease, clinical manifestations of the disease, or symptoms of the disease are lessened such that a time course of disease progression is slowed, as compared to the absence of treatment. “Treatment” can also mean prolonging liver function and/or survival of the subject, as compared to not receiving treatment. The treatments described herein may reduce the number (e.g., mean number) and/or area (e.g., mean area) of lipid droplets present in one or more liver cells (e.g., hepatocytes (HCs)), reduce liver inflammation in the subject (e.g., the subject at risk of developing NAFLD or the subject having NAFLD), reduce liver fibrosis in the subject (e.g., the subject at risk of developing NAFLD or the subject having NAFLD), and/or reduce liver ballooning (e.g., in the subject at risk of developing NAFLD or the subject having NAFLD).

### BRIEF DESCRIPTION OF THE DRAWINGS

FIG. 1 is a representative graph of siRNA knockdown efficiency of a target gene. Knockdown of target complement C8 beta chain (C8B) with an siRNA leads to reduced expression of C8B compared to a non-targeting, scrambled siRNA (siNEG).

FIG. 2 is a representative graph of siRNA knockdown efficiency of a target gene. Knockdown of target solute carrier family 22 member 25 (SLC22A25) with an siRNA leads to reduced expression of SLC22A25 compared to a siNEG.

FIG. 3 is a representative plot of reduced lipid droplet accumulation after siRNA knockdown of C8B. Shown is the average lipid count per cell (left), the corresponding residuals after correcting for cell density (middle) and their marginal distributions (right). Each point represents a single field of view from a microscope. The solid line shows the expected lipid accumulation as a function of cell density, as estimated from siNEG images.

FIG. 4 is a representative plot of reduced lipid droplet accumulation after siRNA knockdown of SLC22A25. Shown is the average lipid count per cell (left), the corresponding residuals after correcting for cell density (middle) and their marginal distributions (right). Each point represents a single field of view from a microscope. The solid line shows the expected lipid accumulation as a function of cell density, as estimated from siNEG images.

### DETAILED DESCRIPTION

Described herein are methods of treating a subject that has been diagnosed with or at risk of developing non-alcoholic fatty liver disease (NAFLD), such as non-alcoholic fatty liver (NAFL) or non-alcoholic steatohepatitis (NASH). For example, the methods described herein may be used for treating a subject that is at risk of developing NAFLD. A subject may be at risk of developing NAFLD if they have a family history of NAFLD, are obese (have a body-mass index ≥30 kg/m²) or have a comorbid condition that affects lipid metabolism. Alternatively, the methods described herein may be used for treating a subject that has already developed some form of NAFLD, such as an NAFL or NASH; such methods can slow, inhibit, or even reverse further disease progression. Subjects may be diagnosed with NAFLD (e.g., NAFL or NASH) using standard clinical tests know in the art, such as: liver biopsy, magnetic resonance imaging (MRI) to detect or measure steatosis and/or hepatocellular ballooning, magnetic resonance elastography (MRE) to detect or measure fibrosis, an ultrasound to detect or measure steatosis and/or fibrosis, or other biomarker laboratory results).

Additionally, provided herein are compositions for use in the methods described herein. Specifically, the compositions described herein contain one or more therapeutic agents (e.g., an agent listed in Table 2, Table 3, or Table 4), which may include an inhibitory nucleic acid molecule, an RNA (e.g., an mRNA) encoding a protein that can inhibit or activate a target described herein, a soluble protein (e.g., a dominant negative protein or a soluble target binding partner that can modulate the activity of a target described herein), a small molecule, an antibody, or a component of a gene editing system.

### NAFLD

NAFLD, also called steatotic liver disease (SLD), metabolic dysfunction-associated fatty liver disease (MAFLD), or metabolic dysfunction-associated steatotic liver disease (MASLD), is a condition in which steatosis (e.g., hepatic steatosis) occurs in the liver. Steatosis, also called fatty change, is an abnormal retention of lipids (i.e., fat) within a cell (e.g., hepatocytes). When the liver, an organ responsible for lipid metabolism, has diagnosable features of steatosis (e.g., hepatic steatosis) that are not caused by a subject's excessive alcohol consumption, the subject is diagnosed as having NAFLD. Early stages of NAFLD, referred herein as NAFL, are hallmarked by hepatic steatosis (e.g., ≥5% hepatic steatosis), with little or no inflammation of the liver. Because there is little to no inflammation of the liver, the liver is not damaged, although it may be enlarged. Later stages of NAFLD, referred herein as NASH, are not only hallmarked by hepatic steatosis, but by also hepatocellular ballooning and inflammation of the liver, which can result in liver damage. Liver damage can lead to fibrosis, or scarring, of the tissue. If left unchecked, excessive scarring can lead to cirrhosis, which results in permanent damage to the liver and reduced liver function. Subjects that have NAFLD (e.g., subjects that have NAFLD with or without a diagnosis) may experience symptoms such as fatigue, weight loss, jaundice, fluid build-up (e.g., bloat and/or lymphedema), nausea, confusion, itching, or a combination thereof. Current methods of identifying subjects as having or at risk of having NAFLD include a biopsy of one or more tissues (e.g., from the liver), magnetic resonance imaging (MRI) to detect or measure steatosis, magnetic resonance elastography (MRE) to detect or measure fibrosis, an ultrasound to detect steatosis and/or fibrosis. Other NAFLD biomarkers and diagnostic modalities have been described elsewhere, such as, e.g., Martinou et al., Diagnostics 12(2):407, 2022, which is hereby incorporated by reference.

Risk factors for NAFLD or hepatic steatosis include other diseases or conditions. In some embodiments, a subject is at risk of developing NAFLD if they have a metabolic or hormonal disorder such as, e.g., type 2 diabetes or insulin resistance, hypothyroidism, hypopituitarism, polycystic ovarian syndrome, growth hormone deficiency, or dyslipidemia. In some embodiments, a subject may be at risk of developing NAFLD if they have low circulating levels of high-density lipoprotein (HDL)-cholesterol of about 500 mg/L or lower (e.g., about 500 mg/L, about 490 mg/L, about 480 mg/L, about 470 mg/L, about 460 mg/L, about 450 mg/L, about 440 mg/L, about 430 mg/L, about 420 mg/L, about 410 mg/L, about 400 mg/L, or lower than 400 mg/L). In some embodiments a subject may be at risk of developing NAFLD if they have high circulating levels of low-density lipoprotein (LDL)-cholesterol of about 1200 mg/L or higher (e.g., about 1200 mg/L, about 1250 mg/L, about 1300 mg/L, about 1350 mg/L, about 1400 mg/L, about 1450 mg/L, or higher than 1450 mg/L). In some embodiments, a subject at risk of developing NAFLD may have altered liver transaminase levels (e.g., elevated or altered AST/ALT ratios; e.g., an AST/ALT ratio of less than 1 or greater than or equal to 2). In some embodiments, a subject may be at risk of developing NAFLD if they have gout or high uric acid levels. In other embodiments, a subject may be at risk of developing NAFLD if they have gallstone disease or have had a cholecystectomy. In some embodiments, a subject may be at risk of developing NAFLD if they have a genetic predisposition such as mutations or polymorphisms in genes associated with NAFLD (e.g., PNPLA3, TM6SF2, SAMM-50, FDFT1, COL13A1, NCAN, GCKR, SREBF2, MBOAT7-TMC4, and/or HSD17B13). In further embodiments a subject may be at risk of developing NAFLD if they take certain medications such as amiodarone (CORDARONE®), diltiazem (CARDIZEM®), tamoxifen (NOLVADEX®), or steroids.

Other risk factors for NAFLD or liver steatosis are related to a subject's body type or lifestyle. In some embodiments, a subject may be at risk of developing NAFLD if they are above the age of 40 years old or have completed menopause. In some embodiments, a subject may be at risk of developing NAFLD if they have sarcopenia or skeletal muscle atrophy. In some embodiments, a subject may be at risk of developing NAFLD if they have poor diet, exercise, and sleep habits or if they have obstructive sleep apnea or sleep deprivation. In some embodiments, a subject may be at risk of developing NAFLD if they have high oxidative stress or a dysregulated gut microbiome. In some embodiments, a subject may be at risk of developing NAFLD if they have a BMI ≥30 kg/m² or a large waist circumference (e.g., about 80 cm or larger for males and about 78 cm or larger for females), wherein a large proportion of body fat is located in the abdomen. In some embodiments, a subject may be at risk of developing NAFLD if they have hypertension or blood pressure ≥130/85 mm Hg.

Standard treatment options for NAFLD include lifestyle changes such as, e.g., dietary changes to reduce cholesterol, regular exercise, and losing weight via modifications to diet and exercise routines, bariatric surgery, and/or medications to induce weight loss. Other standard care options include reducing liver inflammation or injury such as monitoring medications and reducing or eliminating alcohol intake. In instances where the disease is advanced (e.g., NASH progresses to cirrhosis or NASH severity increases), a subject may undergo a liver transplant.

### Targets for Treating NAFLD

The present disclosure is based, in part, on the discovery that the proteins listed in Table 1 modulate lipid accumulation (i.e., lipid droplet formation) in hepatocytes.

TABLE 1
Targets for treating NAFLD

| | | NCBI | mRNA/transcript | Protein | |
|---|---|---|---|---|---|
| Target Name | Gene Name | Gene ID | Sequence | Sequence | Splice Variants |
| Alpha kinase 2 | ALPK2 | 115701 | SEQ ID NO: 1 | SEQ ID NO: 2 | NM_052947.4 |
| Aminocarboxy | ACMSD | 130013 | SEQ ID NO: 3 | SEQ ID NO: 4 | NM_138326.3, |
| muconate | | | | | NM_001307983.2 |
| semialdehyde | | | | | |
| decarboxylase | | | | | |
| ATP binding | ABCB4 | 5244 | SEQ ID NO: 5 | SEQ ID: 6 | NM_000443.4, |
| cassette | | | | | NM_018849.3, |
| subfamily B | | | | | NM_018850.3 |
| member 4 | | | | | |
| Complement | CFB | 629 | SEQ ID NO: 7 | SEQ ID NO: 8 | NM_001710.6 |
| factor B | | | | SEQ ID NO: 9 | |
| Complement | C8B | 732 | SEQ ID NO: 510 | SEQ ID NO: 11 | NM_001278543.2, |
| C8 beta chain | | | | SEQ ID NO: 12 | NM_001278544.2, |
| | | | | | NM_000066.4 |
| Contactin 3 | CNTN3 | 5067 | SEQ ID NO: 13 | SEQ ID NO: 14 | NM_020872.3, |
| | | | | SEQ ID NO: 15 | NM_001393376.1 |
| Haptoglobin- | HPR | 3250 | SEQ ID NO: 16 | SEQ ID NO: 17 | NM_020995.4, |
| related protein | | | | | NM_001384360.1 |
| Homogentisate | HGD | 3081 | SEQ ID NO: 18 | SEQ ID NO: 19 | NM_000187.4 |
| 1,2- | | | | | |
| dioxygenase | | | | | |
| Methylmalonyl- | MCEE | 84693 | SEQ ID NO: 20 | SEQ ID NO: 21 | NM_032601.4 |
| CoA epimerase | | | | SEQ ID NO: 22 | |
| Perilipin 5 | PLIN5 | 440503 | SEQ ID NO: 23 | SEQ ID NO: 24 | NM_001013706.3 |
| Peroxisomal | PEX6 | 5190 | SEQ ID NO: 25 | SEQ ID NO: 26 | NM_000287.4, |
| biogenesis | | | | | NM_001316313.2, |
| factor 6 | | | | | NR_133009.2 |
| PRELI domain | PRELID2 | 153768 | SEQ ID NO: 27 | SEQ ID NO: 28 | NM_205846.3, |
| containing 2 | | | | | NM_138492.6, |
| | | | | | NM_182960.4 |
| RAB11A, | RAB11A | 8766 | SEQ ID NO: 29 | SEQ ID NO: 30 | NM_004663.5, |
| Member RAS | | | | | NM_001206836.2 |
| Oncogene | | | | | |
| Family | | | | | |
| RERG like | RERGL | 79785 | SEQ ID NO: 31 | SEQ ID NO: 32 | NM_001286201.2, |
| protein | | | | | NM_024730.4, |
| | | | | | NR_104413.1 |
| SH3 and PX | SH3PXD2A | 9644 | SEQ ID NO: 33 | SEQ ID NO: 34 | NM_001394015.1, |
| domains 2A | | | | | NM_014631.3, |
| | | | | | NM_001365079.1, |
| | | | | | NM_001394016.1, |
| | | | | | NM_001394017.1, |
| | | | | | NM_001394018.1, |
| | | | | | NM_001394019.1, |
| | | | | | NM_001394020.1, |
| | | | | | NM_001394021.1, |
| | | | | | NM_001394022.1, |
| | | | | | NM_001394023.1 |
| Solute carrier | SLC16A10 | 117247 | SEQ ID NO: 35 | SEQ ID NO: 36 | NM_018593.5 |
| family 16 | | | | | |
| member 10 | | | | | |
| Solute carrier | SLC22A25 | 387601 | SEQ ID NO: 37 | SEQ ID NO: 38 | NM_199352.6, |
| family 22 | | | | | NM_001394059.1, |
| member 25 | | | | | NM_001394058.1 |
| Transthyretin | TTR | 7276 | SEQ ID NO: 39 | SEQ ID NO: 40 | NM_000371.4 |
| Tumor protein | TPRG1 | 285386 | SEQ ID NO: 41 | SEQ ID NO: 42 | NM_198485.4 |
| p63 regulated 1 | | | | | |

### Inhibitory Agents

Agents that inhibit the target listed in Table 1 can be used to treat a subject having or at risk of developing NAFLD (e.g., NAFL or NASH). The agents described herein (e.g., see Table 2, Table 3, or Table 4) may provide a therapeutic effect by inhibiting a target listed in Table 1. A list of therapeutic platforms and agents for inhibiting targets listed in Table 1 is provided in Table 2 below.

TABLE 2
Therapeutic Platforms and Agents for Treating NAFLD

| Platform | Agent |
|---|---|
| Inhibitory Nucleic Acid | siRNA |
| Molecule | dsRNA |
| | miRNA |
| | shRNA |
| | ASO |
| | GapmeR |
| Antagonist | mRNA-encoded protein |
| | Soluble protein/antibody |
| | Small molecule |
| Programmable Nuclease | CRISPR-associated (CAS) protein |
| | Transcription activator-like effector nuclease |
| | (TALEN) |
| | Zinc finger nuclease |

Exemplary inhibitors (e.g., see Table 3 and Table 4) include inhibitory nucleic acid molecules (e.g., siRNAs, dsRNAs, miRNAs, shRNAs, ASOs, and gapmers), RNA molecules that encode inhibitory proteins, soluble proteins that inhibit the activity of a target or the binding of a target to a binding partner, small molecule inhibitors, inhibitory antibodies, and programmable nucleases for editing and inhibiting gene expression (e.g., CRISPR-Cas mediated gene inhibition). Each inhibitor is described in further detail below.

TABLE 3
Exemplary Targeting Agents

| | Agent | | | | |
|---|---|---|---|---|---|
| | | | Protein/Peptide | | |
| Target | ASO | siRNA | Inhibitor | Antibody | Small molecules |
| ABCB4 | | | | | Cyclosporine A |
| | | | | | Valspodar |
| | | | | | Verapamil |
| | | | | | Vinblastine |
| | | | | | Paclitaxel |
| | | | | | Itraconazole |
| CFB | IONIS-FB-LRx | | | SAR-443809 | Iptacopan (LNP023) |
| | | | | NM-8074 | |
| ACMSD | | | | | TES-991 |
| RAB11A | | | RFP26 | | |
| TTR | | Inotersen | | | |
| | | Patisiran | | | |

### Inhibitory Nucleic Acid Molecules

In some embodiments, the therapeutic agent is an inhibitory nucleic acid molecule that reduces the expression or activity of one or more of the targets shown in Table 1. In some embodiments, the therapeutic agent is an inhibitory nucleic acid molecule that reduces the expression or activity of one or more of the following targets: ATP binding cassette subfamily B member 4 (ABCB4), complement C8 beta chain (C8B), homogentisate 1,2-dioxygenase (HGD), methylmalonyl-CoA epimerase (MCEE), SH3 and PX domains 2A (SH3PXD2A), solute carrier family 16 member 10 (SLC16A10), transthyretin (TTR), haptoglobin-related protein (HPR), peroxisomal biogenesis factor 6 (PEX6), RAB11A, Member RAS Oncogene Family (RAB11A), and solute carrier family 22 member 25 (SLC22A25).

In some embodiments, the therapeutic agent is an inhibitory nucleic acid molecule that reduces the expression or activity of ABCB4. In some embodiments, the therapeutic agent is an inhibitory nucleic acid molecule that reduces the expression or activity of C8B. In some embodiments, the therapeutic agent is an inhibitory nucleic acid molecule that reduces the expression or activity of HGD. In some embodiments, the therapeutic agent is an inhibitory nucleic acid molecule that reduces the expression or activity of MCEE. In some embodiments, the therapeutic agent is an inhibitory nucleic acid molecule that reduces the expression or activity of SH3PXD2A. In some embodiments, the therapeutic agent is an inhibitory nucleic acid molecule that reduces the expression or activity of SLC16A10. In some embodiments, the therapeutic agent is an inhibitory nucleic acid molecule that reduces the expression or activity of TTR. In some embodiments, the therapeutic agent is an inhibitory nucleic acid molecule that reduces the expression or activity of HPR. In some embodiments, the therapeutic agent is an inhibitory nucleic acid molecule that reduces the expression or activity of PEX6. In some embodiments, the therapeutic agent is an inhibitory nucleic acid molecule that reduces the expression or activity of RAB11A. In some embodiments, the therapeutic agent is an inhibitory nucleic acid molecule that reduces the expression or activity of SLC22A25.

Exemplary inhibitory nucleic acid molecules are siRNAs, dsRNAs, miRNAs, shRNAs, ASOs, and gapmers; however, any nucleic acid molecule capable of reducing mRNA and/or protein expression of a target is envisioned for use in the methods described herein. The inhibitory nucleic acid molecules of the disclosure may be referred to as RNA inhibitory (RNAi) molecules (e.g., siRNA, dsRNA, miRNA, and shRNA).

In some embodiments, the inhibitory RNA has a nucleobase sequence containing a portion of at least 8 contiguous nucleobases (e.g., 8, 9, 10, 11, 12, 13, 14, 15, 16, 17, 18, 19, 20, 21, 22, 23, 24, 25, 26, 27, 28, 29, or 30 nucleobases) having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to an equal length portion of a target region of an mRNA transcript of a human gene encoding a target to be inhibited (e.g., an mRNA transcript referenced in Table 1 or a variant thereof. In some embodiments, the inhibitory RNA has a nucleobase sequence containing a portion of at least 8 contiguous nucleobases (e.g., 8, 9, 10, 11, 12, 13, 14, 15, 16, 17, 18, 19, 20, 21, 22, 23, 24, 25, 26, 27, 28, 29, or 30 nucleobases) having 100% complementarity to an equal length portion of a target region of an mRNA transcript (including splice variants) referenced in Table 1 (e.g., an mRNA transcript encoding ABCB4, C8B, HGD, MCEE, SH3PXD2A, SLC16A10, TTR, HPR, PEX6, RAB11A, or SLC22A25) or a variant thereof (e.g., an mRNA transcript having at least 70%, 75%, 80%, 85%, 86%, 87%, 88%, 89%, 90%, 91%, 92%, 93%, 94%, 95%, 96%, 97%, 98%, or 99% sequence identity to ABCB4, C8B, HGD, MCEE, SH3PXD2A, SLC16A10, TTR, HPR, PEX6, RAB11A, or SLC22A25).

In some embodiments, the inhibitory nucleic acid molecule comprises a sequence complementary to at least 8 contiguous nucleotides set forth within at least one of the mRNA transcripts referenced in Table 1 or a variant thereof. In some embodiments, the inhibitory nucleic acid molecule comprises a sequence complementary to at least 9 contiguous nucleotides set forth within at least one of the mRNA transcripts referenced in Table 1 or a variant thereof. In some embodiments, the inhibitory nucleic acid molecule comprises a sequence complementary to at least 10 contiguous nucleotides set forth within at least one of the mRNA transcripts referenced in Table 1 or a variant thereof. In some embodiments, the inhibitory nucleic acid molecule comprises a sequence complementary to at least 11 contiguous nucleotides set forth within at least one of the mRNA transcripts referenced in Table 1 or a variant thereof. In some embodiments, the inhibitory nucleic acid molecule comprises a sequence complementary to at least 12 contiguous nucleotides set forth within at least one of the mRNA transcripts referenced in Table 1 or a variant thereof. In some embodiments, the inhibitory nucleic acid molecule comprises a sequence complementary to at least 13 contiguous nucleotides set forth within at least one of the mRNA transcripts referenced in Table 1 or a variant thereof. In some embodiments, the inhibitory nucleic acid molecule comprises a sequence complementary to at least 14 contiguous nucleotides set forth within at least one of the mRNA transcripts referenced in Table 1 or a variant thereof. In some embodiments, the inhibitory nucleic acid molecule comprises a sequence complementary to at least 15 contiguous nucleotides set forth within at least one of the mRNA transcripts referenced in Table 1 or a variant thereof. In some embodiments, the inhibitory nucleic acid molecule comprises a sequence complementary to at least 16 contiguous nucleotides set forth within at least one of the mRNA transcripts referenced in Table 1 or a variant thereof. In some embodiments, the inhibitory nucleic acid molecule comprises a sequence complementary to at least 17 contiguous nucleotides set forth within at least one of the mRNA transcripts referenced in Table 1 or a variant thereof. In some embodiments, the inhibitory nucleic acid molecule comprises a sequence complementary to at least 18 contiguous nucleotides set forth within at least one of the mRNA transcripts referenced in Table 1 or a variant thereof. In some embodiments, the inhibitory nucleic acid molecule comprises a sequence complementary to at least 19 contiguous nucleotides set forth within at least one of the mRNA transcripts referenced in Table 1 or a variant thereof. In some embodiments, the inhibitory nucleic acid molecule comprises a sequence complementary to at least 20 contiguous nucleotides set forth within at least one of the mRNA transcripts referenced in Table 1 or a variant thereof. In some embodiments, the inhibitory nucleic acid molecule comprises a sequence complementary to at least 21 contiguous nucleotides set forth within at least one of the mRNA transcripts referenced in Table 1 or a variant thereof. In some embodiments, the inhibitory nucleic acid molecule comprises a sequence complementary to at least 22 contiguous nucleotides set forth within at least one of the mRNA transcripts referenced in Table 1 or a variant thereof. In some embodiments, the inhibitory nucleic acid molecule comprises a sequence complementary to at least 23 contiguous nucleotides set forth within at least one of the mRNA transcripts referenced in Table 1 or a variant thereof. In some embodiments, the inhibitory nucleic acid molecule comprises a sequence complementary to at least 24 contiguous nucleotides set forth within at least one of the mRNA transcripts referenced in Table 1 or a variant thereof. In some embodiments, the inhibitory nucleic acid molecule comprises a sequence complementary to at least 25 contiguous nucleotides set forth within at least one of the mRNA transcripts referenced in Table 1 or a variant thereof. In some embodiments, the inhibitory nucleic acid molecule comprises a sequence complementary to at least 26 contiguous nucleotides set forth within at least one of the mRNA transcripts referenced in Table 1 or a variant thereof. In some embodiments, the inhibitory nucleic acid molecule comprises a sequence complementary to at least 27 contiguous nucleotides set forth within at least one of the mRNA transcripts referenced in Table 1 or a variant thereof. In some embodiments, the inhibitory nucleic acid molecule comprises a sequence complementary to at least 28 contiguous nucleotides set forth within at least one of the mRNA transcripts referenced in Table 1 or a variant thereof. In some embodiments, the inhibitory nucleic acid molecule comprises a sequence complementary to at least 29 contiguous nucleotides set forth within at least one of the mRNA transcripts referenced in Table 1 or a variant thereof. In some embodiments, the inhibitory nucleic acid molecule comprises a sequence complementary to at least 30 contiguous nucleotides set forth within at least one of the mRNA transcripts referenced in Table 1 or a variant thereof. In some embodiments, the inhibitory nucleic acid molecule comprises a sequence complementary to at least 31 contiguous nucleotides set forth within at least one of the mRNA transcripts referenced in Table 1 or a variant thereof. In some embodiments, the inhibitory nucleic acid molecule comprises a sequence complementary to at least 32 contiguous nucleotides set forth within at least one of the mRNA transcripts referenced in Table 1 or a variant thereof. In some embodiments, the inhibitory nucleic acid molecule comprises a sequence complementary to at least 33 contiguous nucleotides set forth within at least one of the mRNA transcripts referenced in Table 1 or a variant thereof. In some embodiments, the inhibitory nucleic acid molecule comprises a sequence complementary to at least 34 contiguous nucleotides set forth within at least one of the mRNA transcripts referenced in Table 1 or a variant thereof. In some embodiments, the inhibitory nucleic acid molecule comprises a sequence complementary to at least 35 contiguous nucleotides set forth within at least one of the mRNA transcripts referenced in Table 1 or a variant thereof. In some embodiments, the inhibitory nucleic acid molecule comprises a sequence complementary to at least 36 contiguous nucleotides set forth within at least one of the mRNA transcripts referenced in Table 1 or a variant thereof. In some embodiments, the inhibitory nucleic acid molecule comprises a sequence complementary to at least 37 contiguous nucleotides set forth within at least one of the mRNA transcripts referenced in Table 1 or a variant thereof. In some embodiments, the inhibitory nucleic acid molecule comprises a sequence complementary to at least 38 contiguous nucleotides set forth within at least one of the mRNA transcripts referenced in Table 1 or a variant thereof. In some embodiments, the inhibitory nucleic acid molecule comprises a sequence complementary to at least 39 contiguous nucleotides set forth within at least one of the mRNA transcripts referenced in Table 1 or a variant thereof. In some embodiments, the inhibitory nucleic acid molecule comprises a sequence complementary to at least 40 contiguous nucleotides set forth within at least one of the mRNA transcripts referenced in Table 1 or a variant thereof. In some embodiments, the inhibitory nucleic acid molecule comprises a sequence complementary to at least 41 contiguous nucleotides set forth within at least one of the mRNA transcripts referenced in Table 1 or a variant thereof. In some embodiments, the inhibitory nucleic acid molecule comprises a sequence complementary to at least 42 contiguous nucleotides set forth within at least one of the mRNA transcripts referenced in Table 1 or a variant thereof. In some embodiments, the inhibitory nucleic acid molecule comprises a sequence complementary to at least 43 contiguous nucleotides set forth within at least one of the mRNA transcripts referenced in Table 1 or a variant thereof. In some embodiments, the inhibitory nucleic acid molecule comprises a sequence complementary to at least 44 contiguous nucleotides set forth within at least one of the mRNA transcripts referenced in Table 1 or a variant thereof. In some embodiments, the inhibitory nucleic acid molecule comprises a sequence complementary to at least 45 contiguous nucleotides set forth within at least one of the mRNA transcripts referenced in Table 1 or a variant thereof. In some embodiments, the inhibitory nucleic acid molecule comprises a sequence complementary to at least 46 contiguous nucleotides set forth within at least one of the mRNA transcripts referenced in Table 1 or a variant thereof. In some embodiments, the inhibitory nucleic acid molecule comprises a sequence complementary to at least 47 contiguous nucleotides set forth within at least one of the mRNA transcripts referenced in Table 1 or a variant thereof. In some embodiments, the inhibitory nucleic acid molecule comprises a sequence complementary to at least 48 contiguous nucleotides set forth within at least one of the mRNA transcripts referenced in Table 1 or a variant thereof. In some embodiments, the inhibitory nucleic acid molecule comprises a sequence complementary to at least 49 contiguous nucleotides set forth within at least one of the mRNA transcripts referenced in Table 1 or a variant thereof. In some embodiments, the inhibitory nucleic acid molecule comprises a sequence complementary to at least 50 contiguous nucleotides set forth within at least one of the mRNA transcripts referenced in Table 1 or a variant thereof. A variant thereof includes an mRNA transcript having at least 70%, 75%, 80%, 85%, 86%, 87%, 88%, 89%, 90%, 91%, 92%, 93%, 94%, 95%, 96%, 97%, 98%, or 99% sequence identity to an mRNA transcript (including splice variant mRNA transcripts) referenced in Table 1 (e.g., an mRNA encoding ABCB4, C8B, HGD, MCEE, SH3PXD2A, SLC16A10, TTR, HPR, PEX6, RAB11A, or SLC22A25).

In some embodiments, the inhibitory nucleic acid is an siRNA inhibiting a target of Table 1. In some embodiments, the inhibitory nucleic acid is an dsRNA inhibiting a target of Table 1. In some embodiments, the inhibitory nucleic acid is an ASO inhibiting a target of Table 1. In some embodiments, the inhibitory nucleic acid is a miRNA inhibiting a target of Table 1. In some embodiments, the inhibitory nucleic acid is an shRNA inhibiting a target of Table 1. In some embodiments, the inhibitory nucleic acid is a gapmer inhibiting a target of Table 1. Each of these inhibitory nucleic acid molecules is described in further detail below. In some embodiments, the siRNA, dsRNA, ASO, miRNA, shRNA, or gapmer inhibits the expression of ABCB4, C8B, HGD, MCEE, SH3PXD2A, SLC16A10, TTR, HPR, PEX6, RAB11A, or SLC22A25.

### Small Interfering RNAs

An siRNA molecule of the disclosure is a single-stranded (ss) or double-stranded (ds) nucleic acid molecule made of DNA, RNA, or both DNA and RNA (e.g., a chimeric) nucleosides that are complementary to an mRNA transcript (e.g., a transcript referenced in Table 1) encoding a target, or a portion or variant thereof, and prevents translation of the mRNA into a protein. Once an siRNA molecule enters a cell, it is incorporated into an RNA-induced silencing complex (RISC). Upon hybridization of an antisense strand of the siRNA molecule to a target mRNA (e.g., an mRNA transcript referenced in Table 1), the RISC complex will cleave the target mRNA, thereby inactivating the target mRNA, resulting in reduced mRNA and protein levels of the target. In some instances, an siRNA inhibits translation of the mRNA into a protein via translation repression or deadenylation-dependent decay mechanisms.

In some embodiments, an siRNA molecule of the disclosure may include a nucleotide sequence of about 10 to about 30 nucleotides in length (e.g., 9, about 10, about 11, about 12, about 13, about 14, about 15, about 16, about 17, about 18, about 19, about 20, about 21, about 22, about 23, about 24, about 25, about 26, about 27, about 28, about 29, about 30, or 31 nucleotides in length).

In some embodiments, an siRNA molecule of the disclosure may include a nucleotide sequence of 10 to 30 nucleotides in length (e.g., 10, 11, 12, 13, 14, 15, 16, 17, 18, 19, 20, 21, 22, 23, 24, 25, 26, 27, 28, 29, or 30 nucleotides in length).

It is within the scope of the disclosure that any length, known and previously unknown in the art, may be employed for the current invention. Exemplary siRNA molecules are inotersen and patisiran, each of which target TTR. Additional exemplary siRNA molecules are the siRNA molecules listed in Table 4.

In some embodiments, an siRNA molecule of the disclosure contains a sequence complementary to at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides set forth within at least one of the mRNA transcripts referenced in Table 1 or a variant thereof.

In some embodiments, an siRNA molecule of the disclosure contains an antisense strand. In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, an siRNA molecule of the disclosure contains a sense strand. In some embodiments, the sense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

In some embodiments, the sense and antisense strands of an siRNA molecule of the disclosure are completely complementary to one another. In some embodiments, the sense and antisense strands of an siRNA molecule of the disclosure are completely complementary to the extent that their lengths overlap with one another. Depending on the sequence of the sense and antisense strand, complementarity need not be complete or perfect, which means that the sense and antisense strand are not 100% base-paired due to mismatches. One or more (e.g., 1, 2, 3, 4, or 5) mismatches may be present within the ds siRNA molecule without impacting the siRNA molecule's ability to reduced expression of a target's mRNA transcript and/or protein.

The nucleotide sequence of an siRNA molecule of the disclosure may contain sufficient complementarity to a portion of a transcript (e.g., a transcript referenced in Table 1 or variant thereof) encoding a target such that the siRNA molecule can hybridize with one or more transcripts encoding the target. In some embodiments, the siRNA molecule is at least 70%, at least 75%, at least 80%, at least 85%, at least 90%, at least 95%, at least 98%, or at least 99% complementary to one or more transcripts encoding a target (e.g., a transcript referenced in Table 1 or variants thereof), or a portion thereof. In some embodiments, the siRNA molecule is 100% complementary to one or more transcripts encoding a target (e.g., a transcript referenced in Table 1 or variants thereof), or a portion thereof.

In some embodiments, the nucleotide sequence of an siRNA molecule of the disclosure may contain sufficient complementarity to an exon sequence within one or more transcripts (e.g., a transcript listed in Table 1 encoding a target, or a portion thereof. In some embodiments, the nucleotide sequence of an siRNA molecule may contain sufficient complementarity to an intron sequence within one or more transcripts encoding a target, or a portion thereof. In some embodiments, an siRNA molecule of the disclosure may contain sufficient complementarity to a pre-mRNA transcript or an mRNA transcript encoding any one of the targets listed in Table 1 (e.g., a transcript referenced in Table 1 or a variant thereof).

In some embodiments, the nucleotide sequence of an siRNA molecule of the disclosure may contain a sense strand and/or and antisense strand having at least 70%, at least 75%, at least 80%, at least 85%, at least 90%, at least 95%, at least 98%, or at least 99% sequence identity to any one or more of SEQ ID NOs: 43-194 (e.g., see Table 4).

In some embodiments, an siRNA molecule described herein may have 0-7 nucleotide 3′ overhangs or 0-4 nucleotide 5′ overhangs. In some embodiments, the siRNA molecule has a single uracil overhang at one or more 3′ ends of the siRNA. In some embodiments, the siRNA molecule has a double uracil overhang at one or more 3′ ends of the siRNA. In some embodiments, the siRNA molecule has a single thymine overhang at one or more 3′ ends of the siRNA. In some embodiments, the siRNA molecule has a double thymine overhang at one or more 3′ ends of the siRNA. In some embodiments, the siRNA molecule has a single cytosine and a single thymine (e.g., CT) overhang at one or more 3′ ends of the siRNA.

For any of the methods described herein, different siRNA molecules of the disclosure can be combined for decreasing one or more transcripts (e.g., a transcript listed in Table 1) of one or more targets described herein (e.g., one or more targets listed in Table 1). A combination oftwo or more siRNA molecules may be used in a method of the invention, such as two different siRNA molecules, three different siRNA molecules, four different siRNA molecules, five different siRNA molecules, or more, to inhibit the same target transcript (e.g., a transcript referenced in Table 1 or variant thereof). Alternatively, two different siRNA molecules, three different siRNA molecules, four different siRNA molecules, five different siRNA molecules, or more, to inhibit different target transcripts (e.g., two or more transcripts listed in Table 1 or variants thereof) may be used in a method of the invention.

TABLE 4
Exemplary siRNA Sequences

| Name | Strand Name | SEQ ID NO: | Sequence (5′ to 3′) |
|---|---|---|---|
| siRNA 1 | ALPK2-Antisense 1 | SEQ ID NO: 43 | tactgattcatccatgttg |
| | ALPK2-Sense 1 | SEQ ID NO: 44 | caacatggatgaatcagta |
| siRNA 2 | ALPK2-Antisense 2 | SEQ ID NO: 45 | taaatgccaaggtcttcgc |
| | ALPK2-Sense 2 | SEQ ID NO: 46 | gcgaagaccttggcattta |
| siRNA 3 | ALPK2-Antisense 3 | SEQ ID NO: 47 | ttatctctgggcaaagttc |
| | ALPK2-Sense 3 | SEQ ID NO: 48 | gaactttgcccagagataa |
| siRNA 4 | ALPK2-Antisense 4 | SEQ ID NO: 49 | tagcagttaatgttgttgg |
| | ALPK2-Sense 4 | SEQ ID NO: 50 | ccaacaacattaactgcta |
| siRNA 5 | ACMSD-Antisense 1 | SEQ ID NO: 51 | tctcgcaccactctgaaga |
| | ACMSD-Sense 1 | SEQ ID NO: 52 | tcttcagagtggtgcgaga |
| siRNA 6 | ACMSD-Antisense 2 | SEQ ID NO: 53 | taatacgaacttctggatc |
| | ACMSD-Sense 2 | SEQ ID NO: 54 | gatccagaagttcgtatta |
| siRNA 7 | ACMSD-Antisense 3 | SEQ ID NO: 55 | cattcgtccatccatctgc |
| | ACMSD-Sense 3 | SEQ ID NO: 56 | gcagatggatggacgaatg |
| siRNA 8 | ACMSD-Antisense 4 | SEQ ID NO: 57 | aaccaaggtatttcttcgg |
| | ACMSD-Sense 4 | SEQ ID NO: 58 | ccgaagaaataccttggtt |
| siRNA 9 | ABCB4-Antisense 1 | SEQ ID NO: 59 | tgatccgtgagctatggcc |
| | ABCB4-Sense 1 | SEQ ID NO: 60 | ggccatagctcacggatca |
| siRNA 10 | ABCB4-Antisense 2 | SEQ ID NO: 61 | taagcagctagttctttgt |
| | ABCB4-Sense 2 | SEQ ID NO: 62 | acaaagaactagctgctta |
| siRNA 11 | ABCB4-Antisense 3 | SEQ ID NO: 63 | tagcaatagggttaactgc |
| | ABCB4-Sense 3 | SEQ ID NO: 64 | gcagttaaccctattgcta |
| siRNA 12 | ABCB4-Antisense 4 | SEQ ID NO: 65 | aaagtgaacatcattgaac |
| | ABCB4-Sense 4 | SEQ ID NO: 66 | gttcaatgatgttcacttt |
| siRNA 13 | CFB-Antisense 1 | SEQ ID NO: 67 | taggactacttctatctcc |
| | CFB-Sense 1 | SEQ ID NO: 68 | ggagatagaagtagtccta |
| siRNA 14 | CFB-Antisense 2 | SEQ ID NO: 69 | ttatagaaaacccaaatcc |
| | CFB-Sense 2 | SEQ ID NO: 70 | ggatttgggttttctataa |
| siRNA 15 | CFB-Antisense 3 | SEQ ID NO: 71 | ttgaatgaaacgacttctc |
| | CFB-Sense 3 | SEQ ID NO: 72 | gagaagtcgtttcattcaa |
| siRNA 16 | CFB-Antisense 4 | SEQ ID NO: 73 | tagacatccagataatcct |
| | CFB-Sense 4 | SEQ ID NO: 74 | aggattatctggatgtcta |
| siRNA 17 | C8B-Antisense 1 | SEQ ID NO: 75 | tacctctgcatttgcctac |
| | C8B-Sense 1 | SEQ ID NO: 76 | gtaggcaaatgcagaggta |
| siRNA 18 | C8B-Antisense 2 | SEQ ID NO: 77 | caagtaggcatacctgtac |
| | C8B-Sense 2 | SEQ ID NO: 78 | gtacaggtatgcctacttg |
| siRNA 19 | C8B-Antisense 3 | SEQ ID NO: 79 | gattgttacactgcctttg |
| | C8B-Sense 3 | SEQ ID NO: 80 | caaaggcagtgtaacaatc |
| siRNA 20 | C8B-Antisense 4 | SEQ ID NO: 81 | tcctaatatagtgtttgcc |
| | C8B-Sense 4 | SEQ ID NO: 82 | ggcaaacactatattagga |
| siRNA 21 | CNTN3-Antisense 1 | SEQ ID NO: 83 | taaatcaccagatgaactc |
| | CNTN3-Sense 1 | SEQ ID NO: 84 | gagttcatctggtgattta |
| siRNA 22 | CNTN3-Antisense 2 | SEQ ID NO: 85 | caaatctccgactatcttc |
| | CNTN3-Sense 2 | SEQ ID NO: 86 | gaagatagtcggagatttg |
| siRNA 23 | CNTN3-Antisense 3 | SEQ ID NO: 87 | taatttcccacatcagacg |
| | CNTN3-Sense 3 | SEQ ID NO: 88 | cgtctgatgtgggaaatta |
| siRNA 24 | CNTN3-Antisense 4 | SEQ ID NO: 89 | agaagcaacaactttgagc |
| | CNTN3-Sense 4 | SEQ ID NO: 90 | gctcaaagttgttgcttct |
| siRNA 25 | HPR-Antisense 1 | SEQ ID NO: 91 | atatccgtgacatcattgc |
| | HPR-Sense 1 | SEQ ID NO: 92 | gcaatgatgtcacggatat |
| siRNA 26 | HPR-Antisense 2 | SEQ ID NO: 93 | taatgcgttatgcaatcgt |
| | HPR-Sense 2 | SEQ ID NO: 94 | acgattgcataacgcatta |
| siRNA 27 | HPR-Antisense 3 | SEQ ID NO: 95 | ttaaggtgtatactccatc |
| | HPR-Sense 3 | SEQ ID NO: 96 | gatggagtatacaccttaa |
| siRNA 28 | HPR-Antisense 4 | SEQ ID NO: 97 | gctccacatagccatttgc |
| | HPR-Sense 4 | SEQ ID NO: 98 | gcaaatggctatgtggagc |
| siRNA 29 | HGD-Antisense 1 | SEQ ID NO: 99 | tcttattggtgctccgtgg |
| | HGD-Sense 1 | SEQ ID NO: 100 | ccacggagcaccaataaga |
| siRNA 30 | HGD-Antisense 2 | SEQ ID NO: 101 | tattaatgaccgtgtaacc |
| | HGD-Sense 2 | SEQ ID NO: 102 | ggttacacggtcattaata |
| siRNA 31 | HGD-Antisense 3 | SEQ ID NO: 103 | tgaaggtagaatcctatac |
| | HGD-Sense 3 | SEQ ID NO: 104 | gtataggattctaccttca |
| siRNA 32 | HGD-Antisense 4 | SEQ ID NO: 105 | tctgaagaacactcattcc |
| | HGD-Sense 4 | SEQ ID NO: 106 | ggaatgagtgttcttcaga |
| siRNA 33 | MCEE-Antisense 1 | SEQ ID NO: 107 | aagccctacggcattcgcg |
| | MCEE-Sense 1 | SEQ ID NO: 108 | cgcgaatgccgtagggctt |
| siRNA 34 | MCEE-Antisense 2 | SEQ ID NO: 109 | ttcacttagactgcggatc |
| | MCEE-Sense 2 | SEQ ID NO: 110 | gatccgcagtctaagtgaa |
| siRNA 35 | MCEE-Antisense 3 | SEQ ID NO: 111 | attaatattatccacctcg |
| | MCEE-Sense 3 | SEQ ID NO: 112 | cgaggtggataatattaat |
| siRNA 36 | MCEE-Antisense 4 | SEQ ID NO: 113 | agaacctgtcacttgatcc |
| | MCEE-Sense 4 | SEQ ID NO: 114 | ggatcaagtgacaggttct |
| siRNA 37 | PLIN5-Antisense 1 | SEQ ID NO: 115 | actgtaaacatcgcagacc |
| | PLIN5-Sense 1 | SEQ ID NO: 116 | ggtctgcgatgtttacagt |
| siRNA 38 | PLIN5-Antisense 2 | SEQ ID NO: 117 | atctgagccgcctcttctt |
| | PLIN5-Sense 2 | SEQ ID NO: 118 | aagaagaggcggctcagat |
| siRNA 39 | PLIN5-Antisense 3 | SEQ ID NO: 119 | tctgcgtcagcctcacaac |
| | PLIN5-Sense 3 | SEQ ID NO: 120 | gttgtgaggctgacgcaga |
| siRNA 40 | PLIN5-Antisense 4 | SEQ ID NO: 121 | tcttcgggaggctgttact |
| | PLIN5-Sense 4 | SEQ ID NO: 122 | agtaacagcctcccgaaga |
| siRNA 41 | PEX6-Antisense 1 | SEQ ID NO: 123 | aatttcgatgtgtaactct |
| | PEX6-Sense 1 | SEQ ID NO: 124 | agagttacacatcgaaatt |
| siRNA 42 | PEX6-Antisense 2 | SEQ ID NO: 125 | tacatgttaatgagctctg |
| | PEX6-Sense 2 | SEQ ID NO: 126 | cagagctcattaacatgta |
| siRNA 43 | PEX6-Antisense 3 | SEQ ID NO: 127 | tctacttgcccaattgttg |
| | PEX6-Sense 3 | SEQ ID NO: 128 | caacaattgggcaagtaga |
| siRNA 44 | PEX6-Antisense 4 | SEQ ID NO: 129 | cagtctatcagagaggtcc |
| | PEX6-Sense 4 | SEQ ID NO: 130 | ggacctctctgatagactg |
| siRNA 45 | PRELID2-Antisense 1 | SEQ ID NO: 131 | tctttaatcatcataacgc |
| | PRELID2-Sense 1 | SEQ ID NO: 132 | gcgttatgatgattaaaga |
| siRNA 46 | PRELID2-Antisense 2 | SEQ ID NO: 133 | actccgtatggccatgttt |
| | PRELID2-Sense 2 | SEQ ID NO: 134 | aaacatggccatacggagt |
| siRNA 47 | PRELID2-Antisense 3 | SEQ ID NO: 135 | atgaactctgtccaatttg |
| | PRELID2-Sense 3 | SEQ ID NO: 136 | caaattggacagagttcat |
| siRNA 48 | PRELID2-Antisense 4 | SEQ ID NO: 137 | tacggcttagcttcaacat |
| | PRELID2-Sense 4 | SEQ ID NO: 138 | atgttgaagctaagccgta |
| siRNA 49 | RAB11A-Antisense 1 | SEQ ID NO: 139 | ataggaaccacattgttgc |
| | RAB11A-Sense 1 | SEQ ID NO: 140 | gcaacaatgtggttcctat |
| siRNA 50 | RAB11A-Antisense 2 | SEQ ID NO: 141 | tagctcgatatcgctcttg |
| | RAB11A-Sense 2 | SEQ ID NO: 142 | caagagcgatatcgagcta |
| siRNA 51 | RAB11A-Antisense 3 | SEQ ID NO: 143 | tgttctgacagcactgcac |
| | RAB11A-Sense 3 | SEQ ID NO: 144 | gtgcagtgctgtcagaaca |
| siRNA 52 | RAB11A-Antisense 4 | SEQ ID NO: 145 | aaacaatgcggtaaatctc |
| | RAB11A-Sense 4 | SEQ ID NO: 146 | gagatttaccgcattgttt |
| siRNA 53 | RERGL-Antisense 1 | SEQ ID NO: 147 | gattccacagctcttttac |
| | RERGL-Sense 1 | SEQ ID NO: 148 | gtaaaagagctgtggaatc |
| siRNA 54 | RERGL-Antisense 2 | SEQ ID NO: 149 | ctactaaacagatttcctt |
| | RERGL-Sense 2 | SEQ ID NO: 150 | aaggaaatctgtttagtag |
| siRNA 55 | RERGL-Antisense 3 | SEQ ID NO: 151 | ttctccaatgaatcgctta |
| | RERGL-Sense 3 | SEQ ID NO: 152 | taagcgattcattggagaa |
| siRNA 56 | RERGL-Antisense 4 | SEQ ID NO: 153 | aataacaaacccatctgcc |
| | RERGL-Sense 4 | SEQ ID NO: 154 | ggcagatgggtttgttatt |
| siRNA 57 | SH3PXD2A-Antisense 1 | SEQ ID NO: 155 | ttcaatgggaaacttatcc |
| | SH3PXD2A-Sense 1 | SEQ ID NO: 156 | ggataagtttcccattgaa |
| siRNA 58 | SH3PXD2A-Antisense 2 | SEQ ID NO: 157 | taaggaggagttccgtcg |
| | SH3PXD2A-Sense 2 | SEQ ID NO: 158 | cgacggaactcctcctta |
| siRNA 59 | SH3PXD2A-Antisense 3 | SEQ ID NO: 159 | tctcttcacagctacgtcc |
| | SH3PXD2A-Sense 3 | SEQ ID NO: 160 | ggacgtagctgtgaagaga |
| siRNA 60 | SH3PXD2A-Antisense 4 | SEQ ID NO: 161 | acatctttgaggttattgt |
| | SH3PXD2A-Sense 4 | SEQ ID NO: 162 | acaataacctcaaagatgt |
| siRNA 61 | SLC16A10-Antisense 1 | SEQ ID NO: 163 | tttccgacaaccaaatagg |
| | SLC16A10-Sense 1 | SEQ ID NO: 164 | cctatttggttgtcggaaa |
| siRNA 62 | SLC16A10-Antisense 2 | SEQ ID NO: 165 | aaacgcaaagggaacaacg |
| | SLC16A10-Sense 2 | SEQ ID NO: 166 | cgttgttccctttgcgttt |
| siRNA 63 | SLC16A10-Antisense 3 | SEQ ID NO: 167 | atccgctacctccactctc |
| | SLC16A10-Sense 3 | SEQ ID NO: 168 | gagagtggaggtagcggat |
| siRNA 64 | SLC16A10-Antisense 4 | SEQ ID NO: 169 | actaactcaaaggctatgg |
| | SLC16A10-Sense 4 | SEQ ID NO: 170 | ccatagcctttgagttagt |
| siRNA 65 | SLC22A25-Antisense 1 | SEQ ID NO: 171 | ttatggaaggaattagttc |
| | SLC22A25-Sense 1 | SEQ ID NO: 172 | gaactaattccttccataa |
| siRNA 66 | SLC22A25-Antisense 2 | SEQ ID NO: 173 | gaacccagcagcgatgatc |
| | SLC22A25-Sense 2 | SEQ ID NO: 174 | gatcatcgctgctgggttc |
| siRNA 67 | SLC22A25-Antisense 3 | SEQ ID NO: 175 | tatgtccaatactagcagc |
| | SLC22A25-Sense 3 | SEQ ID NO: 176 | gctgctagtattggacata |
| siRNA 68 | SLC22A25-Antisense 4 | SEQ ID NO: 177 | gaattggtgagttatccac |
| | SLC22A25-Sense 4 | SEQ ID NO: 178 | gtggataactcaccaattc |
| siRNA 69 | TTR-Antisense 1 | SEQ ID NO: 179 | aagatttggtgtctatttc |
| | TTR-Sense 1 | SEQ ID NO: 180 | gaaatagacaccaaatctt |
| siRNA 70 | TTR-Antisense 2 | SEQ ID NO: 181 | acaaattcctcctcagttg |
| | TTR-Sense 2 | SEQ ID NO: 182 | caactgaggaggaatttgt |
| siRNA 71 | TTR-Antisense 3 | SEQ ID NO: 183 | cctttctgaacacatgcac |
| | TTR-Sense 3 | SEQ ID NO: 184 | gtgcatgtgttcagaaagg |
| siRNA 72 | TTR-Antisense 4 | SEQ ID NO: 185 | ataccacctctgcatgctc |
| | TTR-Sense 4 | SEQ ID NO: 186 | gagcatgcagaggtggtat |
| siRNA 73 | TPRG1-Antisense 1 | SEQ ID NO: 187 | ttatcttgaactgctatcc |
| | TPRG1-Sense 1 | SEQ ID NO: 188 | ggatagcagttcaagataa |
| siRNA 74 | TPRG1-Antisense 2 | SEQ ID NO: 189 | tatcccattgttacagact |
| | TPRG1-Sense 2 | SEQ ID NO: 190 | agtctgtaacaatgggata |
| siRNA 75 | TPRG1-Antisense 3 | SEQ ID NO: 191 | acttccgtgagatataagg |
| | TPRG1-Sense 3 | SEQ ID NO: 192 | ccttatatctcacggaagt |
| siRNA 76 | TPRG1-Antisense 4 | SEQ ID NO: 193 | ttcctctcacaatatcgac |
| | TPRG1-Sense 4 | SEQ ID NO: 194 | gtcgatattgtgagaggaa |

A = adenine; T = uracil; C= cytosine; G = guanine

### Double-Stranded RNAs

A double-stranded RNA (dsRNA) molecule of the disclosure is a double-stranded nucleic acid molecule made of DNA, RNA, or both DNA and RNA (e.g., a chimeric) nucleosides that are complementary to an mRNA transcript (e.g., a transcript referenced in Table 1) encoding a target, or a portion or variant thereof, and prevents translation of the mRNA into a protein. Typically, a dsRNA molecule is longer than an siRNA molecule and are processed within a cell to form an siRNA molecule.

The antisense strand of the siRNA molecule is then incorporated into RISC, which, upon siRNA hybridization to a target mRNA (e.g., an mRNA transcript referenced in Table 1), will cleave the target mRNA, thereby inactivating the target mRNA and resulting in reduced mRNA and protein levels of the target. In some instances, a dsDNA may prevent translation of the mRNA into a protein via translation repression mechanisms.

In some embodiments, a dsRNA molecule of the disclosure may include a sense strand and an antisense strand, each containing a nucleotide sequence of about 25 to about 5,000 nucleotides in length, or longer.

It is within the scope of the disclosure that any length, known and previously unknown in the art, may be employed for the current invention.

In some embodiments, a dsRNA molecule of the disclosure contains a sequence complementary to at least 25 contiguous nucleotides set forth within at least one of the mRNA transcripts referenced in Table 1 or a variant thereof.

In some embodiments, the sense and antisense strands of a dsRNA molecule of the disclosure are completely complementary to one another. In some embodiments, the sense and antisense strands of a dsRNA molecule of the disclosure are completely complementary to the extent that their lengths overlap with one another. Depending on the sequence of the sense and antisense strand, complementarity need not be complete or perfect, which means that the sense and antisense strand are not 100% base-paired due to mismatches. One or more mismatches (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) may be present within the dsRNA molecule without impacting the dsRNA's ability to reduced expression of a target's mRNA (e.g., a transcript referenced in Table 1 or variant thereof) and/or protein (e.g., a protein referenced in Table 1).

The nucleotide sequence of a dsRNA molecule of the disclosure may contain sufficient complementarity to a portion of a transcript (e.g., a transcript referenced in Table 1 or variant thereof) encoding a target such that the dsRNA molecule can hybridize with one or more transcripts encoding the target. In some embodiments, the dsRNA molecule is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to one or more transcripts encoding a target (e.g., a transcript referenced in Table 1 or variant thereof), or a portion thereof. In some embodiments, the dsRNA molecule is 100% complementary to one or more transcripts encoding a target (e.g., a transcript referenced in Table 1 or variant thereof), or a portion thereof.

In some embodiments, the nucleotide sequence of a dsRNA molecule of the disclosure may contain sufficient complementarity to an exon sequence within one or more transcripts (e.g., a transcript listed in Table 1) encoding a target, or a portion thereof. In some embodiments, the nucleotide sequence of a dsRNA molecule may contain sufficient complementarity to an intron sequence within one or more transcripts (e.g., a transcript listed in Table 1) encoding a target, or a portion thereof. In some embodiments, a dsRNA molecule of the disclosure may contain sufficient complementarity to a pre-mRNA transcript or an mRNA transcript (e.g., a transcript listed in Table 1 or variant thereof) encoding a target described herein (e.g., a target listed in Table 1).

For any of the methods described herein, different dsRNA molecules of the disclosure can be combined for decreasing one or more transcripts (e.g., a transcript listed in Table 1) of one or more targets described herein (e.g., one or more targets listed in Table 1). A combination of two or more dsRNA molecules may be used in a method of the disclosure, such as two different dsRNA molecules, three different dsRNA molecules, four different dsRNA molecules, five different dsRNA molecules, or more, inhibiting the same target transcript (e.g., a transcript referenced in Table 1 or variant thereof). Alternatively, two different dsRNA molecules, three different dsRNA molecules, four different dsRNA molecules, five different dsRNA molecules, or more, inhibiting different target transcripts (e.g., two or more transcripts referenced in Table 1 or variants thereof) may be used in a method of the disclosure.

### MicroRNAs

A microRNA (miRNA) molecule of the disclosure is a short, ss nucleic acid molecules made of DNA, RNA, or both DNA and RNA (e.g., a chimeric) nucleosides that are complementary to an mRNA transcript (e.g., a transcript referenced in Table 1) encoding a target, or a portion or a variant thereof, and prevents translation of the mRNA into a protein. Once a miRNA molecule enters a cell, it is incorporated into RISC, which, upon miRNA hybridization to a target mRNA (e.g., an mRNA transcript referenced in Table 1), will cleave the target mRNA, thereby inactivating the target mRNA and resulting in reduced mRNA and protein levels of the target.

In some embodiments, a miRNA molecule of the disclosure may include a nucleotide sequence of about 6 to about 30 nucleotides in length (e.g., 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or 31 nucleotides in length).

In some embodiments, a miRNA molecule of the disclosure may include a nucleotide sequence of 6 to 30 nucleotides in length (e.g., 6, 7, 8, 9, 10, 11, 12, 13, 14, 15, 16, 17, 18, 19, 20, 21, 22, 23, 24, 25, 26, 27, 28, 29, or 30 nucleotides in length).

It is within the scope of the disclosure that any length, known and previously unknown in the art, may be employed for the current invention.

In some embodiments, a miRNA molecule of that disclosure contains a sequence complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides set forth within at least one of the mRNA transcripts referenced in Table 1 or a variant thereof.

The nucleotide sequence of the miRNA molecule may contain sufficient complementarity to a portion of a transcript (e.g., a transcript referenced in Table 1 or variant thereof) encoding a target such that the miRNA molecule can hybridize with one or more transcripts encoding the target. In some embodiments, the miRNA molecule is at least 70%, at least 75%, at least 80%, least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to one or more transcripts encoding the target (e.g., a transcript referenced in Table 1 or variants thereof), or a portion thereof. In some embodiments, the miRNA molecule is 100% complementary to a transcript encoding the target (e.g., a transcript referenced in Table 1 or a variant thereof), or a portion thereof.

In some embodiments, the nucleotide sequence of a miRNA molecule of the disclosure may contain sufficient complementarity to an exon sequence within one or more transcripts (e.g., a transcript listed in Table 1) encoding a target, or a portion thereof. In some embodiments, the nucleotide sequence of a miRNA molecule may contain sufficient complementarity to an intron sequence within one or more transcripts encoding a target, or a portion thereof. In some embodiments, a miRNA molecule of the disclosure may contain sufficient complementarity to a pre-mRNA transcript or an mRNA transcript encoding any one of the targets listed in Table 1 (e.g., a transcript referenced in Table 1 or variant thereof).

For any of the methods described herein, different miRNA molecules of the disclosure can be combined for decreasing one or more transcripts (e.g., a transcript listed in Table 1) of one or more targets described herein (e.g., one or more targets listed in Table 1). A combination of two or more miRNA molecules may be used in a method of the invention, such as two different miRNA molecules, three different miRNA molecules, four different miRNA molecules, five different miRNA molecules, or more, inactivating the same target transcript (e.g., a transcript referenced in Table 1 or variant thereof). Alternatively, two different miRNA molecules, three different miRNA molecules, four different miRNA molecules, five different miRNA molecules, or more, inactivating different target transcripts (e.g., two or more transcripts referenced in Table 1 or variants thereof) may be used in a method of the invention.

### Short Hairpin RNAs

A short hairpin RNA (shRNA) molecule of the disclosure is a ss or ds nucleic acid molecule made of DNA, RNA, or both DNA and RNA (e.g., a chimeric) nucleosides that are complementary to an mRNA transcript (e.g., a transcript referenced in Table 1) encoding a target, or a portion or variant thereof, and prevents translation of the mRNA into a protein. Once a shRNA molecule enters a cell, it is incorporated into a RISC, which, upon shRNA hybridization to a target mRNA (e.g., an mRNA transcript referenced in Table 1), the RISC complex will cleave the target mRNA, thereby inactivating the target mRNA and resulting in reduced mRNA and protein levels of the target.

In some embodiments, an shRNA molecule of the disclosure may include a nucleotide sequence of about 60 to about 100 nucleotides in length (e.g., 50, about 55, about 60, about 65, about 70, about 75, about 80, about 85, about 90, about 95, about 100, about 105, or 110 nucleotides in length).

In some embodiments, an shRNA molecule of the disclosure may include a nucleotide sequence of 60 to 100 nucleotides in length (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length).

In some embodiments, an shRNA molecule of the disclosure may contain a variable hairpin loop structure and a stem sequence. In some embodiments the stem sequence may be 10 to 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length). In some embodiments, the hairpin size is between 4 to 50 nucleotides in length (e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), although the loop size may be larger without significantly affecting silencing activity. An shRNA molecule of the disclosure may contain mismatches, for example G-U mismatches between two strands of the shRNA stem without decreasing potency. In some embodiments, an shRNA molecule is designed to include one or several G-U pairings in the hairpin stem to stabilize hairpins during propagation in bacteria, for example.

The nucleotide sequence of an shRNA molecule of the disclosure may contain sufficient complementarity to a portion of a transcript (e.g., a transcript referenced in Table 1 or a variant thereof) encoding a target such that the shRNA molecule can hybridize with one or more transcripts of the target. In some embodiments, the shRNA molecule is at least 70%, at least 75%, at least 80%, at least 85%, at least 90%, at least 95%, at least 96%, at least 97%, at least 98%, or at least 99% complementary to one or more transcripts encoding a target (e.g., a transcript referenced in Table 1 or a variant thereof), or a portion thereof. In some embodiments, the shRNA molecule is 100% complementary to one or more transcripts encoding a target (e.g., a transcript referenced in Table 1 or a variant thereof), or a portion thereof.

In some embodiments, the nucleotide sequence of an shRNA molecule of the disclosure may contain sufficient complementarity to an exon sequence within one or more transcripts (e.g., a transcript listed in Table 1 encoding a target, or a portion thereof. In some embodiments, the nucleotide sequence of an shRNA molecule may contain sufficient complementarity to an intron sequence within one or more transcripts (e.g., a transcript listed in Table 1) encoding a target, or a portion thereof. In some embodiments, an shRNA molecule of the disclosure may contain sufficient complementarity to a pre-mRNA transcript or an mRNA transcript (e.g., a transcript listed in Table 1 or a variant thereof) encoding a target described herein (e.g., a target listed in Table 1).

For any of the methods described herein, different shRNA molecules can be combined for decreasing one or more transcripts (e.g., a transcript listed in Table 1) encoding one or more targets described herein (e.g., one or more targets listed in Table 1). A combination of two or more shRNA molecules may be used in a method of the invention, such as two different shRNA molecules, three different shRNA molecules, four different shRNA molecules, five different shRNA molecules, or more, for reducing the same target transcript (e.g., a transcript referenced in Table 1 or a variant thereof). Alternatively, two different shRNA molecules, three different shRNA molecules, four different shRNA molecules, five different shRNA molecules, or more, for reducing different target transcripts (e.g., two or more transcripts referenced in Table 1 or variants thereof) may be used in a method of the invention.

### Antisense Oligonucleotides

An antisense oligonucleotide (ASO) of the disclosure is a ss nucleic acid molecule containing DNA nucleosides that are complementary to an mRNA transcript (e.g., a transcript referenced in Table 1) encoding a target, or a portion or variant thereof, and prevents translation of the mRNA into a protein. Upon hybridization of an ASO to a target mRNA (e.g., an mRNA transcript referenced in Table 1), RNase H will degrade the mRNA by hydrolyzation, resulting in reduced mRNA and protein levels of the target.

In some embodiments, an ASO of the disclosure may include a nucleotide sequence of about 12 to about 50 nucleotides in length (e.g., 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, or 51 nucleotides in length).

In some embodiments, an ASO of the disclosure may include a nucleotide sequence of 12 to 50 nucleotides in length (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length).

It is within the scope of the disclosure that any length, known and previously unknown in the art, may be employed for the current invention.

In some embodiments, an ASO of the disclosure contains a sequence complementary to at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 contiguous nucleotides set forth within at least one of the mRNA transcripts referenced in Table 1 or a variant thereof.

The nucleotide sequence of an ASO of the disclosure may contain sufficient complementarity to a portion of a transcript (e.g., a transcript referenced in Table 1 or variant thereof) encoding a target such that the ASO can hybridize with one or more transcripts encoding the target. In some embodiments, the ASO is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to one or more transcripts encoding a target (e.g., a transcript referenced in Table 1 or variants thereof), or a portion thereof. In some embodiments, the ASO is 100% complementary to one or more transcripts encoding a target (e.g., a transcript referenced in Table 1, or variants thereof), or a portion thereof.

In some embodiments, the nucleotide sequence of an ASO of the disclosure may contain sufficient complementarity to an exon sequence within one or more transcripts (e.g., a transcript listed in Table 1) encoding a target, or a portion thereof. In some embodiments, the nucleotide sequence of an ASO may contain sufficient complementarity to an intron sequence within one or more transcripts encoding a target, or a portion thereof. In some embodiments, an ASO of the disclosure may contain sufficient complementarity to a pre-mRNA transcript or an mRNA transcript encoding any one of the targets listed in Table 1 (e.g., a transcript referenced in Table 1 or a variant thereof). In some embodiments, the ASO is IONIS—FB-LRx, which targets CFB.

For the methods described herein, different ASOs can be combined for decreasing one or more transcripts (e.g., a transcript listed in Table 1) of one or more targets described herein (e.g., one or more targets listed in Table 1). A combination of two or more ASOs may be used in a method of the invention, such as two different ASOs, three different ASOs, four different ASOs, or five different ASOs for degrading the same target transcript (e.g., a transcript referenced in Table 1 or variant thereof). Alternatively, two different ASOs, three different ASOs, four different ASOs, or five different ASOs for degrading different target transcripts (e.g., two transcripts referenced in Table 1 or variants thereof) may be used in a method of the invention.

### GapmeRs

A gapmeR of the disclosure is a single-stranded nucleic acid molecule containing an internal DNA region (i.e., a gap segment) flanked by one or two external RNA regions (i.e., wing segments). At a minimum, the gap segment contains a sequence complementary to an mRNA transcript (e.g., a transcript referenced in Table 1) encoding a target and, typically, the wing segments contain modified RNA; modified RNA (and DNA) is described further below. Upon the hybridization of a gapmeR to a target mRNA (e.g., an mRNA transcript encoding a target referenced in Table 1), RNase H will degrade the mRNA by hydrolyzation, resulting in reduced mRNA and protein levels of the target.

In some embodiments, a gapmeR of the disclosure may include a nucleotide sequence of about 10 to about 25 nucleotides in length (e.g., 9, about 10, about 11, about 12, about 13, about 14, about 15, about 16, about 17, about 18, about 19, about 20, about 21, about 22, about 23, about 24, about 25, or 26 nucleotides in length).

In some embodiments, a gapmeR of the disclosure may include a nucleotide sequence of 10 to 25 nucleotides in length (e.g., 10, 11, 12, 13, 14, 15, 16, 17, 18, 19, 20, 21, 22, 23, 24, or 25 nucleotides in length).

It is within the scope of the disclosure that any length, known and previously unknown in the art, may be employed for the current invention.

In some embodiments, a gapmeR of the disclosure contains at least one (e.g., 1 or 2) wing region. In some embodiments, the gapmeR contains one wing region located 5′ to the gap region. In some embodiments, the gapmeR contains one wing region located 3′ to the gap region. In some embodiments, the gapmeR contains two wing regions, one located 5′ to the gap region and the other located 3′ to the gap region.

In some embodiments, a gapmeR of the disclosure contains at least one (e.g., 1 or 2) wing region that is about 1 to about 7 nucleotides in length (e.g., about 1-8, about 1-7, about 1-6, about 1-5, about 1-4, about 1-3, or about 1-2 nucleotides in length). In some embodiments, the gapmeR contains at least one (e.g., 1 or 2) wing region that is 1 nucleotide in length. In some embodiments, the gapmeR contains at least one (e.g., 1 or 2) wing region that is 2 nucleotides in length. In some embodiments, the gapmeR contains at least one (e.g., 1 or 2) wing region that is 3 nucleotides in length. In some embodiments, the gapmeR contains at least one (e.g., 1 or 2) wing region that is 4 nucleotides in length. In some embodiments, the gapmeR contains at least one (e.g., 1 or 2) wing region that is 5 nucleotides in length.

In some embodiments, the gapmeR contains at least one (e.g., 1 or 2) wing region that is 6 nucleotides in length. In some embodiments, the gapmeR contains at least one (e.g., 1 or 2) wing region that is 7 nucleotides in length. In some embodiments, the gapmeR contains at least one (e.g., 1 or 2) wing region that is 8 nucleotides in length.

In some embodiments, a gapmeR of the disclosure contains one gap region. In some embodiments, the gapmeR contains one gap region located 5′ to the wing region. In some embodiments, the gapmeR contains one gap region located 3′ to the wing region. In some embodiments, the gapmeR contains one gap region flanked by two wing regions.

In some embodiments, a gapmeR of the disclosure contains at least one gap region that is about 8 to about 24 nucleotides in length (e.g., about 8-24, about 8-23, about 8-22, about 8-21, about 8-20, about 8-19, about 8-18, about 8-17, about 8-16, about 8-15, about 8-14, about 8-13, about 8-12, about 8-10, or about 8-9 nucleotides in length).

The nucleotide sequence of the gapmeR may contain sufficient complementarity to a portion of a transcript (e.g., a transcript referenced in Table 1 or variant thereof) encoding a target such that the gapmeR can hybridize with one or more transcripts encoding the target. In some embodiments, the gapmeR is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to one or more transcripts encoding target (e.g., a transcript referenced in Table 1 or variants thereof), or a portion thereof. In some embodiments, the gapmeR is 100% complementary to one or more transcripts encoding a target, or a portion thereof.

In some embodiments, the nucleotide sequence of a gapmeR of the disclosure may contain sufficient complementarity to an exon sequence within one or more transcripts (e.g., a transcript listed in Table 1 encoding a target, or a portion thereof. In some embodiments, the nucleotide sequence of a gapmeR may contain sufficient complementarity to an intron sequence within one or more transcripts encoding a target, or a portion thereof. In some embodiments, the nucleotide sequence of a gapmeR of the disclosure may contain sufficient complementarity to a pre-mRNA transcript or an mRNA transcript encoding any one of the targets listed in Table 1 (e.g., a transcript referenced in Table 1 or a variant thereof).

For the methods described herein, different gapmeRs can be combined for decreasing one or more transcripts (e.g., a transcript listed in Table 1) encoding one or more targets described herein (e.g., one or more targets listed in Table 1). A combination of two or more gapmeRs may be used in a method of the invention, such as two different gapmeRs, three different gapmeRs, four different gapmeRs, five different gapmeRs, or more, degrading the same target transcript (e.g., a transcript referenced in Table 1 or a variant thereof). Alternatively, two different gapmeRs, three different gapmeRs, four different gapmeRs, or five different gapmeRs degrading different target transcripts (e.g., two transcripts referenced in Table 1 or variants thereof) may be used in a method of the invention.

### Protein-Encoding RNAs

In some embodiments, the agent is an RNA molecule (e.g., an mRNA) encoding a protein that reduces the expression or activity of one or more of the targets listed in Table 1. In some embodiments, the RNA molecule (e.g., an mRNA) encodes a protein that reduces the expression or activity of one or more of the targets listed in Table 1. In some embodiments, the protein is soluble or membrane bound. In some embodiments, the RNA molecule encodes a protein or peptide listed in Table 3. In some embodiments, the agent is an RNA molecule (e.g., an mRNA) encoding RFP26, which inhibits RAB11A.

### Soluble Proteins

In some embodiments, the agent is a soluble protein or a peptide that reduces the expression or activity of one or more of the targets listed in Table 1 (e.g., a protein or peptide listed in Table 3). An exemplary protein inhibitor is peptide inhibitor RFP26, which inhibits target RAB11A.

In some embodiments, the soluble protein is an agent that disrupts binding of the target to a binding partner. For example, the soluble protein may be a variant of the target that lacks the ability to bind to a binding partner (e.g., a variant of the target in which the binding domain has been mutated or deleted), or a variant of a binding partner of the target that lacks the ability to bind to the target (e.g., a variant of the binding partner in which the binding domain has been mutated or deleted). In some embodiments, the soluble protein is an agent that disrupts downstream signaling, such as a variant of a transmembrane target that lacks the intracellular domain but retains the ability to bind to extracellular binding partners or a variant of a target in which one or more signaling domains have been mutated.

### Small Molecule Inhibitors

In some embodiments, the agent is a small molecule inhibitor that reduces the expression or activity of one or more of the targets listed in Table 1. In some embodiments, the small molecule inhibits ABCB4 (e.g., cyclosporine A, valspodar, verapamil, vinblastine, paclitaxel, or itraconazole).

In some embodiments, the small molecule inhibitor is an antagonist that binds directly to the target to reduce or inhibit its function or activity. In some embodiments, the small molecule antagonist is selective for the target and does not exhibit substantial binding to other proteins.

Small molecules include organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. An exemplary small molecule inhibitor that can be used to inhibit a target described herein is the CFB inhibitor LNP023 or iptacopan (Novartis AG, Basel, Switzerland), which is described elsewhere, such as, e.g., WO2022234541, WO2023166487, WO2022264101, and WO2023037218, each of which is hereby incorporated by reference. Additional exemplary small molecule inhibitors that can be used to inhibit a target described herein are the ACMSD inhibitor TES-991 and the ABCB4 inhibitors cyclosporine A, valspodar, verapamil, vinblastine, paclitaxel, and itraconazole. Additional small molecule inhibitors can also be identified through screening based on their ability to reduce or inhibit the function or signaling of a target listed in Table 1.

### Antibodies

In some embodiments, the inhibitory agent is an inhibitory antibody (e.g., mAb), that reduces the expression or activity of one or more of the targets listed in Table 1. Exemplary antibodies are SAR-443809 and NM-8074, each of which bind target CFB.

In some embodiments, the antibody is against one or more of the targets listed in Table 1, or an antigen binding fragment thereof, that binds to a target listed in Table 1. In some embodiments, the antibody is an antibody against the target, or an antigen binding fragment thereof, that reduces or inhibits the function of the target. The antibody can have one or more of the following functional properties: prevent the target from binding to a binding partner (e.g., sterically hinder the binding of the target to a binding partner), sequester a soluble target, and/or reduce or inhibit the activity and/or function of the target. Antibodies having one or more of these functional properties are routinely screened and selected once the desired functional property is identified herein (e.g., by screening of phage display or other antibody libraries).

The making and use of antibodies against a target antigen (e.g., against a target described herein (e.g., a protein listed in Table 1)) is known in the art (see, for example, Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques).

### Gene Editing-Related Agents

In some embodiments, the agent is a component of a gene editing system that reduces the expression or activity of one or more targets listed in Table 1. For example, the gene editing-mediated agent introduces an alteration (e.g., insertion, deletion (e.g., knockout), translocation, inversion, single point mutation, or other mutation) in a gene (e.g., a genomic locus or an mRNA transcript) encoding a target (e.g., a target listed in Table 1). Exemplary gene editing systems (e.g., base editing, prime editing, or homology-directed repair (HDR) systems) may include the zinc finger nucleases (ZFNs), Transcription Activator-Like Effector-based Nucleases (TALENs), and the clustered regulatory interspaced short palindromic repeat (CRISPR) system listed in Table 2. ZFNs, TALENs, and CRISPR-based methods are described, e.g., in Gaj et al. Trends Biotechnol. 31.7:397-405, 2013.

CRISPR refers to a set of (or system comprising a set of) clustered regularly interspaced short palindromic repeats. A CRISPR system refers to a system derived from CRISPR and Cas (a CRISPR-associated protein) or another nuclease that can be used to silence or mutate a gene (e.g., a genomic locus or an mRNA transcript) encoding a target described herein. The CRISPR system is a naturally occurring system found in bacterial and archeal genomes. The CRISPR locus is made up of alternating repeat and spacer sequences. In naturally occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature. 482:331, 2012. For example, such modification of the system includes introducing into a eukaryotic cell a plasmid containing a specifically designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science. 327:167, 2010; Makarova et al., Biology Direct. 1:7, 2006; Pennisi, Science. 341:833, 2013. In some examples, the CRISPR system includes the Cas9 protein, a nuclease that cuts on both strands of the DNA. See, e.g., Id.

In some embodiments, the CRISPR system may include one of the following nucleases that cuts both strands of a DNA sequence (e.g., a DNA sequence encoding a target referenced in Table 1): Cas3, Cas12a (e.g., AsCas12a, LbCas12a), Cas12b, Cas12c, Cas12d, Cas12e, Cas14, Cas12h,Cas12i, and Cas12j. In other embodiments, the CRISPR system includes one of the following nucleases that cuts a strand of an mRNA sequence (e.g., an mRNA transcript referenced in Table 1, or a variant thereof): Cas13a (LbaCas13, LbuCas13, LwaCas13), Cas13b (e.g., CccaCas13b, PsmCas13b), and Cas12g.

In some embodiments, in a CRISPR system for use described herein, e.g., in accordance with one or more methods described herein, the spacers of the CRISPR are derived from a target gene sequence, e.g., from a gene sequence encoding a target (e.g., a target listed in Table 1).

In some embodiments, the gene editing-mediated agent includes a guide RNA (gRNA) or prime-editing gRNA (pegRNA) for use in a clustered regulatory interspaced short palindromic repeat (CRISPR) system for gene editing (e.g., HDR-mediated editing, prime editing, or base editing). In embodiments, the gene editing-mediated agent comprises a zinc finger nuclease (ZFN), or an mRNA encoding a ZFN, that targets (e.g., cleaves) a nucleic acid sequence (e.g., DNA sequence) encoding a target (e.g., a target listed in Table 1). In embodiments, the gene editing-mediated agent comprises a TALEN, or an mRNA encoding a TALEN, that targets (e.g., cleaves) a nucleic acid sequence (e.g., DNA sequence) encoding a target (e.g., a target listed in Table 1).

In some examples, the gRNA can be used in a CRISPR system to engineer an alteration in a gene (e.g., a gene encoding a target). In other examples, the ZFN and/or TALEN can be used to engineer an alteration in a gene (e.g., a gene encoding a target). Exemplary alterations include frameshift insertions or deletions (indels) (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) the target (e.g., a target listed in Table 1), e.g., the alteration is a negative regulator of function.

In certain embodiments, the CRISPR system is used to edit (e.g., to add or delete a base pair) a target gene, e.g., a genomic locus or mRNA transcript encoding a target (e.g., a target listed in Table 1). In other embodiments, the CRISPR system is used to introduce a premature stop codon, e.g., thereby decreasing the expression or reducing function of a target gene (e.g., a genomic locus or an mRNA transcript). In yet other embodiments, the CRISPR system is used to turn off a target gene in a reversible manner, e.g., similarly to RNA interference. In embodiments, the CRISPR system is used to direct Cas to a promoter of a target gene, e.g., a genomic locus encoding a target (e.g., a target listed in Table 1), thereby blocking an RNA polymerase sterically.

In some embodiments, a CRISPR system can be generated to edit a gene encoding a target (e.g., a target listed in Table 1) using technology described in, e.g., U.S. Publication No. 20140068797; Cong, Science 339: 819, 2013; Tsai, Nature Biotechnol. 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding a target (e.g., a target listed in Table 1). In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In any aforementioned CRISPR system, suitable gRNAs include at least one crispr RNA (crRNA) region to enable specificity in every CRISPR reaction. Suitable gRNAs may also include a trans-activating crRNA (tracrRNA) region fused to or hybridized with the crRNA. Additionally, suitable gRNAs may contain multiple endoribonuclease recognition sites (e.g., Csy4) for multiplex processing. If an endoribonuclease recognition site is introduced between neighboring gRNA sequences, more than one gRNA can be transcribed in a single expression cassette. Direct repeats can also serve as endoribonuclease recognition sites for multiplex processing.

A gRNA may contain a spacer sequence containing a plurality of bases and complementarity to a protospacer sequence in a target DNA (e.g., a DNA sequence encoding a target listed in Table 1) or mRNA (e.g., see Table 1) sequence. The gRNA spacer sequence may be 50%, 55%, 60%, 65%, 70%, 75%, 80%, 85%, 90%, 91%, 92%, 93%, 94% 95%, 86%, 97%, 98%, 99%, or 100% complementary to its intended target sequence (e.g., a target listed in Table 1).

A gRNA may contain one or more modified or non-naturally occurring nucleoside or nucleotide, and/or internucleoside linkage. In some embodiments, the gRNA may contain a locked nucleic acid (LNA), a bridged nucleic acid (BNA), and/or a peptide nucleic acid (PNA). By way of further example, a modified nucleic acid molecule may contain a modified or non-naturally occurring nucleoside, nucleotide, and/or internucleoside linkage, such as a 2′-O-methyl (2′-O-Me) modified nucleoside, a 2′-fluoro (2′-F) modified nucleoside, and a phosphorothioate (PS) bond (or any other nucleic acid molecule modifications described herein).

### Modifications to Nucleic Acid Molecules

It is contemplated that any of the nucleic acid molecules described herein (e.g., inhibitory nucleic acid molecules, protein-encoding RNAs, gRNAs) may be used in the methods disclosed herein in an unmodified or in a modified form. Unmodified nucleic acid molecules contain nucleobases that include the purine bases adenine (A) and guanine (G), and the pyrimidine bases thymine (T), cytosine (C), and uracil (U). Modified nucleic acid molecules are described in more detail below.

Modifications may be achieved by systematically adding or removing linked nucleosides to generate longer or shorter sequences.

Modifications may be achieved by incorporating, for example, one or more alternative nucleosides, alternative 2′ sugar moieties, and/or alternative internucleoside linkages. Typically, these types of modifications are introduced to optimize the molecule's efficacy or biophysical properties (e.g., increasing serum stability or circulating half-life, increasing thermal stability, enhancing transmembrane delivery, reduce immunogenicity, and/or targeting to a particular location or cell type). By way of example, a modified nucleotide such as a locked nucleic acid (LNA), a peptide nucleic acid (PNA), or a bridged nucleic acid (BNA) may be incorporated into any nucleic acid-based inhibitor described above. Further nucleic acid modifications are described below.

Modification may further be achieved by covalently or non-covalently conjugating a moiety (e.g., a targeting moiety, a hydrophobic moiety, a cell penetrating peptide, or a polymer) to the 5′ end and/or 3′ end of the inhibitory nucleic acid molecule, as described in more detail below.

### Nucleoside Modifications

Modification of the inhibitory nucleic acid molecules described herein include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The inhibitory nucleic acid molecules may also include nucleobases in which the purine or pyrimidine base is replaced with other heterocycles, for example 7-deaza-adenine, 7-deazaguanosine, 2-aminopyridine, and/or 2-pyridone. Further modification of the inhibitory nucleic acid molecules described herein may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

### Sugar Modifications

Modifications of the inhibitory nucleic acid molecules described herein may also include one or more of the following 2′ sugar modifications: 2′-O-methyl (2′-O-Me), 2′-methoxyethoxy (2′-O—CH2CH2OCH3, also known as 2′-O-(2-methoxyethyl) or 2′-MOE), 2′-dimethylaminooxyethoxy, i.e., a O(CH2)2ON(CH3)2 group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH2OCH2N(CH3)2. Other possible 2′-modifications that can modify the inhibitory nucleic acid molecules described herein include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C10 alkyl or C₂ to C10 alkenyl and alkynyl. Other potential sugar substituent groups include, e.g., aminopropoxy (—OCH2CH2CH2NH2), allyl (-CH2-CH═CH2), —O-allyl (—O—CH2-CH═CH2) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Internucleoside Linkage Modifications

Modifications of the inhibitory nucleic acid molecules described herein may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### Conjugates

Any of the inhibitory nucleic acid molecules described herein may be modified via the addition of an auxiliary moiety, e.g., a cell penetrating peptide (CPP), a polymer, a hydrophobic moiety, or a targeting moiety. The auxiliary moiety may be present as a 5′ terminal modification (e.g., covalently bonded to a 5′-terminal nucleoside), a 3′ terminal modification (e.g., covalently bonded to a 3′-terminal nucleoside), or an internucleoside linkage (e.g., covalently bonded to phosphate or phosphorothioate in an internucleoside linkage).

CPPs are known in the art (e.g., TAT or Arg8) (Snyder and Dowdy. Expert Opin. Drug Deliv. 2:43-51, 2005). Specific examples of CPPs are provided in WO2011157713, which is incorporated herein by reference in its entirety.

Inhibitory nucleic acid molecules of the disclosure may include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers.

An inhibitory nucleic acid molecule containing a hydrophobic moiety may exhibit superior cellular uptake, as compared to an inhibitory nucleic acid molecule lacking the hydrophobic moiety. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the nucleic acid backbone (e.g., 5′-terminus) of the inhibitory nucleic acid molecule.

A targeting moiety is selected based on its ability to target oligonucleotides of the invention to a desired or selected cell population that expresses the corresponding binding partner (e.g., either the corresponding receptor or ligand) for the selected targeting moiety. For example, an oligonucleotide of the invention could be targeted to hepatocytes expressing asialoglycoprotein receptor (ASGP-R) by selecting a targeting moiety containing N-acetylgalactosamine (GaINAc).

A targeting moiety may include one or more ligands (e.g., 1 to 9 ligands, 1 to 6 ligands, 1 to 3 ligands, 3 ligands, or 1 ligand). The ligand may target a cell expressing asialoglycoprotein receptor (ASGP-R), IgA receptor, HDL receptor, LDL receptor, or transferrin receptor. Non-limiting examples of the ligands include N-acetylgalactosamine, glycyrrhetinic acid, glycyrrhizin, lactobionic acid, lactoferrin, IgA, or a bile acid (e.g., lithocholyltaurine or taurocholic acid).

The ligand may be a small molecule, e.g., a small molecule targeting a cell expressing asialoglycoprotein receptor (ASGP-R). A non-limiting example of a small molecule targeting an asialoglycoprotein receptor is N-acetylgalactosamine. Alternatively, the ligand can be an antibody or an antigen-binding fragment or an engineered derivative thereof (e.g., Fcab or a fusion protein (e.g., scFv)).

### Pharmaceutical Compositions

The agents described herein may be formulated into various compositions (e.g., a pharmaceutical composition) for administration to a subject in a biologically compatible form suitable for administration in vivo. For example, the agents described herein may be administered in a suitable diluent, carrier, stabilizer, or excipient, and may further contain a preservative, e.g., to prevent the growth of microorganisms. Conventional procedures and ingredients for the selection and preparation of suitable compositions are described, for example, in Remington, J. P. The Science and Practice of Pharmacy, Easton, PA. Mack Publishers, 2012, 22nd ed. and in The United States Pharmacopeial Convention, The National Formulary, United States Pharmacopeial, 2015, USP 38 NF 33.

Mixtures of agents described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1,000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates and mammals.

Compositions containing an agent described herein may further include a second agent (e.g., a nucleic acid molecule to be expressed within a cell, a polypeptide, or a drug). For example, a second agent may be a blood pressure medication, steroid, or immunosuppressive agent. In some embodiments, the second agent is a statin. In some embodiments, the second agent (e.g., a statin) is administered in combination with an agent of the disclosure.

### Exosomes

In one embodiment, pharmaceutical compositions of an agent described herein include exosomes. Exosomes produced from cells can be collected from the culture medium by any suitable method. Typically, a preparation of exosomes can be prepared from cell culture or tissue supernatant by centrifugation, filtration, or combinations of these methods. For example, using standard methods, exosomes can be prepared by differential centrifugation, that is low speed (<20,000×g) centrifugation to pellet larger particles followed by high speed (>100,000×g) centrifugation to pellet exosomes, size filtration with appropriate filters (for example, 0.22 micrometer filter), gradient ultracentrifugation (for example, with sucrose gradient) or a combination of these methods. Exosomes are loaded with exogenous inhibitors, according to standard methods, for systemic delivery to a subject (e.g., a human).

### Liposomes

In one embodiment, pharmaceutical compositions of an agent described herein include liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. Liposomes are loaded with exogenous inhibitors, according to standard methods, for systemic delivery to a subject (e.g., a human).

### Lipid Nanoparticles

In one embodiment, pharmaceutical compositions of an agent described herein include lipid nanoparticles (LNPs). For example, the inhibitor, such as a 5′-inhibitor-Pro-CGG-1 and/or a 5′-inhibitor-Cys-GCA-27, may be formulated in a lipid nanoparticle such as those described in International Publication No. WO2012170930, herein incorporated by reference in its entirety. As a non-limiting example, LNP formulations may contain cationic lipids, distearoylphosphatidylcholine (DSPC), cholesterol, polyethylene glycol (PEG), R-3-[(w-methoxy poly(ethylene glycol)2000)carbamoyl)]-1,2-dimyristyloxl-propyl-3-amine (PEG-c-DOMG), distearoyl-rac-glycerol (DSG) and/or dimethylaminobutanoate (DMA). As a non-limiting example, 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypoly ethylene glycol) or PEG-DPG (1,2-Dipalmitoyl-sn-glycerol, methoxypolyethylene glycol). The cationic lipid may be selected from any lipid known in the art such as, but not limited to, (6Z,9Z,28Z,31Z)-heptatriacont-6,9,28,31-tetraene-19-yl 4-(dimethylamino)butanoate (DLin-MC3-DMA), 1,2-dilinoleyloxy-n,n-dimethyl-3-aminopropane (DLin-DMA), C 12-200, and N,N-dimethyl-2,2-di-(9Z,12Z)-9,12-octadecadien-1-yl-1,3-dioxolane-4-ethanamine (DLin-KC2-DMA). LNPs are loaded with exogenous therapeutic agents, according to standard methods, for systemic delivery to a subject (e.g., a human).

In some embodiments, compounds described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### Cationic Lipids and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N—(I-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylarmonium chloride (DODAC), N,N-dimethyl-2,3-di:aeyloxy)propylamine (DODMA), 1,2-Dioleoyl-3-Dimethylammoniurn-propane (DODAP), 1,2-Dioleoylcarbarnyl-3-Dimethylamrnonium.-propane (DOCDAP), 1,2-Dilineoyl-3-Dimethylammoniumn-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in, e.g., Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

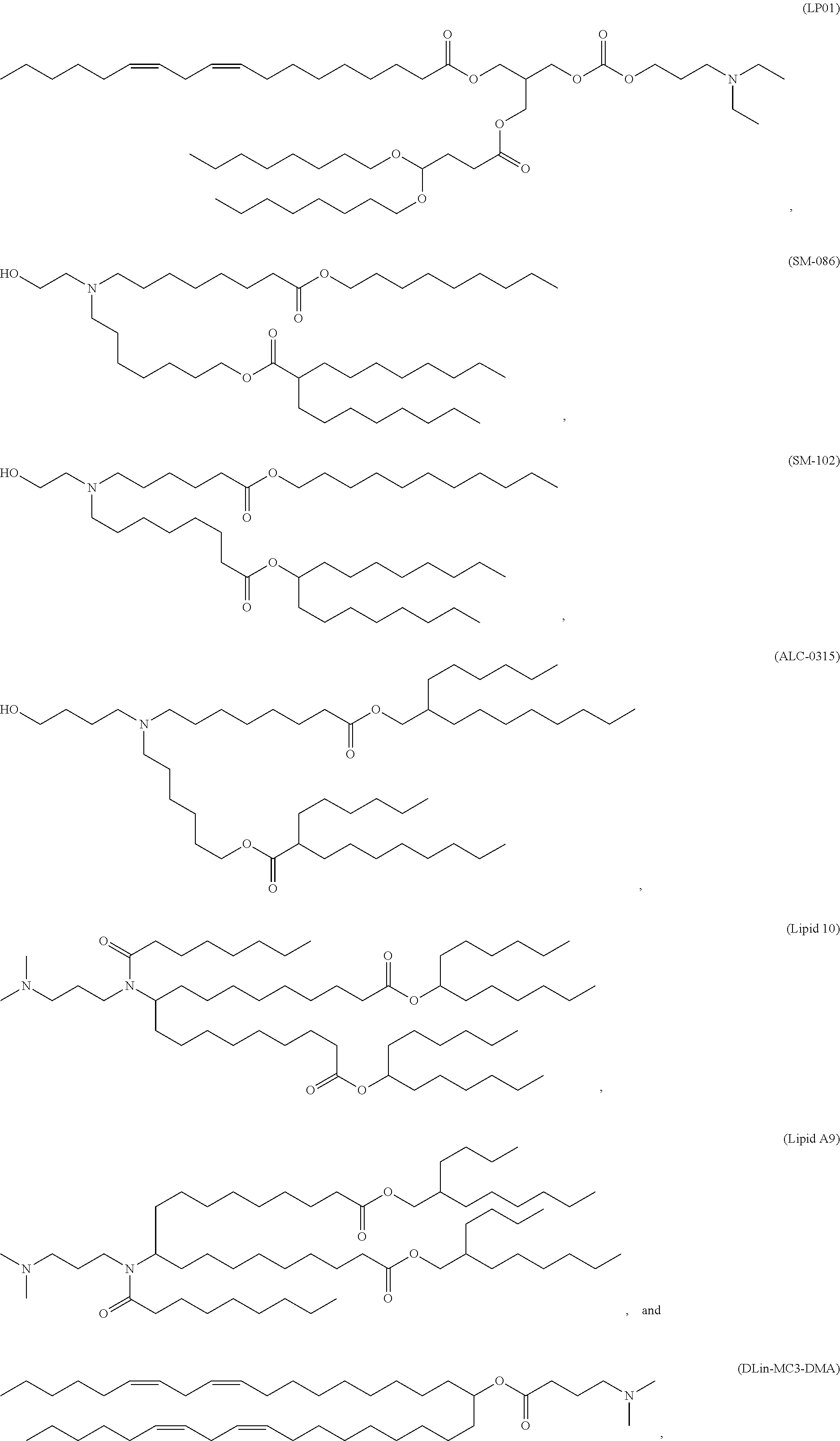

(see WO 20171004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater. 6: 1078-1094, 2021, https://doi.org/10.1038/s41578-021-00358-0 (e.g., L319, C12-200, and Dlin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; 1, 11 or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; 1, 11, 111, or IV of US 2012/01287670; I or II of US 2014/0200257; 1, 11, or III of US 2015/0203446; I or III of US 2015/0005363; I, IA, IB, IC, ID, II, IIA, IIB, IIC, IID, or III-XXIV of US 2014/0308304; of US 2013/0338210; 1, 11, 111, or IV of WO 2009/132131; A of US 2012/01011478; I or XXXV of US 2012/0027796; XIV or XVII of US 2012/0058144; of US 2013/0323269; I of US 2011/0117125; 1, 11, or III of US 2011/0256175; 1, 11, 111, IV, V, VI, VII, VIII, IX, X, XI, XII of US 2012/0202871; 1, 11, 111, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y—Z of US 2015/0064242; XVI, XVII, or XVIII of US 2013/0022649; 1, 11, or III of US 2013/0116307; 1, 11, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or 1-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Eygeris et al. (Nano Lett. (20)6:4543-4549, 2020), which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, 51imyristoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipids in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (Nano Lett. 20(6):4543-4549, 2020), incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethylenegly col-diacylglycerol (i.e., polyethylenegiycol diacyigly cerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristoylglycerol (C14), a PEG-dipalmitoylglycerol (C16), a PEG-disterylglycerol (C18), PEG-dilaurylglycarnide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DAG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DAG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-1, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

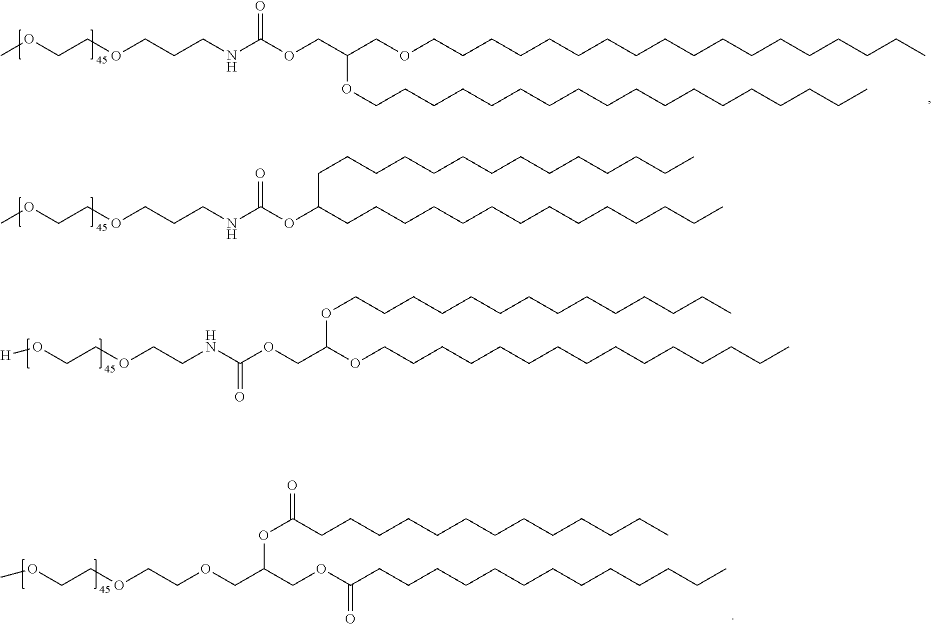

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther. 289:1128-1133, 1999; Gabizon et al. J Controlled Release. 53:275-279, 1998; Adlakha Hutcheon et al. Nat Biotechnol. 17:775-779, 1999); and Koning et al. Biochim Biophys Acta. 1420:153-167, 1999), which are incorporated herein by reference in their entirety.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a payload (e.g., an inhibitor of a target listed in Table 1; e.g., an siRNA listed in Table 4). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a payload (e.g., an inhibitor of a target listed in Table 1; e.g., an siRNA listed in Table 4) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a payload (e.g., an inhibitor of a target listed in Table 1; e.g., an siRNA listed in Table 3 or Table 4) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a payload (e.g., an inhibitor of a target listed in Table 1; e.g., an siRNA listed in Table 3 or Table 4) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a payload (e.g., an inhibitor of a target listed in Table 1; e.g., an siRNA listed in Table 3 or Table 4) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), O to 10% non-cationic-lipid, and 1-10% conjugated lipid (e.g., the PEGylated lipid).

In some embodiments, the lipid-based carrier comprises a payload (e.g., an inhibitor of a target listed in Table 1; e.g., an siRNA listed in Table 3 or Table 4) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a nucleic acid (e.g., an siRNA listed in Table 3 or Table 4); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a nucleic acid (e.g., an siRNA listed in Table 3 or Table 4); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated agent such as a nucleic acid; e.g., an siRNA listed in Table 3 or Table 4) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulations) overall lipid content can range from about 5 mg/ml to about 30 mg/mL. Nitrogen:phosphate ratios (N:P ratio) is evaluated at values between 0.1 and 100.

The efficiency of encapsulation of a payload such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### Carbohydrate Targeting Moieties

In some embodiments, a carbohydrate targeting moiety may form a complex with an inhibitor of a target listed in Table 1. The carbohydrate targeting moiety may, for example, comprise a saccharide, disaccharide, or polysaccharide. In some embodiments, the carbohydrate includes mannose, galactose, or glucose. In some embodiments, a carbohydrate moiety described herein comprises one or more monosaccharide moieties. In some embodiments, the one or more monosaccharide moieties comprises at least 3 carbon atoms (e.g., arranged in a linear, branched, or cyclic structure) and an oxygen, nitrogen, or sulfur atom, or a fragment or variant of a monosaccharide moiety comprising at least 3 carbon atoms (e.g., arranged in a linear, branched, or cyclic structure) and an oxygen, nitrogen, or sulfur atom. Each monosaccharide moiety or fragment or variant thereof may be a tetrose, pentose, hexose, or heptose. Each monosaccharide moiety or fragment or variant thereof may exist as an aldose, ketose, sugar alcohol, and, where appropriate, in the L or D form. Exemplary monosaccharide moieties may be amino sugars, N-acetylamino sugars, imino sugars, deoxysugars, or sugar acids. Carbohydrates may comprise individual monosaccharide moieties, or may further comprise a disaccharide, oligosaccharide (e.g., a trisaccharide, tetrasaccharide, pentasaccharide, hexasaccharide, heptasaccharide, octasaccharide), a polysaccharide, or combinations thereof. Exemplary carbohydrates include ribose, arabinose, lyxose, xylose, deoxyribose, ribulose, xylulose, glucose, galactose, mannose, gulose, idose, talose, allose, altrose, psicose, fructose, sorbose, tagatose, rhamnose, pneumose, quinovose, fucose, mannuheptulose, sedoheptulose, galactosamine, mannosamine, glucosamine, N-acetylglucosamine, N-acetylgalactosamine, N-acetylmannosamine, glucuronic acid, galacturonic acid, mannuronic acid, guluronic acid, iduronic acid, tagaturonic acid, frucuronic acid, galactosaminuronic acid, mannosaminuronic acid, glucosaminuronic acid, N-acetylglucosaminuronic acid, N-acetylgalactosaminuronic acid, N-acetylmannosaminuronic acid, maltose, lactose, sucrose, trehalose, gentiobiose, cellobiose, chitobiose, kojibiose, nigerose, sophorose, trehalulose, isomaltose, xylobiose, starch, cellulose, chitin, and dextran.

The carbohydrate moiety may comprise one or more monosaccharide moieties linked by a glycosidic bond. In some embodiments, the glycosidic bond comprises a 1->2 glycosidic bond, a 1->3 glycosidic bond, a 1->4 glycosidic bond, or a 1->6 glycosidic bond. In some embodiments, each glycosidic bonds may be present in the alpha or beta configuration. In an embodiment, the one or more monosaccharide moieties are linked directly by a glycosidic bond or are separated by a linker.

The term “carbohydrate” as used herein refers to compound comprising one or more monosaccharide moieties comprising at least 3 carbon atoms (e.g., arranged in a linear, branched, or cyclic structure) and an oxygen, nitrogen, or sulfur atom, or a fragment or variant of a monosaccharide moiety comprising at least 3 carbon atoms (e.g., arranged in a linear, branched, or cyclic structure) and an oxygen, nitrogen, or sulfur atom. Each monosaccharide moiety or fragment or variant thereof may be a tetrose, pentose, hexose, or heptose. Each monosaccharide moiety or fragment or variant thereof may exist as an aldose, ketose, sugar alcohol, and, where appropriate, in the L or D form. Exemplary monosaccharide moieties may be amino sugars, N-acetylamino sugars, imino sugars, deoxysugars, or sugar acids. Carbohydrates may comprise individual monosaccharide moieties, or may further comprise a disaccharide, oligosaccharide (e.g., a trisaccharide, tetrasaccharide, pentasaccharide, hexasaccharide, heptasaccharide, octasaccharide), a polysaccharide, or combinations thereof. Exemplary carbohydrates include ribose, arabinose, lyxose, xylose, deoxyribose, ribulose, xylulose, glucose, galactose, mannose, gulose, idose, talose, allose, altrose, psicose, fructose, sorbose, tagatose, rhamnose, pneumose, quinovose, fucose, mannuheptulose, sedoheptulose, galactosamine, mannosamine, glucosamine, N-acetylglucosamine, N-acetylgalactosamine, N-acetylmannosamine, glucuronic acid, galacturonic acid, mannuronic acid, guluronic acid, iduronic acid, tagaturonic acid, frucuronic acid, galactosaminuronic acid, mannosaminuronic acid, glucosaminuronic acid, N-acetylglucosaminuronic acid, N-acetylgalactosaminuronic acid, N-acetylmannosaminuronic acid, maltose, lactose, sucrose, trehalose, gentiobiose, cellobiose, chitobiose, kojibiose, nigerose, sophorose, trehalulose, isomaltose, xylobiose, starch, cellulose, chitin, and dextran.

The carbohydrate may comprise one or more monosaccharide moieties linked by a glycosidic bond. In some embodiments, the glycosidic bond comprises a 1->2 glycosidic bond, a 1->3 glycosidic bond, a 1->4 glycosidic bond, or a 1->6 glycosidic bond. In some embodiments, each glycosidic bonds may be present in the alpha or beta configuration. In an embodiment, the one or more monosaccharide moieties are linked directly by a glycosidic bond or are separated by a linker.

In some embodiments, the present disclosure features an inhibitor such as an siRNA complexed with a carbohydrate targeting moiety, wherein the carbohydrate targeting moiety includes an asialoglycoprotein receptor (ASGPR) binding moiety. The ASGPR is a C-type lectin primarily expressed on the sinusoidal surface of hepatocytes, and comprises a major (48 kDa, ASGPR-1) and a minor (40 kDa, ASGPR-2) subunit. The ASGPR is involved in the binding, internalization, and subsequent clearance of glycoproteins containing an N-terminal galactose (Gal) or N-terminal N-acetylgalactosamine (GaINAc) residues from circulation, such as antibodies. ASGPRs have also been shown to be involved in the clearance of low-density lipoprotein, fibronectin, and certain immune cells, and may be utilized by certain viruses for hepatocyte entry (see, e.g., Yang J., et al. J Viral Hepat. 13:158-165, 2006, and Guy, C S et al. Nat Rev Immunol. 8:874-887, 2011.

In some embodiments, the carbohydrate targeting moiety is mannose. For example, carbohydrate targeting moiety may be α-mannose or high-mannose. In some embodiments, the carbohydrate targeting moiety comprises a mannose 6-phosphate (M6P) or analog thereof. In an embodiment, the carbohydrate targeting moiety comprise a plurality of M6P moieties (e.g., M6Ps), e.g., at least 2, 3, 4, 5, 6, 7, 8, 9, 10, 11, 12, 13, 14, 15, 16, 17, 18, 19, 20, or more M6P moieties. In an embodiment, the carbohydrate targeting moiety comprises between 2 and 20 M6P moieties (e.g., 2, 3, 4, 5, 6, 7, 8, 9, 10, 11, 12, 13, 14, 15, 16, 17, 18, 19, or 20 M6P moieties). In an embodiment, the carbohydrate targeting moiety comprises between 2 and 10 M6P moieties (e.g., 2, 3, 4, 5, 6, 7, 8, 9, or 10 M6P moieties). In an embodiment, the carbohydrate targeting moiety comprises between 2 and 5 M6P moieties (e.g., 2, 3, 4, or 5 M6P moieties).

In some embodiments, the carbohydrate targeting moiety comprises a galactose (Gal), galactosamine (GaINH₂), or an N-acetylgalactosamine (GaINAc) moiety, for example, a Gal, GaINH₂, or GaINAc, or an analog thereof. In an embodiment, the carbohydrate targeting moiety comprises a GaINAc moiety (e.g., GaINAc). In an embodiment, the carbohydrate targeting moiety comprises a plurality of GaINAc moieties (e.g., GaINAcs), e.g., at least 2, 3, 4, 5, 6, 7, 8, 9, 10, 11, 12, 13, 14, 15, 16, 17, 18, 19, 20, or more GaINAc moieties (e.g., GaINAcs). In an embodiment, the carbohydrate targeting moiety comprises between 2 and 20 GaINAcs moieties (e.g., 2, 3, 4, 5, 6, 7, 8, 9, 10, 11, 12, 13, 14, 15, 16, 17, 18, 19, or 20 GaINAc moieties). In an embodiment, the carbohydrate targeting moiety comprises between 2 and 10 GaINAc moieties (e.g., 2, 3, 4, 5, 6, 7, 8, 9, or 10 GaINAc moieties). In an embodiment, the carbohydrate targeting moiety comprises between 2 and 5 GaINAc moieties (e.g., 2, 3, 4, or 5 GaINAc moieties). In an embodiment, the carbohydrate targeting moiety comprises 2 GaINAc moieties. In an embodiment, the carbohydrate targeting moiety comprises 3 GaINAc moieties. In an embodiment, the carbohydrate targeting moiety comprises 4 GaINAc moieties. In an embodiment, the carbohydrate targeting moieties comprises 5 GaINAc moieties. In some embodiments, the carbohydrate targeting moiety includes a mono-, di-, tri-, or tetra-GaINAc

In some embodiments, the GaINAc moiety comprises a structure of Formula (I):

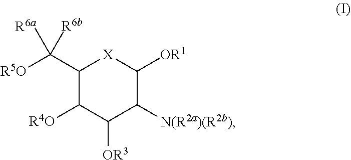

or a salt thereof, wherein X is O, N(R⁷), or S; each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; R^{2a} is hydrogen or alkyl; R^{2b} is —C(O)alkyl (e.g., C(O)CH3); each of R^{6a} and R^{6b} is hydrogen, alkyl, alkenyl, alkynyl, heteroalkyl, haloalkyl, halo, cyano, nitro, —OR^{A}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; and R^{A} is hydrogen, or alkyl, alkenyl, alkynyl, wherein the structure of Formula (I) may be connected to a linker at any position.

In some embodiments, X is O. In some embodiments, each of R¹, R³, R⁴, and R⁵ are independently hydrogen or alkyl (e.g., CH₃). In some embodiments, R^{2a} is hydrogen. In some embodiments, R^{2b} is C(O)CH₃. In some embodiments, each of R^{6a} and R^{6b} is hydrogen. In some embodiments, the GaINAc moiety is connected to a linker at R^{2a}. In some embodiments, the GaINAc moiety is connected to a linker at R^{2b}. In some embodiments, the GaINAc moiety is connected to a linker or at R³. In some embodiments, the GaINAc moiety is connected to a linker at R⁴. In some embodiments, the GaINAc moiety is connected to a linker at R⁵. In some embodiments, the GaINAc moiety is connected to a linker at R^{6a} or R^{6b}. In some embodiments, the GaINAc moiety is connected to a linker at a plurality of positions, e.g., at least two of R¹, R2a, R2b, R³, R⁴, R⁵, R^{6a}, and R^{6b}.

In some embodiments, the GaINAc moiety is comprises a structure of Formula (I-a)

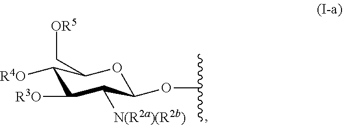

or a salt thereof, wherein R^{2a} is hydrogen or alkyl; R^{2b} is —C(O)alkyl (e.g., C(O)CH₃); each of R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; and R⁸ is hydrogen, halo, cyano, alkyl, alkenyl, alkynyl, heteroalkyl, haloalkyl, cycloalkyl, or heterocyclyl, wherein the “ [symbol] ” represents a bond in any configuration, and “ [symbol] ” represents an attachment point to a linker.

In some embodiments, each of R³, R⁴, and R⁵ are independently hydrogen or alkyl (e.g., CH₃). In some embodiments, R^{2a} is hydrogen. In some embodiments, R^{2b} is C(O)CH₃.

In some embodiments, the carbohydrate targeting moiety comprises a structure of Formula (II):

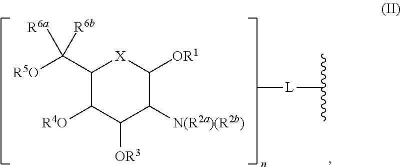

or a salt thereof, wherein each of R¹, R2a, R2b, R³, R⁴, R⁵, R^{6a}, and R^{6b} and subvariables thereof are as defined for Formula (I), L is a linker, and n is an integer between 1 and 100, wherein “ [symbol] ” represents an attachment point to a branching point, additional linker.

In some embodiments, X is O. In some embodiments, each of R¹, R³, R⁴, and R⁵ are independently hydrogen or alkyl (e.g., CH₃). In some embodiments, R^{2a} is hydrogen. In some embodiments, R^{2b} is C(O)CH₃. In some embodiments, each of R^{6a} and R^{6b} is hydrogen. In some embodiments, n is an integer between 1 and 50. In some embodiments, n is an integer between 1 and 25. In some embodiments, n is an integer between 1 and 10. In some embodiments, n is an integer between 1 and 5. In some embodiments, n is 1, 2, 3, 4, or 5. In some embodiments, n is 1.

In some embodiments, the carbohydrate targeting moiety comprises a structure of Formula (II-a):

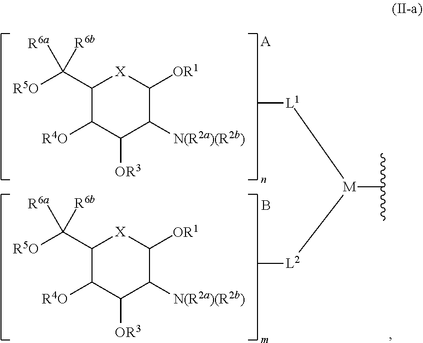

or a salt thereof, wherein each of R¹, R^{2a}, R^{2b}, R³, R⁴, R⁵, R^{6a}, and R^{6b} and subvariables thereof are as defined for Formula (I), each of L¹ and L² is independently a linker, each of m and n is independently an integer between 1 and 100, and M is a linker, wherein “ [symbol] ” represents an attachment point to a branching point, additional linker.

In some embodiments, X is O (e.g., X in each of A and B is O). In some embodiments, each of R¹, R³, R⁴, and R⁵ are independently hydrogen or alkyl (e.g., CH₃) (e.g., R¹, R³, R⁴, and R⁵ in each of A and B is independently hydrogen or alkyl). In some embodiments, R^{2a} is hydrogen (e.g., R^{2a} in each of A and B is hydrogen). In some embodiments, R^{2b} is C(O)CH₃ (e.g., R^{2b} in each of A and B is C(O)CH₃). In some embodiments, each of R^{6a} and R^{6b} is hydrogen (e.g., R^{6a} and R^{6b} in each of A and B is hydrogen). In some embodiments, each of m and n is independently an integer between 1 and 50. In some embodiments, each of m and n is independently an integer between 1 and 25. In some embodiments, each of m and n is independently an integer between 1 and 10. In some embodiments, each of m and n is independently an integer between 1 and 5. In some embodiments, each of m and n is independently 1, 2, 3, 4, or 5. In some embodiments, each of m and n is independently 1.

In an embodiment, each of L¹ and L² independently comprises an alkylene, alkenylene, alkynylene, heteroalkylene, or haloalkylene group. In an embodiment, each of L¹ and L² independently comprises an ester, amide, disulfide, ether, carbonate, aryl, heteroaryl, cycloalkyl, or heterocyclyl group. In an embodiment, each of L¹ and L² independently is cleavable or non-cleavable.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the ASGPR moiety comprises a structure of Formula (II-b):

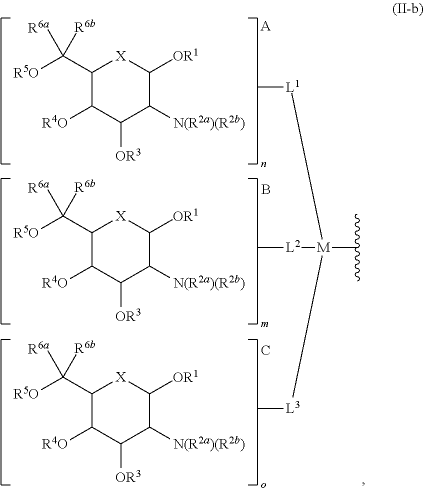

or a salt thereof, wherein each of R¹, R²³, R^{2b}, R³, R⁴, R⁵, R^{6a}, and R^{6b} and subvariables thereof are as defined for Formula (I), each of L¹, L², and L³ is independently a linker, each of m, n, and o is independently an integer between 1 and 100, and M is a linker, wherein “ [symbol] ” represents an attachment point to a branching point, additional linker.

In some embodiments, X is O (e.g., X in each of A, B, and C is O). In some embodiments, each of R¹, R³, R⁴, and R⁵ are independently hydrogen or alkyl (e.g., CH₃) (e.g., R¹, R³, R⁴, and R⁵ in each of A, B, and C is independently hydrogen or alkyl). In some embodiments, R^{2a} is hydrogen (e.g., R^{2a} in each of A, B, and C is hydrogen). In some embodiments, R^{2b} is C(O)CH₃ (e.g., R^{2b} in each of A, B, and C is C(O)CH₃). In some embodiments, each of R^{6a} and R^{6b} is hydrogen (e.g., R^{6a} and R^{6b} in each of A, B, and C is hydrogen). In some embodiments, each of m, n, and o is independently an integer between 1 and 50. In some embodiments, each of m, n, and o is independently an integer between 1 and 25. In some embodiments, each of m, n, and o is independently an integer between 1 and 10. In some embodiments, each of m, n, and o is independently an integer between 1 and 5. In some embodiments, each of m, n, and o is independently 1, 2, 3, 4, or 5. In some embodiments, each of m, n, and o is independently 1.

In an embodiment, each of L¹, L², and L³ independently comprises an alkylene, alkenylene, alkynylene, heteroalkylene, or haloalkylene group. In an embodiment, each of L¹, L², and L³ independently comprises an ester, amide, disulfide, ether, carbonate, aryl, heteroaryl, cycloalkyl, or heterocyclyl group. In an embodiment, each of L¹, L², and L³ independently is cleavable or non-cleavable.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the carbohydrate targeting moiety comprises a structure of Formula (II-c):

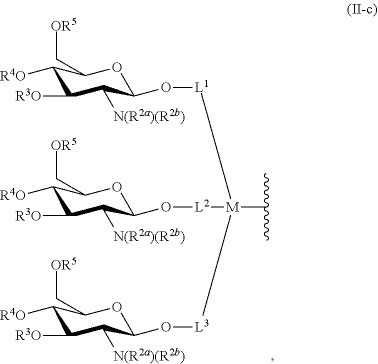

or a salt thereof, wherein each of R^{2a}, R^{2b}, R³, R⁴, R⁵, and subvariables thereof are as defined for Formula (I), each of L¹, L², and L³ is independently a linker, and M is a linker, wherein “ [symbol] ” represents an attachment point to a branching point, additional linker.

In some embodiments, each of R³, R⁴, and R⁵ are independently hydrogen or alkyl (e.g., CH₃). In some embodiments, R^{2a} is hydrogen. In some embodiments, R^{2b} is C(O)CH₃.

In an embodiment, each of L¹, L², and L³ independently comprises an alkylene, alkenylene, alkynylene, heteroalkylene, or haloalkylene group. In an embodiment, each of L¹, L², and L³ independently comprises an ester, amide, disulfide, ether, carbonate, aryl, heteroaryl, cycloalkyl, or heterocyclyl group. In an embodiment, each of L¹, L², and L³ independently is cleavable or non-cleavable.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the carbohydrate targeting moiety comprises a compound selected from:

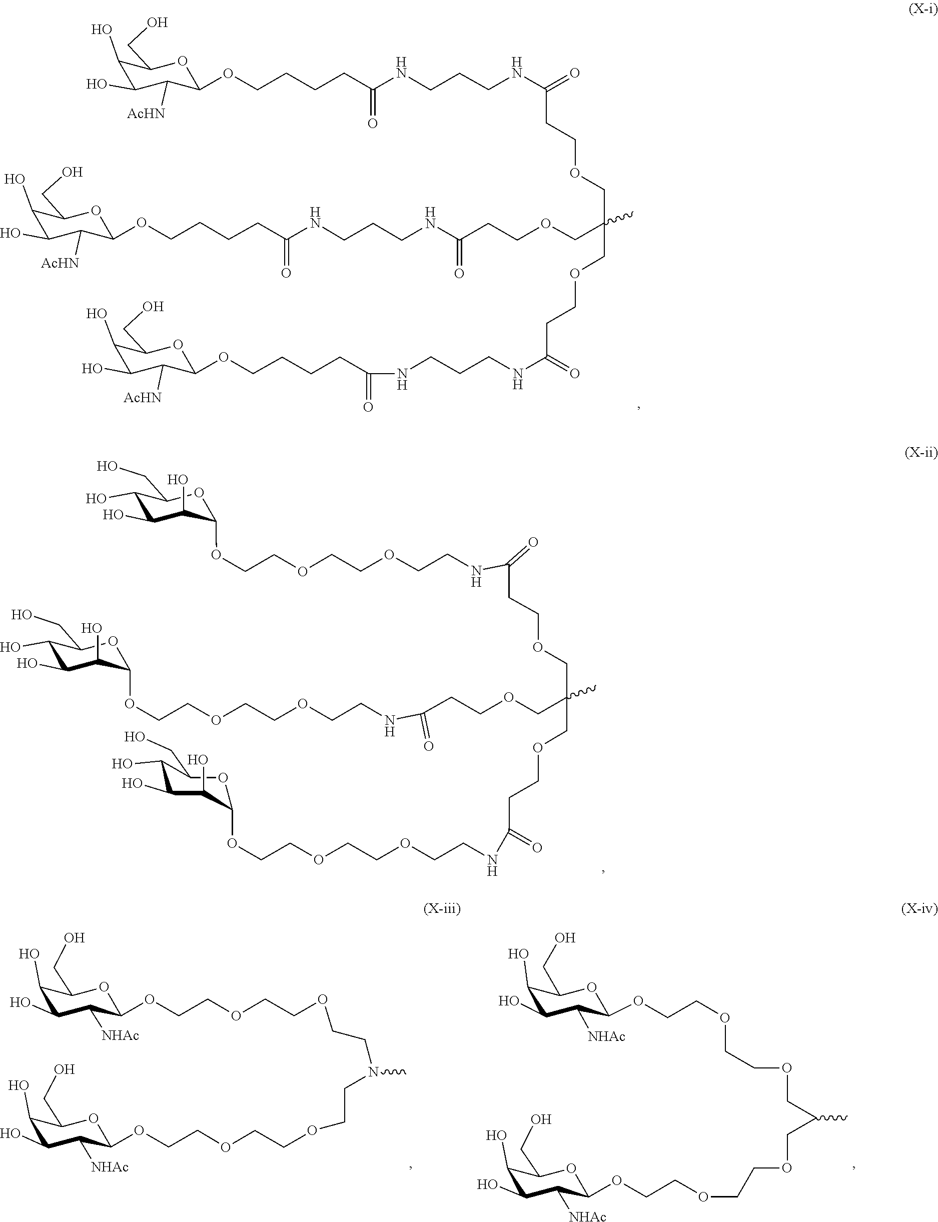

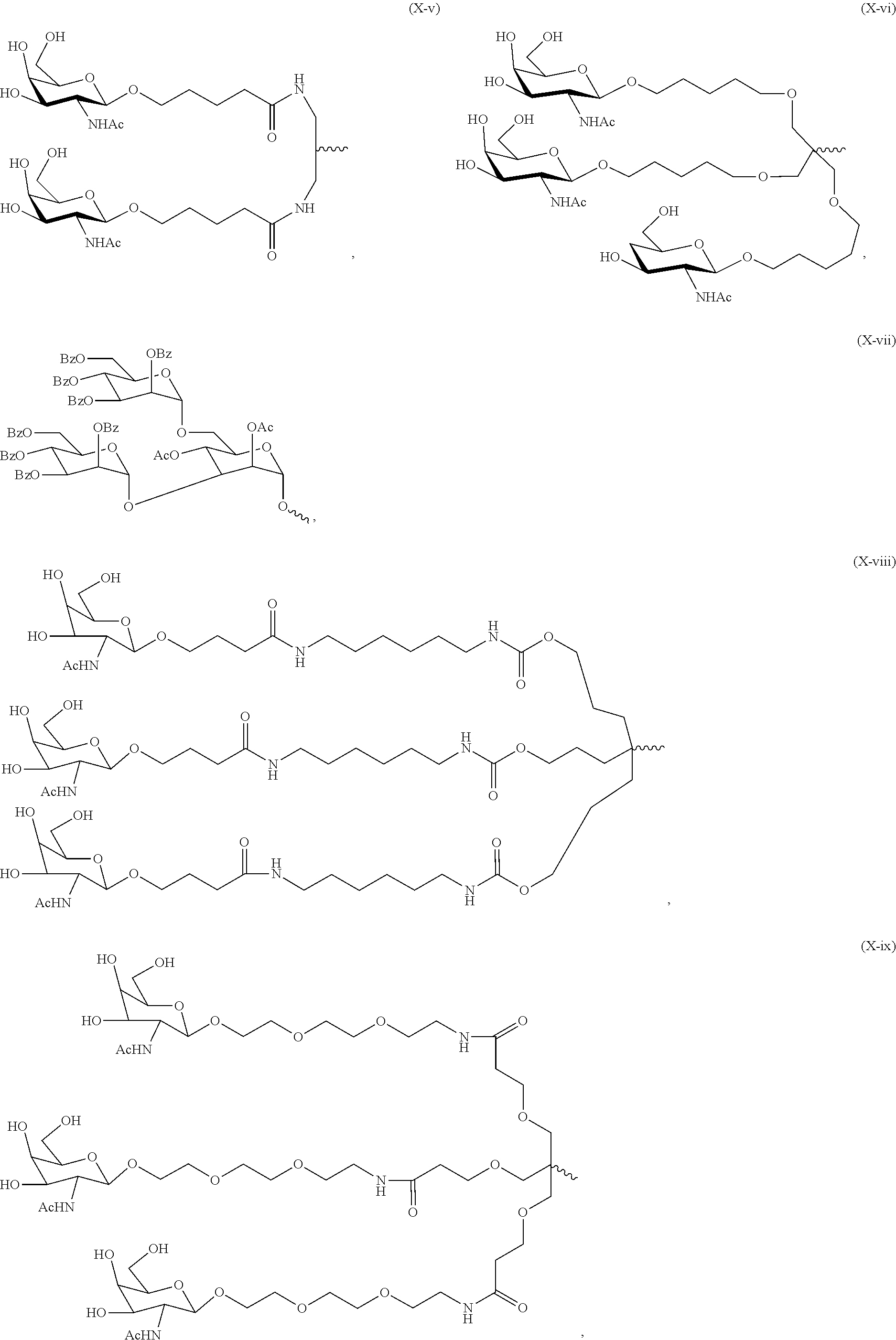

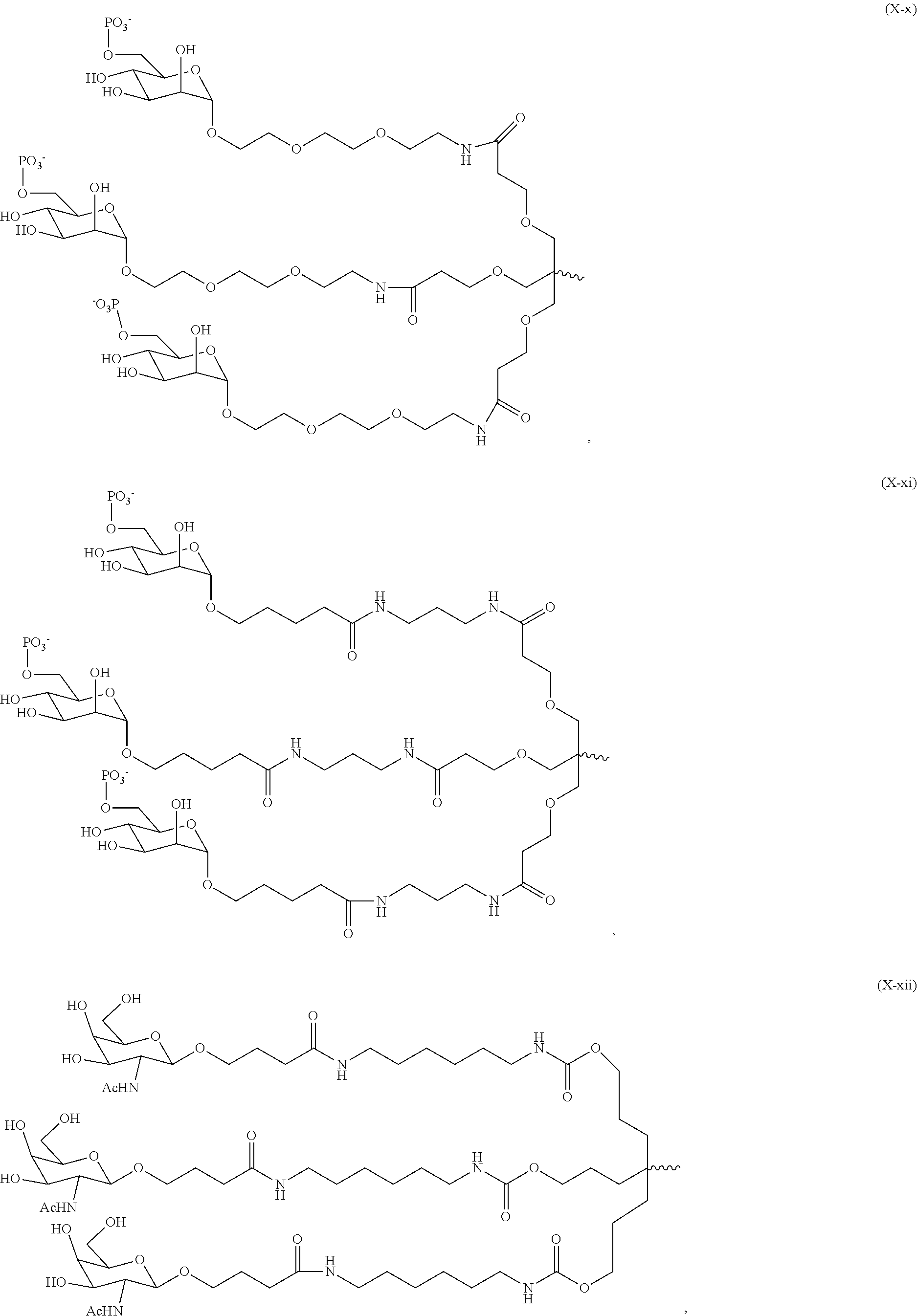

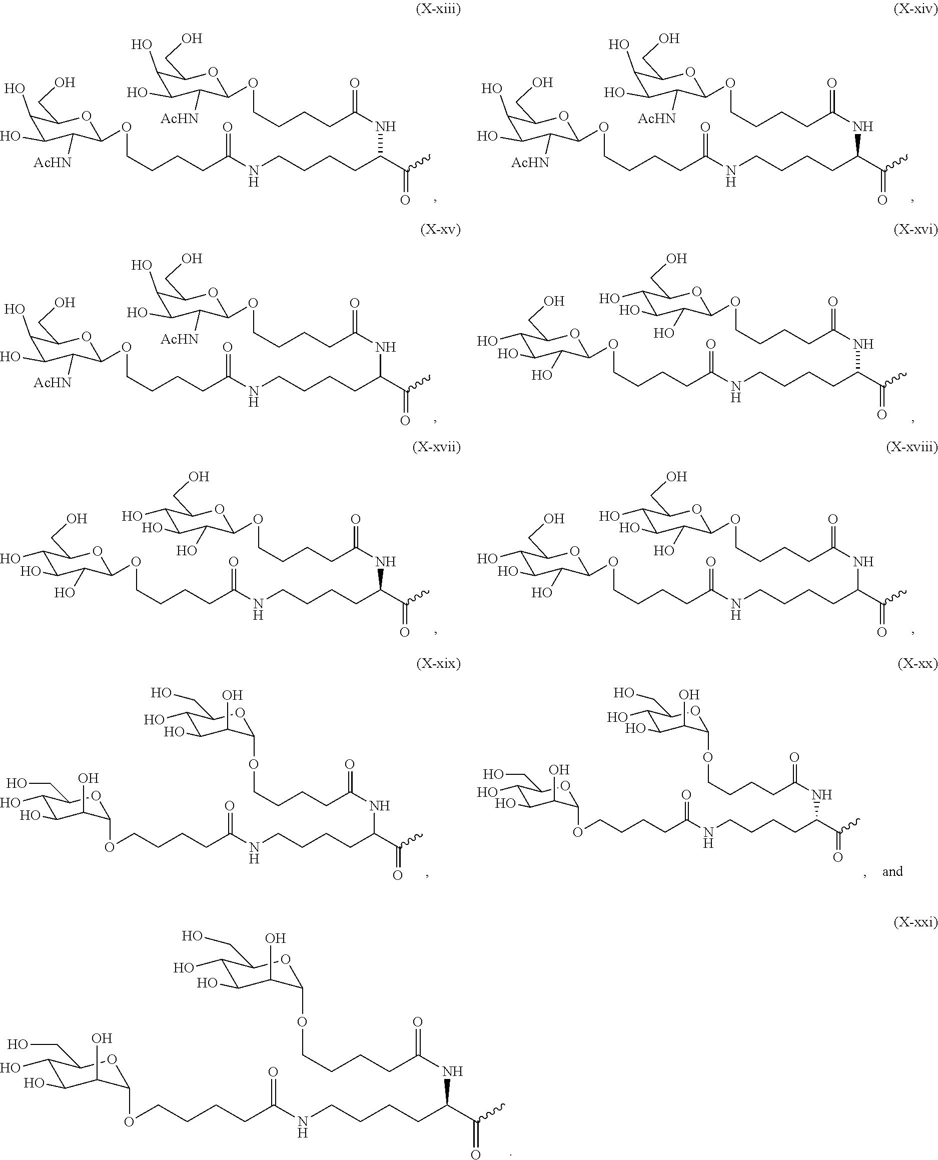

In some embodiments, the carbohydrate targeting moiety comprises a linker comprising a cyclic moiety, such as a pyrroline ring. In an embodiment, the carbohydrate targeting moiety comprises a structure of Formula (CII):

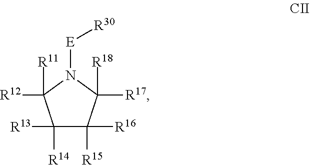

or a salt thereof, wherein E is absent or C(O), C(O)O, C(O)NH, C(S), C(S)NH, SO, SO₂, or SO₂NH; R¹¹, R¹², R¹³, R¹⁴, R¹⁵, R¹⁶, R¹⁷, and R¹⁸ are each independently for each occurrence H, —CH₂OR^{a}, or OR^{b}; R^{a} and R^{b} are each independently for each occurrence hydrogen, a hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group, a phosphoramidite, a solid support, —P(Z¹)(Z²)—O-nucleoside, —P(Z¹)(Z²)—O-oligonucleotide, —P(Z¹)(0-linker-R^{L})—O-nucleoside, or —P(Z¹)(O-linker-R^{L})-O-oligonucleotide; R³⁰ is independently for each occurrence-linker-R^{L} or R³¹; R^{L} is hydrogen or a ligand; R³¹ is —C(O)CH(N(R³²)₂)(CH₂)ₕN(R³²)₂; R³² is independently for each occurrence H, —R^{L}, -linker-R^{L} or R³¹; Z¹ is independently for each occurrence 0 or S; Z² is independently for each occurrence O, S, N(alkyl) or optionally substituted alkyl; and h is independently for each occurrence 1-20.

In some embodiments, the compound of Formula (CII) is selected from:

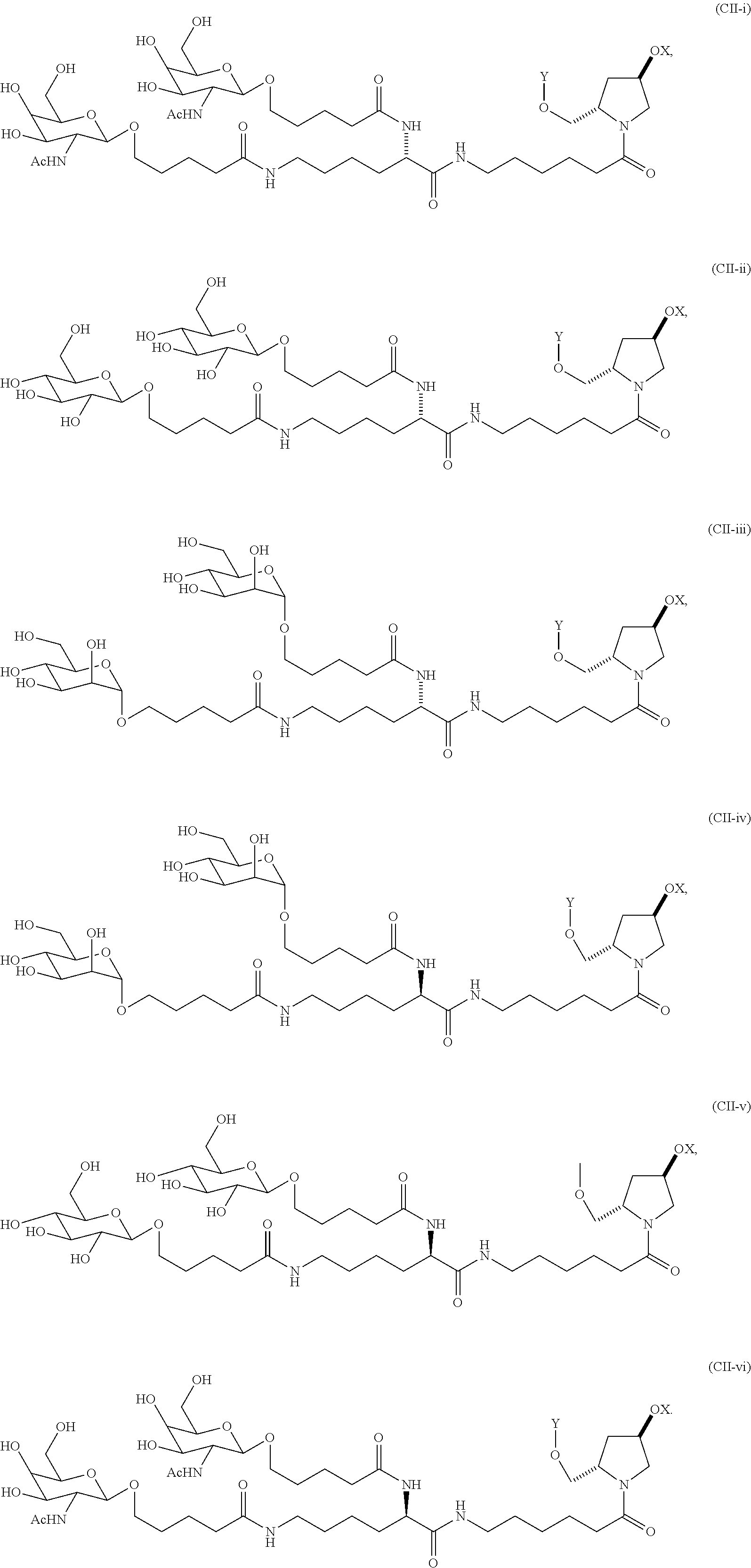

In some embodiments, the carbohydrate targeting moiety is a compound or substructure disclosed in U.S. Pat. No. 8,106,022, which is incorporated herein by reference in its entirety.

In other embodiments, the carbohydrate targeting moiety is selected from:

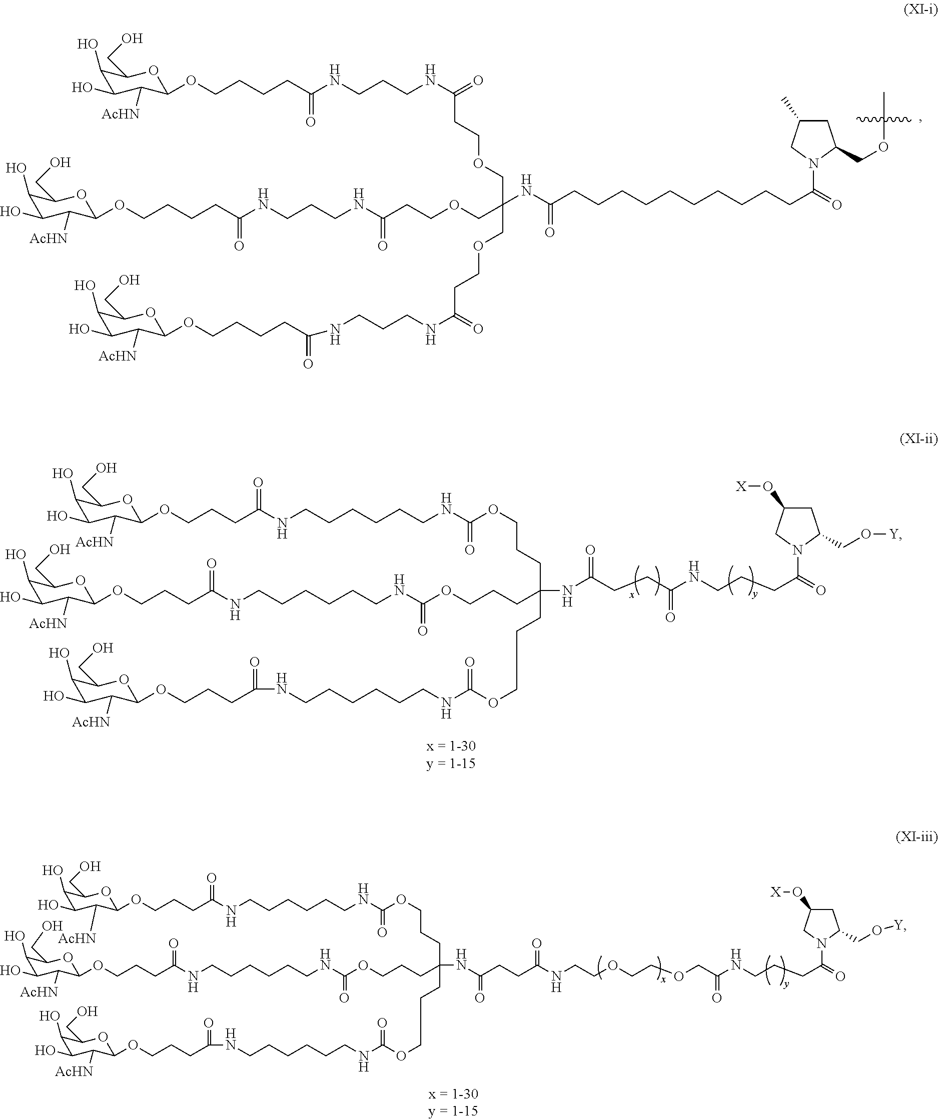

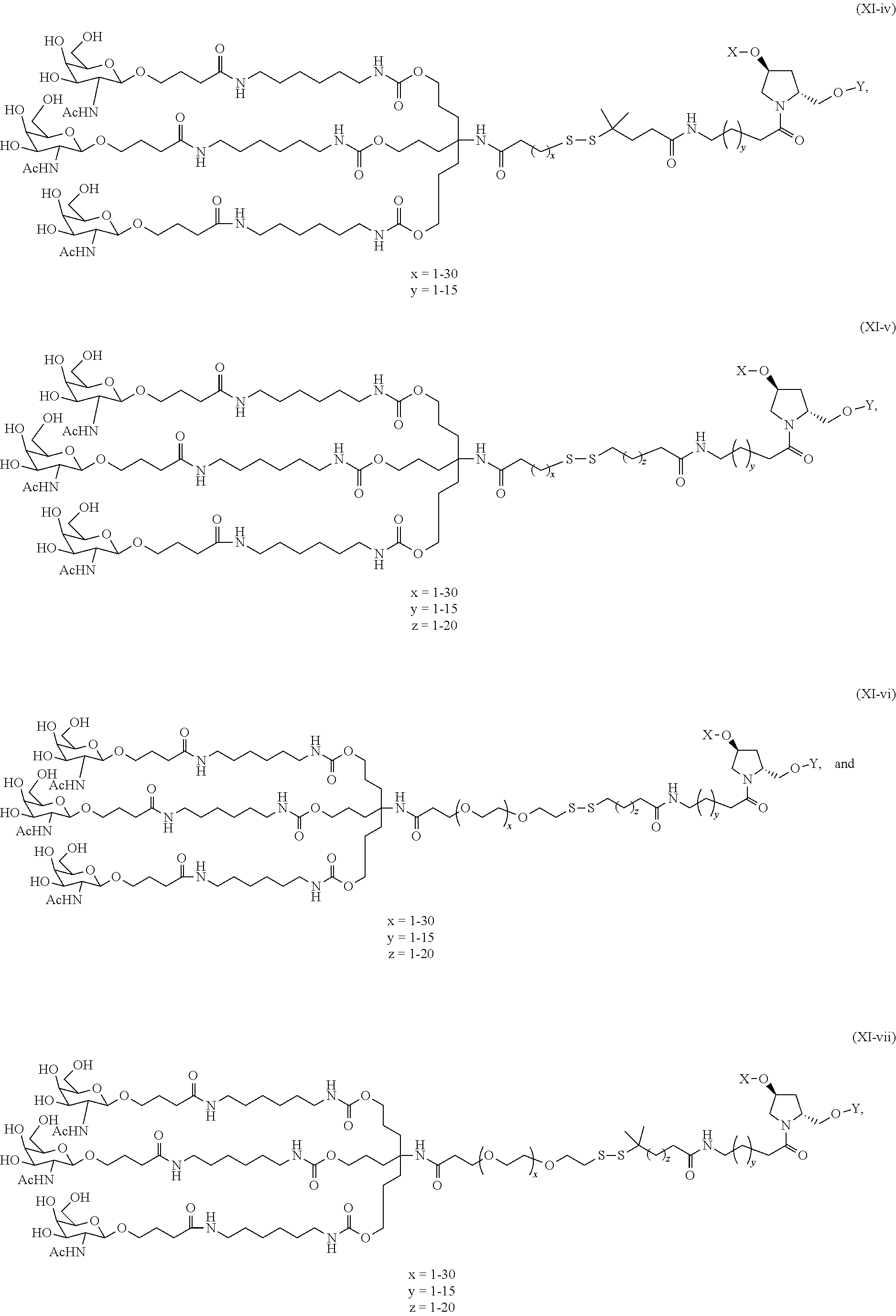

wherein one of X or Y is a branching point or a linker, and the other of X and Y is hydrogen.

In an embodiment, the ASGPR moiety comprises a structure of Formula (XII-a):

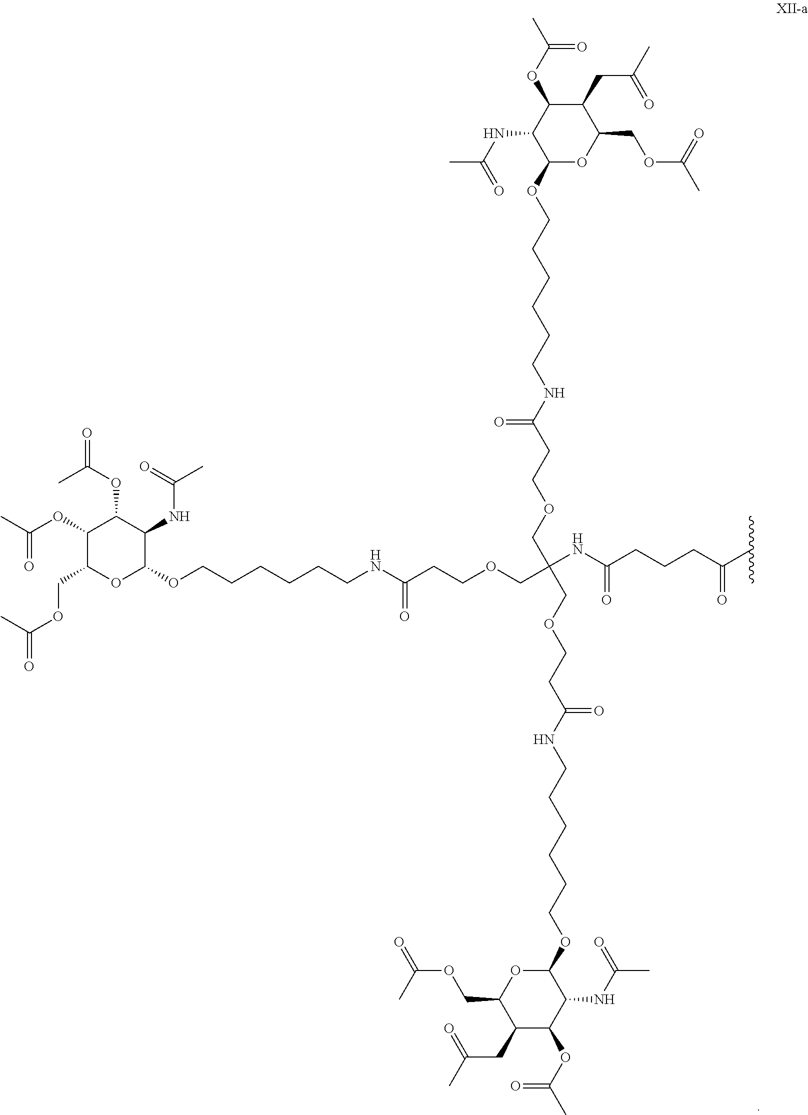 [SMILES: *C(=O)CCCC(=O)NC(COCCC(=O)NCCCCCCO[C@@H]1O[C@H](COC(C)=O)[C@H](OC(C)=O)[C@H](OC(C)=O)[C@H]1NC(C)=O)(COCCC(=O)NCCCCCCO[C@@H]1O[C@H](COC(C)=O)[C@H](CC(C)=O)[C@H](OC(C)=O)[C@H]1NC(C)=O)COCCC(=O)NCCCCCCO[C@@H]1O[C@H](COC(C)=O)[C@H](CC(C)=O)[C@H](OC(C)=O)[C@H]1NC(C)=O]

In an embodiment, the carbohydrate targeting moiety is a compound or substructure disclosed in Nucleic Acids (2016) 5:e317 or WO2015/042447, each of which is incorporated herein by reference in its entirety.

In some embodiments, the carbohydrate targeting moiety comprises a structure of Formula (V-a):

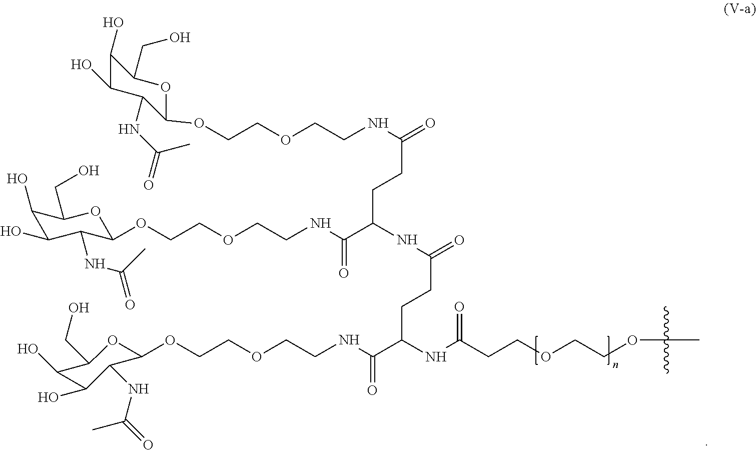

wherein n is an integer from 1 to 20. In some embodiments, the compound of Formula (V-a) is selected from:

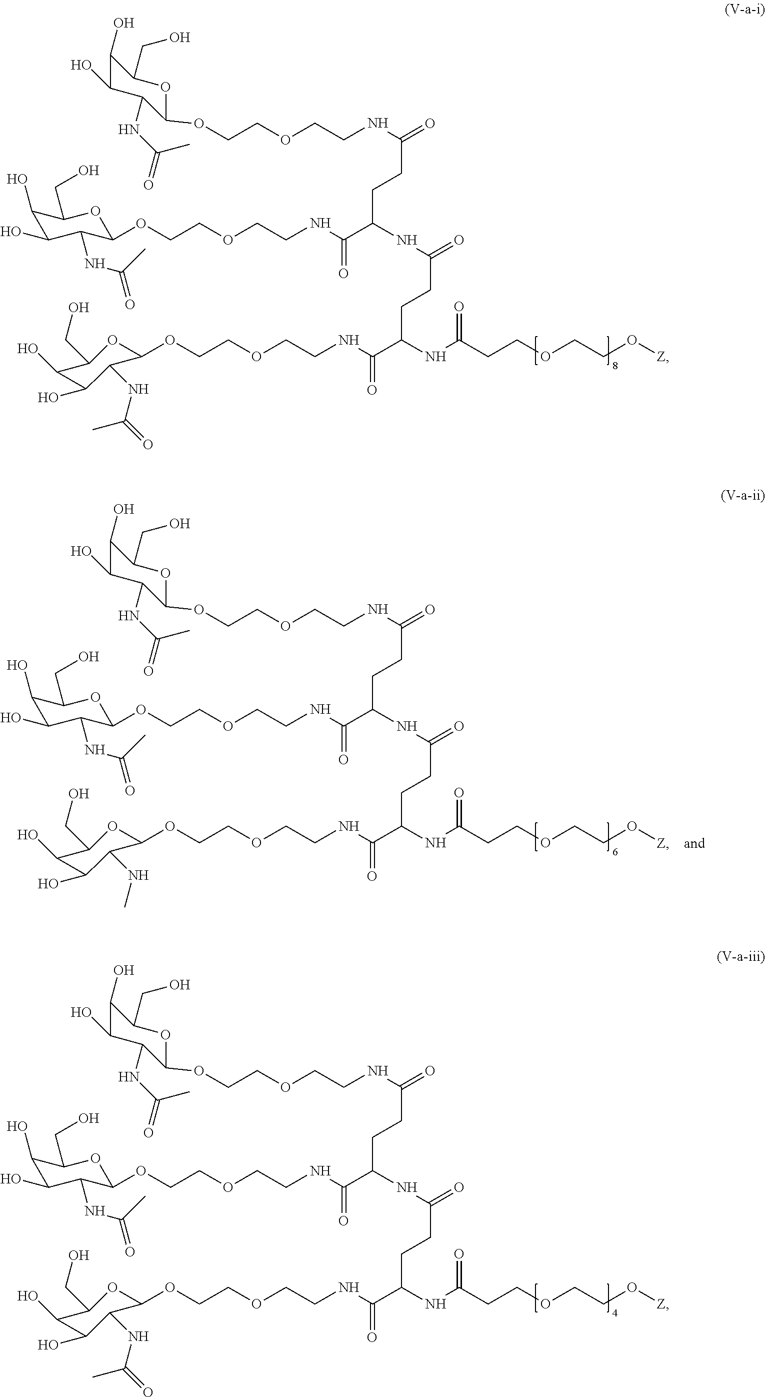

wherein Z is an oligomeric compound, e.g., a linker.

In another embodiment, the carbohydrate binding moiety comprises a structure of Formula (V-b):

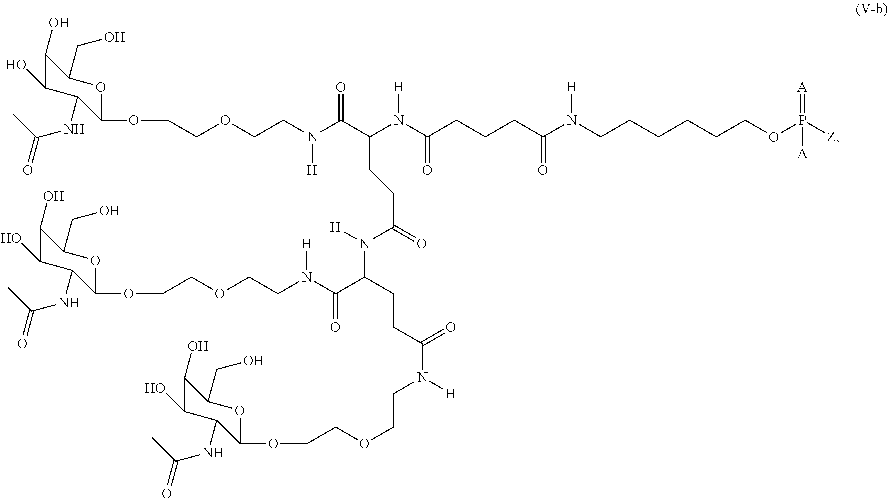

wherein A is O or S, A′ is O, S, or NH, and Z is an oligomeric compound, e.g., a linker.

In some embodiments, the carbohydrate targeting moiety comprises

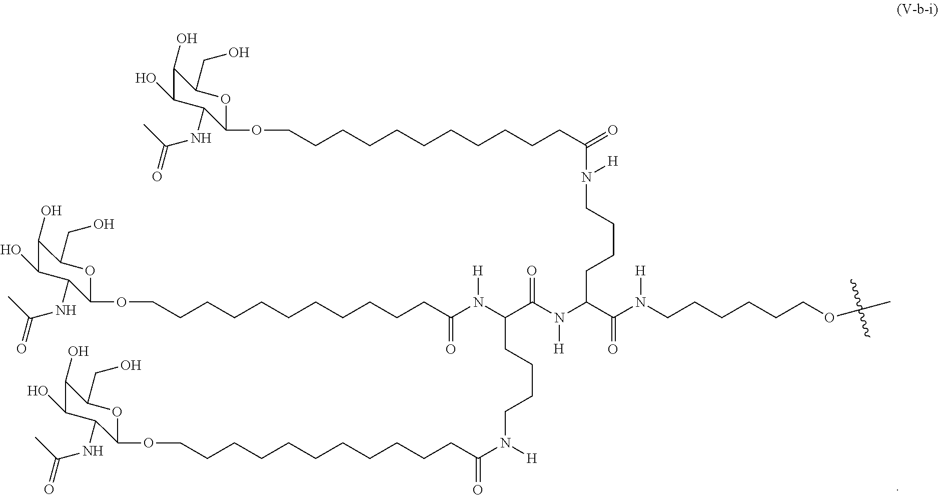 [SMILES: *OCCCCCCNC(=O)C(CCCCNC(=O)CCCCCCCCCCCOC1OC(CO)C(O)C(O)C1NC(C)=O)NC(=O)C(CCCCNC(=O)CCCCCCCCCCCOC1OC(CO)C(O)C(O)C1NC(C)=O)NC(=O)CCCCCCCCCCCOC1OC(CO)C(O)C(O)C1NC(C)=O]

In some embodiments, the carbohydrate targeting moiety is selected from:

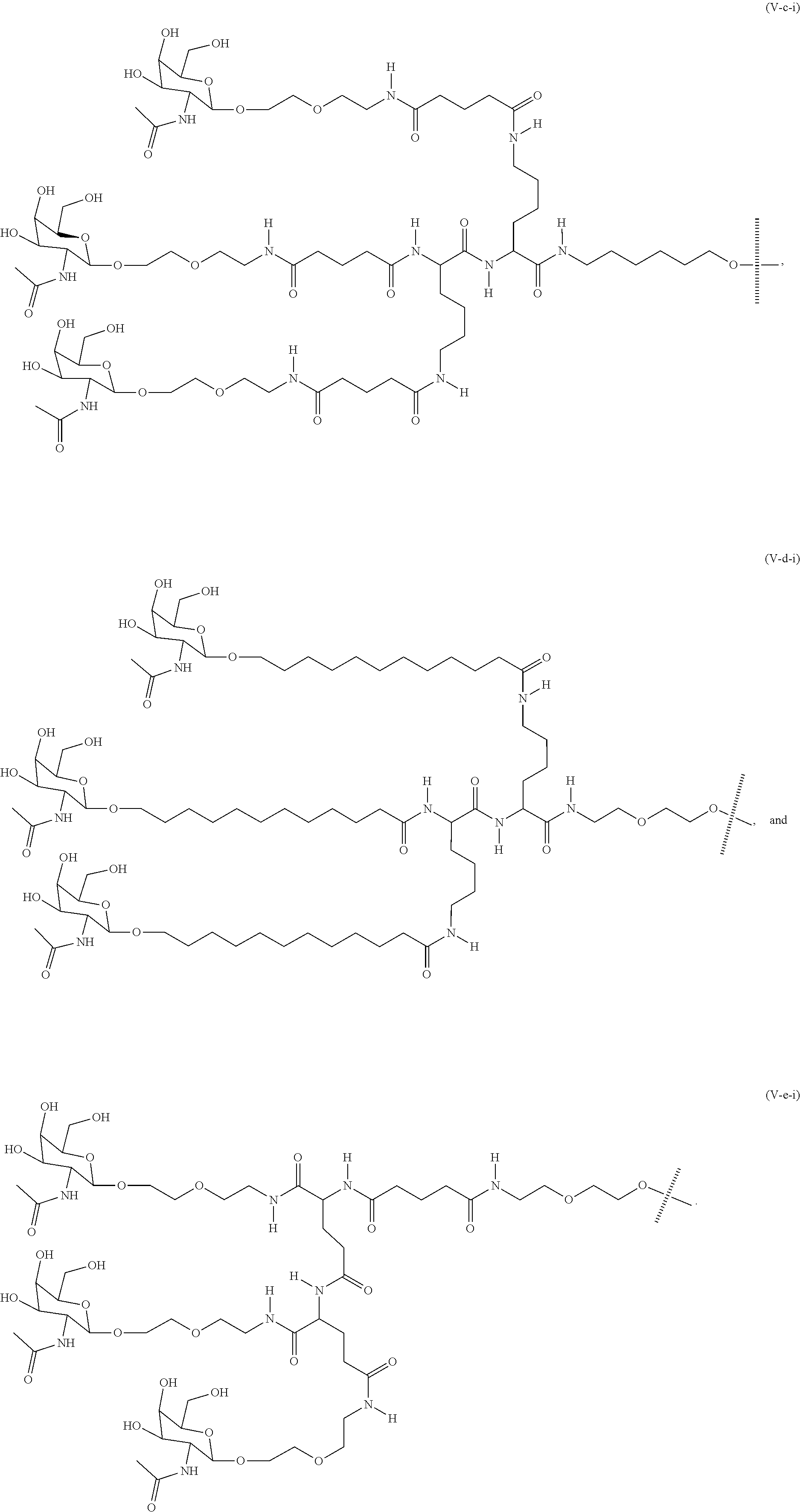

In an embodiment, the carbohydrate targeting moiety is a compound or substructure disclosed in WO 2017/156012, which is incorporated herein by reference in its entirety.

In some embodiments, a hydroxyl group within a carbohydrate targeting moiety is protected, for example, with an acetyl or acetonide moiety. In some embodiments, a hydroxyl group within a carbohydrate targeting moiety is protected with an acetyl group. In some embodiments, a hydroxyl group within an ASGPR moiety is protected with acetonide group. For example, 1, 2, 3, 4, 5, 6, 7, 8, 9, 10, 11, 12, or more hydroxyl groups within a carbohydrate targeting moiety may be protected, e.g., with an acetyl group or an acetonide group. In some embodiments, all of the hydroxyl groups within a carbohydrate targeting moiety are protected.

In some embodiments, the carbohydrate targeting moiety comprises an additional active agent, such as a ligand (e.g., a steroid). The ligand may be covalently or non-covalently associated with the carbohydrate targeting moiety. For example, the ligand may be covalently bound to a carbohydrate, linker, or a branching point within the carbohydrate targeting moiety. In some embodiments, the carbohydrate targeting moiety comprises

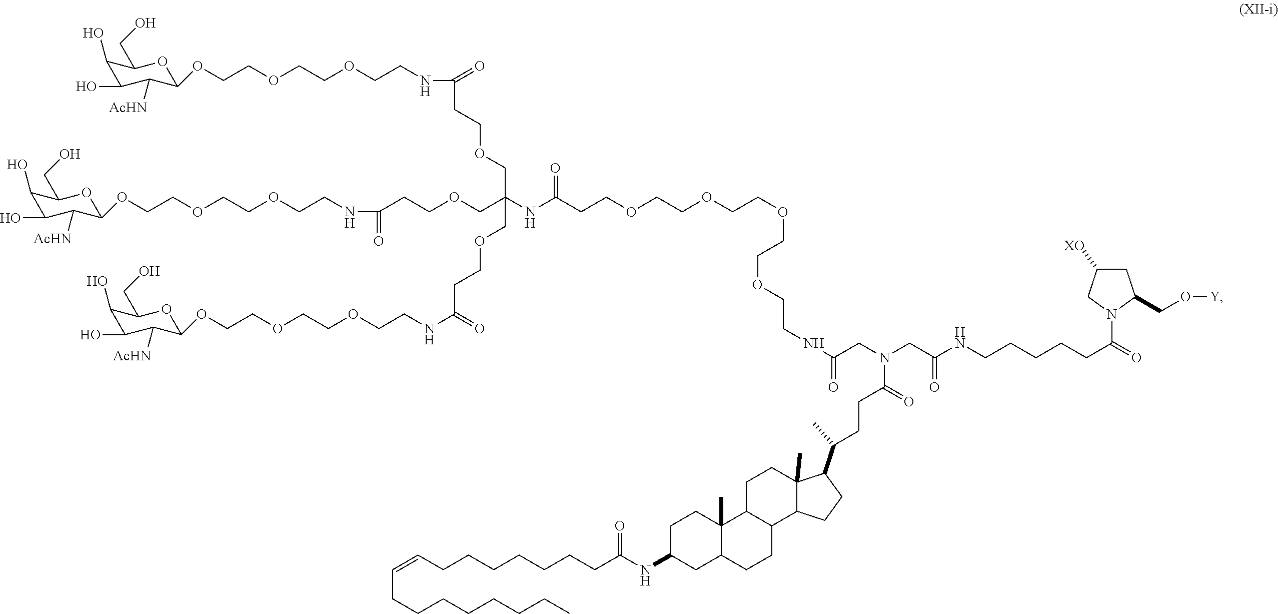

wherein one of X or Y is a branching point or linker, and the other of X and Y is hydrogen.

Additional exemplary carbohydrate targeting moieties are described in further detail in U.S. Pat. Nos. 8,828,956; 9,867,882; 10,450,568; and 10,808,246, each of which is incorporated herein by reference in its entirety.

Any of the above-described carbohydrate targeting moieties may also be biotinylated to facilitate binding to a biotin-binding protein (e.g., avidin, streptavidin, NeutrAvidin).

### Methods of Treatment

Treatment with an Agent that Inhibits a Target

A subject that has been identified as having or at risk of developing NAFLD (e.g., NAFL or NASH) can be treated with an agent described herein (e.g., an agent listed in Table 2, Table 3, or Table 4). In some embodiments, the subject is diagnosed using clinical diagnostic methods known in the art, such as a biopsy of one or more tissues (e.g., from the liver) to detect steatosis, inflammation, and/or fibrosis, magnetic resonance imaging (MRI) to detect or measure steatosis and/or liver enlargement, magnetic resonance elastography (MRE) to detect or measure fibrosis, an ultrasound to detect steatosis and/or fibrosis, among other biomarkers and diagnostic methods known in the art. In addition to the methods of treatment described herein, the subject may be treated with a standard of care therapy, such as proper diet and exercise, bariatric surgery, and/or may be treated with drugs to induce weight loss. A subject may also be identified as at risk of developing NAFLD or at risk of progressing from NAFL to NASH based on a family history of NAFLD or other associated risk factors described herein (e.g., obesity). Treatment of a subject developing or at risk of developing NAFLD may reduce the likelihood of developing NAFL or NASH, inhibit the onset of NAFL or NASH, and/or delay the onset of NAFL or NASH. In some embodiments, the methods of treating a subject having NAFLD (e.g., NAFL or NASH) may further be used to reduce, slow, inhibit, and/or reverse the progression of steatosis, inflammation, fibrosis, and/or hepatocellular ballooning.

A subject that has been diagnosed as having NAFL or NASH (e.g., using standard diagnostic approaches) is treated with an agent described herein (e.g., an agent listed in Table 2, Table 3, or Table 4). In some embodiments, treatment is used to reduce, slow, and/or inhibit the progression of NAFLD (e.g., NAFL or NASH), and/or reverse NAFLD (e.g., NAFL or NASH) in a subject diagnosed as having the disease. In some embodiments, the methods may further be used to reduce, slow, inhibit, and/or reverse the progression of steatosis in a subject diagnosed with NAFL or NASH. In some embodiments, the methods may be used to stabilize a condition of a subject (e.g., stabilize the subject's NAFL or NASH such that it does not progress or get worse). In other embodiments, the methods may be used to alleviate, ameliorate, reduce, or reverse clinical manifestations of the disease (e.g., alleviate, ameliorate, reduce, or reverse the progression of liver inflammation, hepatic steatosis, and/or liver fibrosis).

The method may include delivering an agent of the disclosure, or a pharmaceutical composition containing the same, to the liver, muscle, blood, or any affected tissues of the subject (e.g., a human) by any appropriate route of administration (e.g., subcutaneous, intravenous, intramuscular, etc.). The method may include delivering an agent of the disclosure, or a pharmaceutical composition containing the same, to hepatocytes by any appropriate route of administration. Exemplary routes of administration are oral administration, intravenous injection, subcutaneous injection, or intramuscular injection. The agent can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, week, month or year, and for as long as necessary. Subjects may be adult or pediatric humans, with or without a comorbid condition.

### Combination Therapies

The method may include administration of an agent (e.g., an agent listed in Table 2, Table 3, or Table 4) in combination with one or more additional therapies (e.g., 1, 2, or 3 additional agents). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each agent can be effected by any appropriate route including, but not limited to, oral routes, intravenous routes, intramuscular routes, local routes, and direct absorption through mucous membrane tissues. The agents can be administered by the same route or by different routes. For example, a first agent of the combination may be administered by intravenous injection while a second agent of the combination can be administered locally. The first agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second agent.

For use in treating NAFL or NASH (e.g., in a subject diagnosed with NAFL or NASH or is at risk of developing either of these conditions), the second inhibitor may be one or more agents listed in Table 2, Table 3, or Table 4. In some embodiments, the two or more inhibitors are inhibitors of different targets listed in Table 1. In some embodiments, the same target listed in Table 1 is inhibited by two or more therapeutic platforms listed in Table 2. In other embodiments, the same target listed in Table 1 is inhibited by two or more inhibitory nucleic acid molecules such as, e.g., two or more siRNAs listed in Table 3 or Table 4. One or more inhibitors against one or more targets listed in Table 1 may be used in combination with other standard of care therapies for use in treating NAFL or NASH in a subject in need thereof.

### Evaluating Efficacy

Determining the efficacy of a method of treatment directed to a subject having or at risk of developing NAFLD (e.g., NAFL or NASH) may require evaluating the subject's response to the treatment. The subject's response may be evaluated in an in-patient treatment setting or an out-patient treatment setting. Evaluation of the subject's response may occur one or more times following the administration of one or more inhibitors (e.g., one or more inhibitors of a target listed in Table 1). Evaluation of the subject's response may occur continuously, either sporadically or at designated time points following administration of the one or more inhibitor. Evaluation of the subject's response may occur, for example, 1-10 days after administration of the one or more inhibitor (e.g., 7-10 days, 6-8 days, 5-7 days, 3-5 days, 2-4 days, 1-3 days, or within 1 day following administration of the one or more inhibitor; e.g., 10 days, 9 days, 8 days, 7 days, 6 days, 5 days, 4 days, 3 days, 2 days, 1 day, or less than a day following administration of the one or more inhibitor). Evaluation of the subject's response may occur 1-12 weeks or more after administration of the one or more inhibitor (e.g., 10-12 weeks, 8-12 weeks, 6-12 weeks, 8-10 weeks, 6-10 weeks, 4-10 weeks, 6-8 weeks, 4-8 weeks, 2-8 weeks, 4-6 weeks, 2-6 weeks, 3-6 weeks, 2-4 weeks, 1-3 weeks, or 1-2 weeks following administration of the one or more inhibitor; e.g., later than 12 weeks, 12 weeks, 11 weeks, 10 weeks, 9 weeks, 8 weeks, 7 weeks, 6 weeks, 5 weeks, 4 weeks, 3 weeks, 2 weeks, or 1 week following administration of the one or more inhibitor). Evaluation of the subject's response may occur 1-12 months after administration of the one or more inhibitor (e.g., 10-12 months, 8-10 months, 6-8 months, 4-6 months, 3-5 months, 2-4 months, or 1-3 months following administration of the one or more inhibitor; e.g., 12 months, 11 months, 10 months, 9 months, 8 months, 7 months, 6 months, 5 months, 4 months, 3 months, 2 months, or 1 month following administration of the one or more inhibitor). Evaluation of the subject's response may occur 1-5 years or longer after administration of the one or more inhibitor (e.g., 4-5 years, 3-5 years, 2-3 years, or 1-3 years following administration of the one or more inhibitor; e.g., later than 5 years, 5 years, 4 years, 3 years, 2 years, or 1 year following administration of the one or more inhibitor).

The efficacy of the method of treatment may be evaluated by comparing one or more metrics of a subject that is administered the treatment compared to a relevant control or reference. In some embodiments, the efficacy of the method of treatment is evaluated by comparing one or more metrics of a subject undergoing treatment to one or more metrics of the same subject prior to treatment. In some embodiments, the efficacy of the method of treatment is evaluated by comparing one or more metrics of a subject undergoing treatment to one or more metrics of a different subject who has or is at risk of developing NAFLD and has not undergone treatment. In further embodiments, the efficacy of the method of treatment is evaluated by comparing one or more metrics of a subject undergoing treatment to one or more metrics of a healthy subject (e.g., one who does not have or is not presently at risk of developing NAFLD).

In some embodiments, the efficacy of the method of treatment directed to a subject having or at risk of developing NAFLD is evaluated by histopathological analysis of steatosis, inflammation, and/or fibrosis in the liver tissue of the subject by one or more methodologies known in the art. In some embodiments, a histopathological analysis of the liver tissue or other tissues is conducted by one or more biopsies over the course of treatment to evaluate steatosis, inflammation, and/or fibrosis of the liver. In some embodiments, treatment efficacy is evaluated by one or more magnetic resonance images (MRI) to detect or measure steatosis over the course of treatment. In some embodiments, treatment efficacy is evaluated one or more magnetic resonance elastographs (MRE) to detect or measure fibrosis over the course of treatment. In further embodiments, treatment efficacy is evaluated by one or more ultrasounds (e.g., FIBROSCAN®) to detect or measure steatosis and/or fibrosis over the course of treatment.

In some embodiments, the level of liver steatosis in a subject having or at risk of developing NAFLD is reduced following the administration of one or more inhibitors described herein such that the level of liver steatosis is reduced by about 5%, 10%, 20%, 30%, 40%, 50%, 60%, 70%, 80%, 90% or more than 90% compared to levels of liver steatosis in the subject prior to treatment. In some embodiments, the total amount of lipids or triglycerides in the liver is reduced to levels comparable to a healthy subject (e.g., less than 5% triglyceride content). In some embodiments the level of liver inflammation in a subject having or at risk of developing NAFLD is reduced following the administration of one or more inhibitors described herein such that the level of liver inflammation is reduced by about 5%, 10%, 20%, 30%, 40%, 50%, 60%, 70%, 80%, 90% or more than 90% compared to levels of liver inflammation in the subject prior to treatment. In further embodiments, the level of liver fibrosis in a subject having or at risk of developing NAFLD is reduced following the administration of one or more inhibitors described herein such that the level of liver fibrosis is reduced by about 5%, 10%, 20%, 30%, 40%, 50%, 60%, 70%, 80%, 90% or more than 90% compared to levels of liver fibrosis in the subject prior to treatment.

In some embodiments, the efficacy of the method of treatment directed to a subject having or at risk of developing NAFLD is evaluated by measuring the expression or activity of one or more targets listed in Table 1 over the course of treatment. In some embodiments, the expression or activity of the one or more targets listed in Table 1 are reduced by about 5%, 10%, 20%, 30%, 40%, 50%, 60%, 70%, 80%, 90%, or more following the administration to the subject of one or more inhibitors against a target listed in Table 1 (e.g., an inhibitor listed in Table 2, Table 3, or Table 4). In some embodiments, the expression or activity of one or more targets listed in Table 1 is evaluated by taking one or more biological samples from the subject (e.g., a blood sample or component thereof or a liver biopsy) and comparing the results from earlier measurements from the subject and/or results from the subject that were measured prior to administration of the one or more inhibitor. In some embodiments, the expression or activity of the one or more targets listed in Table 1 is detected by biological or analytical methods known in the art (e.g., mass spectrometry, Western blot analysis, ELISA, RT-PCR, flow cytometry, immunofluorescence, colorimetry assays, an array using targeting antibodies or hybridizing nucleotides, among other methods known in the art).

Additional biomarkers described herein may be used to evaluate the efficacy of treatment administered to a subject in need thereof. Efficacy of treatment may be evaluated by measuring blood or serum levels of one or more biomarkers such as, e.g., retinol binding protein 4 (RBP4), adiponection (ADIPOQ), leptin (LEP), resistin (RETN), or ghrelin (GHRL); liver transaminase level such as the AST/ALT ratio; cholesterol level, such as low-density lipoprotein (LDL)-cholesterol or a reduced level of high-density lipoprotein (HDL)-cholesterol; triglyceride level; uric acid levels; among other biomarkers described herein or known in the art.

Depending on the efficacy of the treatment administered to the subject having or at risk of developing NAFLD, the treatment regimen may change over the course of treatment. The treatment regimen and efficacy thereof may be determined by a practitioner skilled in the art (e.g., a physician, clinician, or medical specialist). In some embodiments, the dose of the one or more inhibitor of a target listed in Table 1 may be adjusted (e.g., decreased or increased) over the course of treatment. In some embodiments, the dose of the one or more inhibitors may be increased by about 10%, 25%, 50%, 75%, 100%, 125%, 150%, 175%, 200%, or more over the course of treatment. In some embodiments, the dose of the one or more inhibitors may be decreased by about 10%, 25%, 50%, 75%, or more than 75% over the course of treatment. In some embodiments, the frequency of administration of the one or more inhibitors may increase or decrease over the course of treatment. In further embodiments, the duration of treatment may increase or decrease. In some embodiments, a different combination of inhibitors may be administered such that a new combination of different types of inhibitors listed in Table 2, Table 3, or Table 4 are administered to the subject in need thereof over the course of treatment. In other embodiments, different combinations of the same agent of inhibitors (e.g., two or more siRNAs listed in Table 3 or Table 4) may be administered to a subject in need thereof over the course of treatment. In some embodiments, the treatment regimen may change such that other standard of care treatments (e.g., dietary changes, changes in exercise habits, eliminating or reducing alcohol intake) that are administered as a combination therapy to treat a subject in need thereof are further modulated according to methods and guidelines known in the art.

### EXAMPLES

### Example 1. NAFLD Targets Inhibited by siRNA

This example describes siRNA knockdown of targets identified by the inventors as targets for the treatment of NAFLD, steatosis, liver inflammation, or liver fibrosis, or for the reduction of lipid droplets in a liver cell of a subject.

Human hepatocytes (e.g., HepG2 cells) were reverse transfected with an siRNA cocktail against identified targets using lipofectamine at a final siRNA concentration of 100 nM in serum and antibiotic free Eagle's Minimum Essential Medium (EMEM). As a control, some hepatocytes were transfected with a non-targeting scrambled siRNA or were administered a vehicle-only preparation. As a positive control, some hepatocytes were transfected with an siRNA that inhibits expression of PLIN2, a protein previously shown to reduce lipid droplet formation when inhibited by an siRNA. The hepatocytes were then plated in a 96-well plate following the transfection at a density of 1×10⁴ cells per well.

The following day, the media in the 96-well plate was replaced with fresh serum and antibiotic free EMEM. About 24 hours after replacing the media, the hepatocytes were treated with 200 μM of oleic acid conjugated to bovine serum albumin in serum free EMEM for another 24 hours in order to induce lipid droplet accumulation in the cells.

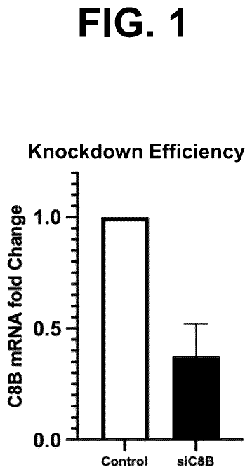

Following the application of oleic acid, changes in gene expression were analyzed. The efficiency of the siRNA knockdown was assessed by qPCR, as shown in FIG. 1 and Table 5. As seen, the siRNA was effective to reduce gene expression of the targets listed in Table 5.

TABLE 5
Knockdown Efficiency

| | | Knockdown |
|---|---|---|
| Target | siRNA SEQ ID NOs: | Efficiency |
| ABCB4 | Pooled siRNAs of SEQ ID NOs: 59, 61, 63, | + |
| | and 65 | |
| CFB | Pooled siRNAs of SEQ ID NOs: 67, 69, 71, | + |
| | and 73 | |
| C8B | Pooled siRNAs of SEQ ID NOs: 75, 77, 79, | + |
| | and 81 | |
| HGD | Pooled siRNAs of SEQ ID NOs: 99, 101, 103, | + |
| | and 105 | |
| MCEE | Pooled siRNAs of SEQ ID NOs: 107, 109, 111, | + |
| | and 113 | |
| PEX6 | Pooled siRNAs of SEQ ID NOs: 123, 125, 127, | + |
| | and 129 | |
| PRELID2 | Pooled siRNAs of SEQ ID NOs: 131, 133, 135, | + |
| | and 137 | |
| SH3PXD2A | Pooled siRNAs of SEQ ID NOs: 155, 157, 159, | + |
| | and 161 | |
| SLC16A10 | Pooled siRNAs of SEQ ID NOs: 163, 165, 167, | + |
| | and 169 | |
| TTR | Pooled siRNAs of SEQ ID NOs: 179, 181, 183, | + |
| | and 185 | |

+ = 30% or greater reduction in gene expression compared to control

### Example 2. NAFLD Targets Inhibited by siRNA

This example describes siRNA knockdown of targets HPR, RAB11A, and SLC22A25 identified by the inventors as targets for the treatment of NAFLD, steatosis, liver inflammation, or liver fibrosis, or for the reduction of lipid droplets in a liver cell of a subject.

Human hepatocytes (e.g., HepG2 cells) were reverse transfected with an siRNA cocktail against identified targets using lipofectamine at a final siRNA concentration of 50 nM in serum and antibiotic free Eagle's Minimum Essential Medium (EMEM). As a control, some hepatocytes were transfected with a non-targeting scrambled siRNA or were administered a vehicle-only preparation. As a positive control, some hepatocytes were transfected with an siRNA that inhibits expression of PLIN2, a protein previously shown to reduce lipid droplet formation when inhibited by an siRNA. The hepatocytes were then plated in a 6-well plate following the transfection at a density of 3×10⁵ cells per well.

The following day, the media in the 6-well plate was replaced with fresh serum and antibiotic free EMEM. About 24 hours after replacing the media, the hepatocytes were treated with 200 μM of oleic acid conjugated to bovine serum albumin in serum free EMEM for another 24 hours in order to induce lipid droplet accumulation in the cells.

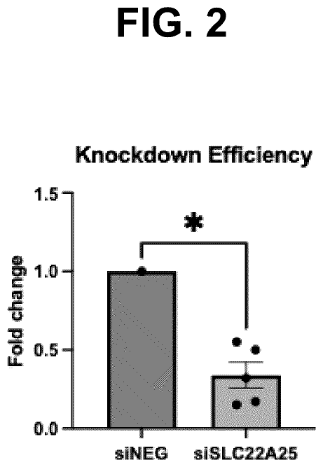

Following the application of oleic acid, changes in gene expression were analyzed. The efficiency of the siRNA knockdown was assessed by qPCR, as shown in FIG. 2 and Table 6. As seen, the siRNA was effective to reduce gene expression of the targets listed in Table 6.

TABLE 6
Knockdown Efficiency

| | | Knockdown |
|---|---|---|
| Target | siRNA SEQ ID NOs: | Efficiency |
| HPR | Pooled siRNAs of SEQ ID NOs: 91, 93, | + |
| | 95, and 97 | |
| RAB11A | Pooled siRNAs of SEQ ID NOs: 139, 141, | + |
| | 143, and 145 | |
| SLC22A25 | Pooled siRNAs of SEQ ID NOs: 171, 173, | + |
| | 175, and 177 | |

+ = 30% or greater reduction in gene expression compared to control

### Example 3. Reduction in Lipid Accumulation in Hepatocytes Treated with siRNA Against Identified Targets

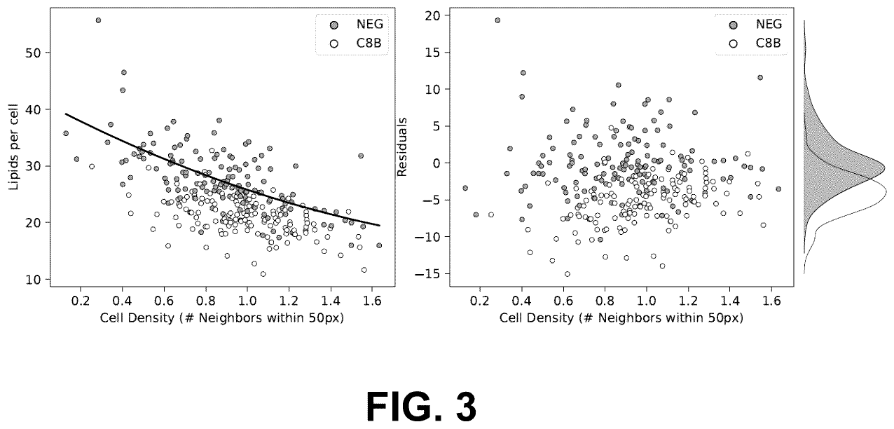

The oleic acid-induced, siRNA-treated hepatocytes described in Examples 1 and 2 were stained with Hoechst stain and Biotium LIPIDSPOT™ 488 for 1 hour for detection of nuclei and lipid droplets, respectively. Lipid droplet accumulation was measured by fluorescence microscopy, wherein the number of lipid droplets per cell were quantified. Reduced lipid droplet accumulation was assessed by comparing cell populations treated with an siRNA to a control population. The change in lipid droplet accumulation is represented by the z-score. A baseline of lipid droplet accumulation corrected for cell density is calculated in the siNEG control condition. The standard Z-test is then applied to determine if lipid droplet accumulation associated with a particular condition is significantly higher or lower than the baseline. The z-score of a particular condition can be interpreted as the number of standard deviations the mean of that treatment is away from the baseline. For a given treatment, a negative z-score reflects a decrease in lipid accumulation while a positive z-score reflects an increase in lipid accumulation. Only |z-score|>4 is interpreted as a significant change relative to the control condition (equivalent to P-value <0.05, adjusted for multiple hypothesis testing). As shown in FIG. 3 and Table 7, siRNA inhibition of the identified targets reduced lipid droplet accumulation in hepatocytes.

TABLE 7
Lipid Droplet Accumulation

| | | Lipid Droplet |
|---|---|---|
| Target | siRNA SEQ ID NOs: | Accumulation |
| ABCB4 | Pooled siRNAs of SEQ ID NOs: 59, 61, | + |
| | 63, and 65 | |
| CFB | Pooled siRNAs of SEQ ID NOs: 67, 69, | + |
| | 71, and 73 | |
| C8B | Pooled siRNAs of SEQ ID NOs: 75, 77, | ++ |
| | 79, and 81 | |
| HGD | Pooled siRNAs of SEQ ID NOs: 99, 101, | +++ |
| | 103, and 105 | |
| MCEE | Pooled siRNAs of SEQ ID NOs: 107, 109, | + |
| | 111, and 113 | |
| PEX6 | Pooled siRNAs of SEQ ID NOs: 123, 125, | + |
| | 127, and 129 | |
| PRELID2 | Pooled siRNAs of SEQ ID NOs: 131, 133, | + |
| | 135, and 137 | |
| SH3PXD2A | Pooled siRNAs of SEQ ID NOs: 155, 157, | ++ |
| | 159, and 161 | |
| SLC16A10 | Pooled siRNAs of SEQ ID NOs: 163, 165, | ++ |
| | 167, and 169 | |
| TTR | Pooled siRNAs of SEQ ID NOs: 179, 181, | ++ |
| | 183, and 185 | |

+ = z-score <−4
++ = z-score <−8
+++ = z-score <−12

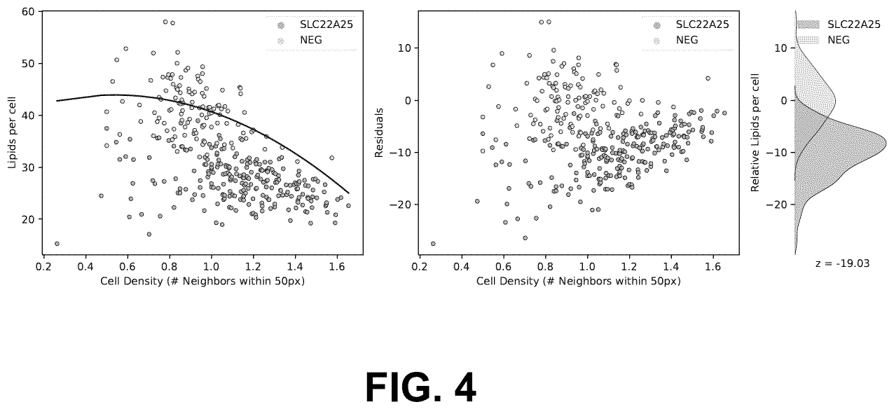

Moreover, FIG. 4 and Table 8 shows siRNA inhibition of the additionally identified targets reduced lipid droplet accumulation in hepatocytes.

TABLE 8
Lipid Droplet Accumulation

| | | Lipid |
|---|---|---|
| | | Droplet |
| | | Accu- |
| Target | siRNA SEQ ID NOs: | mulation |
| HPR | Pooled siRNAs of SEQ ID NOs: 91, 93, 95, | + |
| | and 97 | |
| RAB11A | Pooled siRNAs of SEQ ID NOs: 139, 141, | + |
| | 143, and 145 | |
| SLC22A25 | Pooled siRNAs of SEQ ID NOs: 171, 173, | +++ |
| | 175, and 177 | |

+ = z-score <−4
++ = z-score <−8
+++ = z-score <−12

### Example 4. Treating a Subject at Risk of Developing NAFLD

A subject is identified by a clinician as being at risk of developing nonalcoholic fatty liver disease (NAFLD) due to the subject's poor diet and exercise habits and the subject having >5% hepatic steatosis (e.g., 10%, 15%, 20%, 25%, 30%, 35%, 40%, or greater hepatic steatosis). The subject is then administered (e.g., intravenously or subcutaneously) a therapeutically effective amount of a pharmaceutical composition containing one or more inhibitory nucleic acid molecules (e.g., one or more siRNA molecules having a nucleotide sequence of SEQ ID NOs: 43-194). The inhibitory nucleic acid molecule is conjugated to a liver targeting moiety (e.g., an N-acetylgalactosamine (GaINAc) ligand). Following treatment (e.g., 1, 2, 3, 4, 5, 6, 7, 8, 9, 10, 11, 12, or more administrations of the pharmaceutical composition over the course of 3, 6, 9, or 12 months), hepatic steatosis is measured again. The subject's hepatic steatosis is assessed and compared to the level of hepatic steatosis prior to treatment.

### Example 5. Treating a Subject Having NAFLD

Magnetic resonance imaging (MRI) of a subject's liver indicates that the subject indicates >5% (e.g., 5.1%, 10%, 15%, 20%, 25%, 30%, 40%, or greater) liver steatosis, thereby indicating that the subject has NAFLD. The subject is then treated for NAFLD by administering (e.g., intravenously or subcutaneously) a dose (e.g., a therapeutic dose or sub-therapeutic dose) of one or more inhibitory nucleic acid molecules (e.g., one or more siRNA molecules having a nucleotide sequence of SEQ ID NOs: 43-194). Following treatment (e.g., 1, 2, 3, 4, 5, 6, 7, 8, 9, 10, 11, 12, 13, 14, 15, 16, 17, 18, 19, 20, or more administrations of the inhibitory nucleic acid molecule), a second MRI of the patient's liver is performed and the subject's liver steatosis status relative to the MRI prior to treatment is assessed.

### Example 6. Reducing the Risk of NASH in a Subject Having NAFLD

A subject previously diagnosed with NAFL has about 16% liver steatosis (i.e., 16% of the hepatocytes in the liver exhibit lipid accumulation) as determined by MRI. A magnetic resonance elastography (MRE) also determines that the subject does not have any measurable fibrosis in the liver. The subject is then treated with a therapeutically effective amount of an siRNA (e.g., an siRNA molecule of Table 4) formulated as a pharmaceutical composition. Following treatment with the siRNA (e.g., 1, 2, 3, or 4, administrations of the siRNA over the course of one year), an MRI again determines the subject's hepatic steatosis to be unchanged and an MRE again determines no measurable fibrosis in the liver, thereby indicating that treatment is working to reduce progression. The subject continues treatment and their NAFL does not progress to NASH. An additional MRI one year later determines the subject's hepatic steatosis to be 12%, thereby indicating that the treatment is working to reduce steatosis and reduce the risk of progression of NAFLD.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of treating a human subject identified as having or at risk of developing nonalcoholic fatty liver disease (NAFLD), the method comprising the step of administering to the subject an inhibitor of ATP binding cassette subfamily B member 4 (ABCB4), complement C8 beta chain (C8B), homogentisate 1,2-dioxygenase (HGD), methylmalonyl-CoA epimerase (MCEE), SH3 and PX domains 2A (SH3PXD2A), Solute carrier family 16 member 10 (SLC16A10), transthyretin (TTR), haptoglobin-related protein (HPR), peroxisomal biogenesis factor 6 (PEX6), RAB11A, Member RAS Oncogene Family (RAB11 A), or solute carrier family 22 member 25 (SLC22A25).

2. (canceled)

3. The method of claim 1, wherein (i) the subject has >5% liver steatosis, or (ii) the subject is at risk of developing NAFLD and has S 5% liver steatosis.

4. (canceled)

5. The method of claim 1, wherein the NAFLD is nonalcoholic fatty liver (NAFL) or nonalcoholic steatohepatitis (NASH), or wherein the inhibitor is delivered to a hepatocyte (HC) in the subject.

6. (canceled)

7. A method of treating liver steatosis, liver inflammation, or liver fibrosis in a human subject in need thereof, the method comprising the step of administering to the subject an inhibitor of ABCB4, C8B, HGD, MCEE, SH3PXD2A, SLC16A10, TTR, HPR, PEX6, RAB11A, or SLC22A25.

8-9. (canceled)

10. A method of reducing the number of lipid droplets or reducing lipid droplet area in a liver cell of a human subject, the method comprising the step of administering to the subject an inhibitor of ABCB4, C8B, HGD, MCEE, SH3PXD2A, SLC16A10, TTR, HPR, PEX6, RAB11A, or SLC22A25.

11. (canceled)

12. The method of claim 10, wherein the liver cell is a hepatocyte.

13. The method of claim 1, wherein the inhibitor is an agent listed in Table 2.

14. The method of claim 1, wherein the inhibitor is
a small interfering RNA (siRNA).

15-21. (canceled)

22. The method of claim 1, wherein the inhibitor is a small molecule listed in Table 3.

23. The method of claim 1, wherein the inhibitor is a protein or a peptide listed in Table 3.

24. The method of claim 7, wherein the inhibitor is an agent listed in Table 2.

25. The method of claim 7, wherein the inhibitor is an siRNA.

26. The method of claim 7, wherein the inhibitor is a small molecule listed in Table 3.

27. The method of claim 7, wherein the inhibitor is a protein or a peptide listed in Table 3.

28. The method of claim 10, wherein the inhibitor is an agent listed in Table 2.

29. The method of claim 10, wherein the inhibitor is an siRNA.

30. The method of claim 10, wherein the inhibitor is a small molecule listed in Table 3.

31. The method of claim 10, wherein the inhibitor is a protein or a peptide listed in Table 3.


SEQUENCE LISTING (194 sequences)

SEQ ID NO: 1 - RNA, 6513 nt, Homo sapiens
  source 1..6513: mol_type = mRNA
atgaaagactccgaagggccccagaggcccccgctgtgttttttatctacattgctttcccagaaggttcctgagaagtcagacgctgtgcttcgctgcataatatctggtcagcccaagccagaggtaacttggtataagaatggtcaggccatcgatgggagtggcattatttccaactatgaattctttgagaatcagtatattcatgtgttacatctctcttgctgtaccaaaaatgatgctgctgtctatcaaatctcggctaaaaactcttttggaatgatctgttgttctgcttccgttgaggttgagtgctcatcagagaacccacaattgtctcctaacctggaagatgacagggacaggggttggaaacatgaaacagggacacatgaagaagaaagggcaaatcagattgatgagaaggaacatccttataaggaagaagaaagcatctccccgggcactcccaggtcagctgactcctccccctccaaatccaaccattcactctccctccagtcattgggcaatcttgacattagtgtgtccagttctgaaaatcctttgggtgttaaaggaacaaggcacactggagaggcttatgatccaagtaacacagaagaaattgcaaatgggttgctttttcttaattcaagtcatatttatgaaaaacaagacagatgttgccacaagacagtgcattccatggcatcaaagttcacggatggtgacctgaacaatgatggtcctcatgatgaaggcttacgctctagtcagcaaaatcccaaagtacagaaatacattagcttcagcctcccgctatctgaggcaactgcacacatttacccaggtgacagtgccgtggccaacaaacaacccagcccacagctttccagtgaagactctgacagtgactatgaactttgcccagagataaccctaacctacaccgaggagttttcagatgatgacctggagtatctggaatgttctgatgttatgacggattactctaatgcagtttggcaaaggaacctgctggggactgagcatgtttttttattagaaagcgatgacgaagagatggaattcggtgagcattgcctgggtgggtgtgagcatttcctcagtggaatgggttgtgggtctcgggtgtcgggtgacgctgggcctatggttgccactgctggcttctgtggtcatcactcacaaccccaagaagttggggtgaggagcagcagagtctccaagcacggtccctcatccccacaaacagggatgactctcattttgggacctcaccaggatggaacgtcttcagtgacagaacaggggagatataaactccccactgctcccgaggctgctgaaaatgattatccaggaattcaaggagaaaccagagacagccaccaagcaagagaggaatttgccagtgacaatctgctcaacatggatgaatcagtaagagagacagagatgaagctcttgtctggtgagtcagaaaactcagggatgagccagtgttgggagacggcagctgacaagagagtggggggaaaggacttatggagcaagaggggttcaaggaaatctgccagggtgaggcagccgggaatgaagggaaatcccaagaagccgaatgccaacctgagagaaagtacaacagaaggtacccttcatctctgctctgccaaagaatctgctgagcccccactaacccagagtgataaaagagagacttctcacaccacagcagcagcgactggtcggagttcccatgctgatgcaagagaatgtgctatttcaacccaggcagagcaagaagcaaaaacccttcaaacttcaacagactcagtctccaaagaaggcaacacaaattgcaagggagaaggcatgcaagttaatactctatttgaaacaagccaggttccagactggagtgatcctcctcaggtacaagttcaggaaacagtcagagagacaatctcttgcagccagatgccagctttctcagagcctgctggggaggagtccccattcactgggaccacaacaatttccttctcaaacttaggaggggtccacaaggaaaatgcatcattagctcaacactcggaggtcaaaccctgtacctgtggtccacagcatgaagaaaaacaagacagagatggcaacatacctgacaatttcagggaagacctaaaatatgagcagagcatctcagaagccaatgatgagactatgtccccaggtgtgttctcaaggcatctccccaaggatgctcgtgctgacttcagggagcctgtggctgtctctgttgcttcccctgaacccacagatactgccctcaccctggaaaatgtgtgtgatgagccaagggacagagaagcagtgtgtgcaatggagtgttttgaggctggtgaccaaggaacgtgttttgataccatagattctcttgttgggagaccagttgataaatattcgcctcaagaaatttgctctgtagatacggaactggcagaaggtcaaaacaaagtatctgatttatgttcttctaatgacaagacactggaagtcttttttcagacacaagtgtctgagacttcagtgtctacgtgcaaaagcagcaaggacggcaactcagtcatgtcccctctttttaccagtactttcaccttgaacatttcacacacagctagtgaaggtgccacaggagaaaatctagccaaggtggagaattccacctacccactggcctccacagtacatgctggccaggagcagccaagccccagcaactcaggagggcttgatgaaacacagctcctttcttctgagaacaatcctttagtgcaatttaaagaaggaggtgacaagagccccagtcctagtgccgcagacaccacagccacaccagccagttatagttcaattgtgagttttccttgggagaagccaacaacattaactgctaataatgagtgctttcaagcgaccagggagactgaggacacatcaactgttaccattgccaccgaagtccacccagccaaataccttgctgtgtcaattcctgaggacaagcatgcaggtggcactgaggagaggttccctcgtgcatcccatgaaaaggtttcccaatttccttcccaagtgcagttggatcatattttaagtggtgctaccatcaaatctacaaaagagctactttgcagggcacccagtgtgccaggagtcccacaccatgtcctgcagctcccagagggagagggtttctgcagtaattcccctcttcaggttgataacctgtctggagataagagccagactgtggacagagcagactttaggagctatgaagagaatttccaagaaagaggaagtgaaacaaagcagggggtccagcagcagagcctgtcccagcagggttctctttctgcacctgatttccaacaaagtttgcctacgacatctgctgcacaagaggaaagaaacttggtgcccacggcccactcacccgcaagctctagggaaggagcagggcagcgctcaggttgggggacgagggtctccgtggtggctgaaactgctggggaagaagacagtcaggctctgagcaacgttccatctctctctgatatccttttggaagagtctaaagaatatagacctggaaattgggaggcaggcaacaagctgaagattataactctagaggcttccgcttctgaaatctggccaccacgacaactgacaaattctgagagcaaggcatcagacggtggtctcataattcctgacaaggtctgggctgtacctgatagtctaaaggcagatgctgttgtgcctgaattggccccctctgaaatagcagcattggctcacagtccagaggatgctgagtcagcccttgctgatagcagagaaagccataaaggcgaagagcccaccatcagtgtacattggagaagtctttcttcccggggtttcagccaacccagactcctggagtcatccgtggaccctgtagatgaaaaggagttatctgtcacagattcactgtcagcggcttctgaaactggagggaaggaaaatgttaacaatgtgagtcaagaccaggaggaaaaacaactcaagatggatcacactgccttctttaaaaagtttctgacctgccctaaaatcctagagtcctctgtagatcccattgatgagataagtgtgatagagtacaccagggctggaaaaccagagccctctgaaaccacaccacagggcgccagagaagggggtcaatcaaatgacggaaacatgggccacgaagcggaaatccagccggccattttgcaagttccatgtctccagggaaccattctgagtgaaaatagaatcagcagaagccaagaaggcagtatgaagcaggaggctgaacaaattcaacctgaggaggcaaaaactgccatttggcaagtcctgcaacccagcgaaggcggtgaaagaattccaagtggatgtagcataggccaaatacaagaaagcagtgatgggagcttaggggaggctgagcaaagcaaaaaggacaaagcagaattgatttcccccacttcacctctttctagttgtcttccaataatgactcacgcttctcttggggttgacacgcacaactccacaggccaaattcatgacgtccctgaaaatgacatagttgagcccagaaagcgtcagtatgtgtttcctgtttcacagaaaaggggaactattgagaatgagcgtgggaaacctttgccctcttctcctgatcttaccaggttcccttgtacttcatctcctgaaggaaatgtcacagactttttgataagccacaaaatggaggaacctaaaatagaggtgcttcaaattggggaaaccaaacccccaagctcatctagctcctcagcgaagaccttggcatttatttcaggagaacgtgagttagagaaagcccctaaattactgcaggatccatgtcaaaagggcaccctgggctgtgcgaaaaagtccagggagagagagaagtccctggaagcccgagcaggcaaatcgccagggaccctcacagcagtgacggggtcagaggaggtcaagaggaagccagaagccccaggcagtggacatttagctgagggagtaaagaagaaaattttgtccagggtggcagcactgaggctgaaactggaagaaaaggaaaatatcagaaagaactcagcctttcttaaaaagatgcccaaactcgaaacatcattatcacacacagaagagaaacaagacccaaaaaagccatcttgcaaaagagaaggaagagctccagtattactgaaaaagatccaagctgagatgttccctgaacactctggaaatgtaaaattaagctgccaatttgcagaaattcatgaagattctactatctgctggacaaaagattcaaagtccatagcccaagtgcagagaagtgcaggggacaactccactgtttcctttgccatcgtgcaagccagtccgaaggaccagggactctattactgctgcatcaagaacagctacggaaaagtgactgctgaatttaacctcacagctgaagttctcaaacagctgtcaagtcgccaggatactaaaggatgtgaagagattgaattcagccaactcatcttcaaagaagacttcctccatgacagctactttgggggccgcctgcgtggtcagatcgccacggaggagctgcactttggagaaggggttcaccgcaaagccttccgcagcacagtgatgcacggcctcatgcctgtcttcaaacctggccatgcctgtgtgcttaaggtgcacaatgccattgcctatgggaccagaaataatgatgagctcatccaaaggaactacaaactcgctgcccaggaatgctatgttcaaaatactgccaggtattatgccaagatctacgctgctgaagcacagcctctggaaggctttggagaagtacctgagatcattcctatttttcttatccatcggcctgagaacaatatcccgtatgctacagtggaggaggagctgattggagaatttgtgaagtattccatcagggatgggaaagaaataaacttcttgagaagagaatcagaagctggtcagaaatgttgcaccttccagcactgggtgtaccagaaaacaagtggctgcctcctggtgacggacatgcaaggtgtaggaatgaagctaactgacgttggcatagcaacgctggctaaagggtacaagggatttaaaggcaactgttccatgaccttcattgatcagtttaaagcactacaccagtgtaacaagtattgcaaaatgctgggactgaaatcccttcaaaacaacaaccagaaacagaagcagccgagcattgggaaaagcaaagttcaaacaaactctatgacaataaagaaggcagggcctgagaccccaggcgaaaagaaaacctaa

SEQ ID NO: 2 - protein, 2170 aa, Homo sapiens
MKDSEGPQRPPLCFLSTLLSQKVPEKSDAVLRCIISGQPKPEVTWYKNGQAIDGSGIISNYEFFENQYIHVLHLSCCTKNDAAVYQISAKNSFGMICCSASVEVECSSENPQLSPNLEDDRDRGWKHETGTHEEERANQIDEKEHPYKEEESISPGTPRSADSSPSKSNHSLSLQSLGNLDISVSSSENPLGVKGTRHTGEAYDPSNTEEIANGLLFLNSSHIYEKQDRCCHKTVHSMASKFTDGDLNNDGPHDEGLRSSQQNPKVQKYISFSLPLSEATAHIYPGDSAVANKQPSPQLSSEDSDSDYELCPEITLTYTEEFSDDDLEYLECSDVMTDYSNAVWQRNLLGTEHVFLLESDDEEMEFGEHCLGGCEHFLSGMGCGSRVSGDAGPMVATAGFCGHHSQPQEVGVRSSRVSKHGPSSPQTGMTLILGPHQDGTSSVTEQGRYKLPTAPEAAENDYPGIQGETRDSHQAREEFASDNLLNMDESVRETEMKLLSGESENSGMSQCWETAADKRVGGKDLWSKRGSRKSARVRQPGMKGNPKKPNANLRESTTEGTLHLCSAKESAEPPLTQSDKRETSHTTAAATGRSSHADARECAISTQAEQEAKTLQTSTDSVSKEGNTNCKGEGMQVNTLFETSQVPDWSDPPQVQVQETVRETISCSQMPAFSEPAGEESPFTGTTTISFSNLGGVHKENASLAQHSEVKPCTCGPQHEEKQDRDGNIPDNFREDLKYEQSISEANDETMSPGVFSRHLPKDARADFREPVAVSVASPEPTDTALTLENVCDEPRDREAVCAMECFEAGDQGTCFDTIDSLVGRPVDKYSPQEICSVDTELAEGQNKVSDLCSSNDKTLEVFFQTQVSETSVSTCKSSKDGNSVMSPLFTSTFTLNISHTASEGATGENLAKVENSTYPLASTVHAGQEQPSPSNSGGLDETQLLSSENNPLVQFKEGGDKSPSPSAADTTATPASYSSIVSFPWEKPTTLTANNECFQATRETEDTSTVTIATEVHPAKYLAVSIPEDKHAGGTEERFPRASHEKVSQFPSQVQLDHILSGATIKSTKELLCRAPSVPGVPHHVLQLPEGEGFCSNSPLQVDNLSGDKSQTVDRADFRSYEENFQERGSETKQGVQQQSLSQQGSLSAPDFQQSLPTTSAAQEERNLVPTAHSPASSREGAGQRSGWGTRVSVVAETAGEEDSQALSNVPSLSDILLEESKEYRPGNWEAGNKLKIITLEASASEIWPPRQLTNSESKASDGGLIIPDKVWAVPDSLKADAVVPELAPSEIAALAHSPEDAESALADSRESHKGEEPTISVHWRSLSSRGFSQPRLLESSVDPVDEKELSVTDSLSAASETGGKENVNNVSQDQEEKQLKMDHTAFFKKFLTCPKILESSVDPIDEISVIEYTRAGKPEPSETTPQGAREGGQSNDGNMGHEAEIQPAILQVPCLQGTILSENRISRSQEGSMKQEAEQIQPEEAKTAIWQVLQPSEGGERIPSGCSIGQIQESSDGSLGEAEQSKKDKAELISPTSPLSSCLPIMTHASLGVDTHNSTGQIHDVPENDIVEPRKRQYVFPVSQKRGTIENERGKPLPSSPDLTRFPCTSSPEGNVTDFLISHKMEEPKIEVLQIGETKPPSSSSSSAKTLAFISGERELEKAPKLLQDPCQKGTLGCAKKSREREKSLEARAGKSPGTLTAVTGSEEVKRKPEAPGSGHLAEGVKKKILSRVAALRLKLEEKENIRKNSAFLKKMPKLETSLSHTEEKQDPKKPSCKREGRAPVLLKKIQAEMFPEHSGNVKLSCQFAEIHEDSTICWTKDSKSIAQVQRSAGDNSTVSFAIVQASPKDQGLYYCCIKNSYGKVTAEFNLTAEVLKQLSSRQDTKGCEEIEFSQLIFKEDFLHDSYFGGRLRGQIATEELHFGEGVHRKAFRSTVMHGLMPVFKPGHACVLKVHNAIAYGTRNNDELIQRNYKLAAQECYVQNTARYYAKIYAAEAQPLEGFGEVPEIIPIFLIHRPENNIPYATVEEELIGEFVKYSIRDGKEINFLRRESEAGQKCCTFQHWVYQKTSGCLLVTDMQGVGMKLTDVGIATLAKGYKGFKGNCSMTFIDQFKALHQCNKYCKMLGLKSLQNNNQKQKQPSIGKSKVQTNSMTIKKAGPETPGEKKT

SEQ ID NO: 3 - RNA, 1011 nt, Homo sapiens
  source 1..1011: mol_type = mRNA
atgaaaattgacatccatagtcatattctaccaaaagaatggccagatctaaaaaagaggtttggctacggaggctgggtgcagctccaacaccacagcaagggagaagcaaagttgttgaaagatgggaaagtcttcagagtggtgcgagagaattgctgggatccagaagttcgtattagagaaatggaccaaaaaggagtaacagtgcaagccctttccacagttcctgtcatgtttagctactgggccaaacctgaggacactttaaacctgtgccagcttttaaacaacgaccttgccagcaccgttgtgagctaccccaggaggttcgtgggtctggggacgttgcccatgcaggcccctgagctggcggtcaaggagatggagcgctgtgtgaaagagctgggctttcccggggtccaaattggcacccacgtcaacgagtgggacctgaacgcgcaggagctctttcctgtctatgcggcagccgaaaggctgaagtgttccctgttcgtgcatccctgggacatgcagatggatggacgaatggccaaatactggctcccttggcttgtaggaatgccagcagagaccaccatagccatttgctccatgatcatgggtggagtatttgagaagtttcccaaactgaaagtgtgtttcgcacatggtggtggtgccttccccttcacagtgggaagaatctcccatggattcagcatgcgcccagatctgtgtgcccaggacaaccccatgaacccgaagaaataccttggttccttttacacagatgctttggttcatgatcctctgtccctcaagctgttaacagatgtcataggaaaggataaagtcattttgggaaccgattacccctttccactaggtgagctggaacctgggaaactaatagagtccatggaagaatttgatgaagaaacaaagaataaactcaaagccggcaatgccctggcatttttgggtcttgagagaaaacaatttgaatga

SEQ ID NO: 4 - protein, 336 aa, Homo sapiens
MKIDIHSHILPKEWPDLKKRFGYGGWVQLQHHSKGEAKLLKDGKVFRVVRENCWDPEVRIREMDQKGVTVQALSTVPVMFSYWAKPEDTLNLCQLLNNDLASTVVSYPRRFVGLGTLPMQAPELAVKEMERCVKELGFPGVQIGTHVNEWDLNAQELFPVYAAAERLKCSLFVHPWDMQMDGRMAKYWLPWLVGMPAETTIAICSMIMGGVFEKFPKLKVCFAHGGGAFPFTVGRISHGFSMRPDLCAQDNPMNPKKYLGSFYTDALVHDPLSLKLLTDVIGKDKVILGTDYPFPLGELEPGKLIESMEEFDEETKNKLKAGNALAFLGLERKQFE

SEQ ID NO: 5 - RNA, 3699 nt, Homo sapiens
  source 1..3699: mol_type = mRNA
atggatcttgaggcggcaaagaacggaacagcctggcgccccacgagcgcggagggcgactttgaactgggcatcagcagcaaacaaaaaaggaaaaaaacgaagacagtgaaaatgattggagtattaacattgtttcgatactccgattggcaggataaattgtttatgtcgctgggtaccatcatggccatagctcacggatcaggtctccccctcatgatgatagtatttggagagatgactgacaaatttgttgatactgcaggaaacttctcctttccagtgaacttttccttgtcgctgctaaatccaggcaaaattctggaagaagaaatgactagatatgcatattactactcaggattgggtgctggagttcttgttgctgcctatatacaagtttcattttggactttggcagctggtcgacagatcaggaaaattaggcagaagttttttcatgctattctacgacaggaaataggatggtttgacatcaacgacaccactgaactcaatacgcggctaacagatgacatctccaaaatcagtgaaggaattggtgacaaggttggaatgttctttcaagcagtagccacgttttttgcaggattcatagtgggattcatcagaggatggaagctcacccttgtgataatggccatcagccctattctaggactctctgcagccgtttgggcaaagatactctcggcatttagtgacaaagaactagctgcttatgcaaaagcaggcgccgtggcagaagaggctctgggggccatcaggactgtgatagctttcgggggccagaacaaagagctggaaaggtatcagaaacatttagaaaatgccaaagagattggaattaaaaaagctatttcagcaaacatttccatgggtattgccttcctgttaatatatgcatcatatgcactggccttctggtatggatccactctagtcatatcaaaagaatatactattggaaatgcaatgacagtttttttttcaatcctaattggagctttcagtgttggccaggctgccccatgtattgatgcttttgccaatgcaagaggagcagcatatgtgatctttgatattattgataataatcctaaaattgacagtttttcagagagaggacacaaaccagacagcatcaaagggaatttggagttcaatgatgttcacttttcttacccttctcgagctaacgtcaagatcttgaagggcctcaacctgaaggtgcagagtgggcagacggtggccctggttggaagtagtggctgtgggaagagcacaacggtccagctgatacagaggctctatgaccctgatgagggcacaattaacattgatgggcaggatattaggaactttaatgtaaactatctgagggaaatcattggtgtggtgagtcaggagccggtgctgttttccaccacaattgctgaaaatatttgttatggccgtggaaatgtaaccatggatgagataaagaaagctgtcaaagaggccaacgcctatgagtttatcatgaaattaccacagaaatttgacaccctggttggagagagaggggcccagctgagtggtgggcagaagcagaggatcgccattgcacgtgccctggttcgcaaccccaagatccttctgctggatgaggccacgtcagcattggacacagaaagtgaagctgaggtacaggcagctctggataaggccagagaaggccggaccaccattgtgatagcacaccgactgtctacggtccgaaatgcagatgtcatcgctgggtttgaggatggagtaattgtggagcaaggaagccacagcgaactgatgaagaaggaaggggtgtacttcaaacttgtcaacatgcagacatcaggaagccagatccagtcagaagaatttgaactaaatgatgaaaaggctgccactagaatggccccaaatggctggaaatctcgcctatttaggcattctactcagaaaaaccttaaaaattcacaaatgtgtcagaagagccttgatgtggaaaccgatggacttgaagcaaatgtgccaccagtgtcctttctgaaggtcctgaaactgaataaaacagaatggccctactttgtcgtgggaacagtatgtgccattgccaatggggggcttcagccggcattttcagtcatattctcagagatcatagcgatttttggaccaggcgatgatgcagtgaagcagcagaagtgcaacatattctctttgattttcttatttctgggaattatttctttttttactttcttccttcagggtttcacgtttgggaaagctggcgagatcctcaccagaagactgcggtcaatggcttttaaagcaatgctaagacaggacatgagctggtttgatgaccataaaaacagtactggtgcactttctacaagacttgccacagatgctgcccaagtccaaggagccacaggaaccaggttggctttaattgcacagaatatagctaaccttggaactggtattatcatatcatttatctacggttggcagttaaccctattgctattagcagttgttccaattattgctgtgtcaggaattgttgaaatgaaattgttggctggaaatgccaaaagagataaaaaagaactggaagctgctggaaagattgcaacagaggcaatagaaaatattaggacagttgtgtctttgacccaggaaagaaaatttgaatcaatgtatgttgaaaaattgtatggaccttacagggtgttttctgcaattgtatttggtgcagtggctctaggacatgccagttcatttgctccagactatgctaaagctaagctgtctgcagcccacttattcatgctgtttgaaagacaacctctgattgacagctacagtgaagaggggctgaagcctgataaatttgaaggaaatataacatttaatgaagtcgtgttcaactatcccacccgagcaaacgtgccagtgcttcaggggctgagcctggaggtgaagaaaggccagacactagccctggtgggcagcagtggctgtgggaagagcacggtggtccagctcctggagcggttctacgaccccttggcggggacagtgcttctcgatggtcaagaagcaaagaaactcaatgtccagtggctcagagctcaactcggaatcgtgtctcaggagcctatcctatttgactgcagcattgccgagaatattgcctatggagacaacagccgggttgtatcacaggatgaaattgtgagtgcagccaaagctgccaacatacatcctttcatcgagacgttaccccacaaatatgaaacaagagtgggagataaggggactcagctctcaggaggtcaaaaacagaggattgctattgcccgagccctcatcagacaacctcaaatcctcctgttggatgaagctacatcagctctggatactgaaagtgaaaaggttgtccaagaagccctggacaaagccagagaaggccgcacctgcattgtgattgctcaccgcctgtccaccatccagaatgcagacttaatagtggtgtttcagaatgggagagtcaaggagcatggcacgcatcagcagctgctggcacagaaaggcatctatttttcaatggtcagtgtccaggctgggacacagaacttatga

SEQ ID NO: 6 - protein, 1232 aa, Homo sapiens
MDLEAAKNGTAWRPTSAEGDFELGISSKQKRKKTKTVKMIGVLTLFRYSDWQDKLFMSLGTIMAIAHGSGLPLMMIVFGEMTDKFVDTAGNFSFPVNFSLSLLNPGKILEEEMTRYAYYYSGLGAGVLVAAYIQVSFWTLAAGRQIRKIRQKFFHAILRQEIGWFDINDTTELNTRLTDDISKISEGIGDKVGMFFQAVATFFAGFIVGFIRGWKLTLVIMAISPILGLSAAVWAKILSAFSDKELAAYAKAGAVAEEALGAIRTVIAFGGQNKELERYQKHLENAKEIGIKKAISANISMGIAFLLIYASYALAFWYGSTLVISKEYTIGNAMTVFFSILIGAFSVGQAAPCIDAFANARGAAYVIFDIIDNNPKIDSFSERGHKPDSIKGNLEFNDVHFSYPSRANVKILKGLNLKVQSGQTVALVGSSGCGKSTTVQLIQRLYDPDEGTINIDGQDIRNFNVNYLREIIGVVSQEPVLFSTTIAENICYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTVRNADVIAGFEDGVIVEQGSHSELMKKEGVYFKLVNMQTSGSQIQSEEFELNDEKAATRMAPNGWKSRLFRHSTQKNLKNSQMCQKSLDVETDGLEANVPPVSFLKVLKLNKTEWPYFVVGTVCAIANGGLQPAFSVIFSEIIAIFGPGDDAVKQQKCNIFSLIFLFLGIISFFTFFLQGFTFGKAGEILTRRLRSMAFKAMLRQDMSWFDDHKNSTGALSTRLATDAAQVQGATGTRLALIAQNIANLGTGIIISFIYGWQLTLLLLAVVPIIAVSGIVEMKLLAGNAKRDKKELEAAGKIATEAIENIRTVVSLTQERKFESMYVEKLYGPYRVFSAIVFGAVALGHASSFAPDYAKAKLSAAHLFMLFERQPLIDSYSEEGLKPDKFEGNITFNEVVFNYPTRANVPVLQGLSLEVKKGQTLALVGSSGCGKSTVVQLLERFYDPLAGTVLLDGQEAKKLNVQWLRAQLGIVSQEPILFDCSIAENIAYGDNSRVVSQDEIVSAAKAANIHPFIETLPHKYETRVGDKGTQLSGGQKQRIAIARALIRQPQILLLDEATSALDTESEKVVQEALDKAREGRTCIVIAHRLSTIQNADLIVVFQNGRVKEHGTHQQLLAQKGIYFSMVSVQAGTQNL

SEQ ID NO: 7 - RNA, 2295 nt, Homo sapiens
  source 1..2295: mol_type = mRNA
atggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataa

SEQ ID NO: 8 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 9 - protein, 739 aa, Homo sapiens
TPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 10 - RNA, 1776 nt, Homo sapiens
  source 1..1776: mol_type = mRNA
atgaagaattccaggacatgggcttggagggcgccggtggagctatttcttctctgtgctgccctgggctgtctcagtttgcctggctccagaggtgaaaggccacattcctttgggtcaaatgcagtcaacaagagctttgctaagagcagacagatgcggagtgtggatgttaccctgatgcccattgattgtgagctgtctagttggtcctcttggaccacatgtgacccctgtcagaagaaaaggtacaggtatgcctacttgctccagccctctcagttccatggggaaccgtgcaacttctctgacaaggaagtcgaagactgtgttaccaacagaccatgcggaagtcaagtgcgatgtgaaggctttgtgtgtgcacagacaggaaggtgtgtaaaccgcagacttctttgcaatggggacaatgactgtggagaccagtcagatgaagcaaactgtagaaggatttataaaaaatgtcagcatgaaatggaccaatactggggaattggcagtctggccagtgggataaatttgttcacaaacagttttgagggcccagttcttgatcacaggtattatgcaggtggatgctccccgcattacatcctgaacacgaggtttaggaagccctacaatgtggaaagctacacgccacagacccaaggcaaatacgaattcatattaaaagagtatgaatcatactcagattttgaacgcaatgtcacagagaaaatggcaagcaagtctggtttcagttttggttttaaaatacctggaatatttgaacttggcatcagtagtcaaagtgatcgaggcaaacactatattaggagaaccaaacgattctctcatactaaaagcgtatttctgcatgcacgctctgaccttgaagtagcacattacaagctgaaacccagaagcctcatgctccattacgagttccttcagagagttaagcggctgcccctggagtacagctacggggaatacagagatctcttccgtgattttgggacccactacatcacagaggctgtgcttgggggcatttatgaatacaccctcgttatgaacaaagaggccatggagagaggagattatactcttaacaacgtccatgcctgtgccaaaaatgattttaaaattggtggtgccattgaagaggtctacgtcagtctgggtgtgtctgtaggcaaatgcagaggtattctgaatgaaataaaagacagaaacaagagggacaccatggtggaggacttggtggtcctggtacgaggaggggcaagtgagcacatcaccaccctggcataccaggagctgccgacggcggacctgatgcaggagtggggagacgctgtgcagtacaacccagccatcatcaaagttaaggtggagcctctgtatgaactagtgacagccacagattttgcctattccagcacagtgaggcagaacatgaagcaggcactggaggagttccagaaggaagttagttcctgccactgtgctccctgccaaggaaatggagtccctgtcctgaaaggatcacgctgtgactgcatctgtcctgttggatcccaaggcctagcctgtgaggtctcctatcggaagaatacccccattgatgggaagtggaattgctggtcaaattggtcttcatgctctggaagacgtaagacaagacaaaggcagtgtaacaatccacctcctcaaaatgggggtagcccctgttcaggccctgcttcagaaacacttgactgctcctag

SEQ ID NO: 11 - protein, 591 aa, Homo sapiens
MKNSRTWAWRAPVELFLLCAALGCLSLPGSRGERPHSFGSNAVNKSFAKSRQMRSVDVTLMPIDCELSSWSSWTTCDPCQKKRYRYAYLLQPSQFHGEPCNFSDKEVEDCVTNRPCGSQVRCEGFVCAQTGRCVNRRLLCNGDNDCGDQSDEANCRRIYKKCQHEMDQYWGIGSLASGINLFTNSFEGPVLDHRYYAGGCSPHYILNTRFRKPYNVESYTPQTQGKYEFILKEYESYSDFERNVTEKMASKSGFSFGFKIPGIFELGISSQSDRGKHYIRRTKRFSHTKSVFLHARSDLEVAHYKLKPRSLMLHYEFLQRVKRLPLEYSYGEYRDLFRDFGTHYITEAVLGGIYEYTLVMNKEAMERGDYTLNNVHACAKNDFKIGGAIEEVYVSLGVSVGKCRGILNEIKDRNKRDTMVEDLVVLVRGGASEHITTLAYQELPTADLMQEWGDAVQYNPAIIKVKVEPLYELVTATDFAYSSTVRQNMKQALEEFQKEVSSCHCAPCQGNGVPVLKGSRCDCICPVGSQGLACEVSYRKNTPIDGKWNCWSNWSSCSGRRKTRQRQCNNPPPQNGGSPCSGPASETLDCS

SEQ ID NO: 12 - protein, 559 aa, Homo sapiens
ERPHSFGSNAVNKSFAKSRQMRSVDVTLMPIDCELSSWSSWTTCDPCQKKRYRYAYLLQPSQFHGEPCNFSDKEVEDCVTNRPCGSQVRCEGFVCAQTGRCVNRRLLCNGDNDCGDQSDEANCRRIYKKCQHEMDQYWGIGSLASGINLFTNSFEGPVLDHRYYAGGCSPHYILNTRFRKPYNVESYTPQTQGKYEFILKEYESYSDFERNVTEKMASKSGFSFGFKIPGIFELGISSQSDRGKHYIRRTKRFSHTKSVFLHARSDLEVAHYKLKPRSLMLHYEFLQRVKRLPLEYSYGEYRDLFRDFGTHYITEAVLGGIYEYTLVMNKEAMERGDYTLNNVHACAKNDFKIGGAIEEVYVSLGVSVGKCRGILNEIKDRNKRDTMVEDLVVLVRGGASEHITTLAYQELPTADLMQEWGDAVQYNPAIIKVKVEPLYELVTATDFAYSSTVRQNMKQALEEFQKEVSSCHCAPCQGNGVPVLKGSRCDCICPVGSQGLACEVSYRKNTPIDGKWNCWSNWSSCSGRRKTRQRQCNNPPPQNGGSPCSGPASETLDCS

SEQ ID NO: 13 - RNA, 3087 nt, Homo sapiens
  source 1..3087: mol_type = mRNA
atgatgtttccatggaaacagttgatcctgctttcattcattggctgcttaggaggtgagcttctcttacaaggccctgtatttatcaaagaacccagcaacagcattttccctgttggttcagaagataaaaaaataactttgcattgtgaagcaagaggcaatccatcacctcattacagatggcagctgaatggaagtgatattgatatgagtatggaacatcgttataagttgaatggaggaaatcttgtggttattaatcccaacagaaattgggatacaggaacttaccaatgttttgcaacaaattcacttggaacaattgtcagcagagaagccaaacttcagtttgcctatcttgaaaattttaaaaccaaaatgaggagtacagtgtctgtgcgtgaaggccagggagttgtgctgctctgcggccccccaccacactctggagaactgtcatatgcttggatcttcaatgaatacccatcgtttgttgaagaagatagtcggagatttgtctcccaggagacagggcacctctacatatctaaggtggagccgtctgatgtgggaaattacacatgtgtggtgacaagtatggtgacaaatgcccgagtgctgggctctccaactcctttggtgctacgttctgatggtgtgatgggtgaatatgaacctaaaatagaagttcagtttccagaaactcttccagcagctaaaggttcgactgtgaaattggaatgttttgcccttggaaatcccatacctcagattaattggagaagaagtgatgggctgccattttccagcaaaattaaattaaggaagttcagtggtgtgcttgaaatccccaacttccaacaggaagatgcaggttcctatgaatgcattgctgagaattcacgaggaaaaaatgttgccagagggcgtctcacttactatgcaaagccccattgggttcaactcataaaggatgtggaaatagccgtggaggacagtctttattgggaatgcagggcaagcggcaagcccaagccttcctaccgatggctgaaaaatggagcagccctggtgctagaggagagaacacagatagaaaatggtgcccttacaatatcaaacctaagtgtgactgattctggcatgttccaatgcatagcagaaaacaaacatggccttgtttattccagtgctgagctcaaagttgttgcttctgctccagatttttcaaagaatccaatgaagaagttggttcaggtgcaggtgggcagcctggtcagcttggattgtaaacccagagcctccccaagggcactctcttcctggaagaagggggatgtgagcgtgcaggagcatgaaagaatttctttgttaaacgatggaggactcaaaatagccaatgtgactaaagctgatgctggaacttacacctgcatggcagaaaaccagtttgggaaagcaaatggcacaacacatttggttgttacggaaccaacaagaataactttggcaccatctaacatggatgtttctgttggtgaaagcgtcatattgccctgccaggtacaacatgacccgctgttagacatcatctttacctggtatttcaatggggcccttgcagattttaagaaagatggatctcactttgagaaagttggtgggagttcatctggtgatttaatgatcagaaacattcagctgaaacacagtgggaaatatgtttgtatggtgcaaacgggggtggacagtgtttcatctgctgctgacctcatagtaagaggttcacctggaccaccagaaaatgtgaaggtagatgaaattacagacacaacagcccaactctcttggaaagaaggtaaagacaaccatagcccagttatatcctattctatccaggctcggacacctttctccgtgggttggcaaaccgtcacaacagtgcctgaggtcatcgatgggaagacgcacacagccactgtagttgagttaaacccatgggtggaatatgaatttcgggttgtagccagtaacaaaattggaggtggagaaccaagtttaccctcagaaaaagtaagaactgaagaggcagttccagaagtgcctccttctgaagtcaatggaggaggcggaagccggtctgaacttgtgataacctgggatccagtccctgaagaactacagaatggtgaaggttttgggtatgttgttgctttccgccctcttggggttaccacctggatccagacagtggtgacatcccctgacaccccaagatatgtctttaggaatgaaagcatcgtgccatattcaccatatgaagttaaagtgggtgtttataataacaaaggtgaaggaccatttagcccagtgacaacagtgttctctgcagaagaagagcctacagtggccccatctcaagtctctgcaaatagcctatcttcctcagaaattgaggtttcatggaacaccattccttggaagttgagcaatggacatttactgggctatgaggtgcggtactggaatgggggtggaaaggaggaatcatccagtaagatgaaagtggcaggaaatgagacatcagccagactacggggcctgaagagcaacctggcctattacacggctgtccgggcttacaacagtgccggcgctgggccttttagcgccacagttaatgtaaccaccaagaaaacgcctcccagtcagccaccaggaaatgttgtttggaatgccacagacactaaagtgttacttaattgggagcaagttaaagccatggagaatgagtcagaagtaacaggatataaagttttctataggactagcagtcaaaataacgtacaagtactgaacacaaataaaacttcagctgaacttgtgctgcccattaaagaggactacattattgaagtcaaggccacaacagatggaggggatgggaccagtagtgaacagatcaggattccacgaataaccagtatggatgcaagaggatccacttcagccatctcgaatgtccaccctatgtcaagttatatgcctatagtactgttcttaattgtatatgtcctgtggtga

SEQ ID NO: 14 - protein, 1028 aa, Homo sapiens
MMFPWKQLILLSFIGCLGGELLLQGPVFIKEPSNSIFPVGSEDKKITLHCEARGNPSPHYRWQLNGSDIDMSMEHRYKLNGGNLVVINPNRNWDTGTYQCFATNSLGTIVSREAKLQFAYLENFKTKMRSTVSVREGQGVVLLCGPPPHSGELSYAWIFNEYPSFVEEDSRRFVSQETGHLYISKVEPSDVGNYTCVVTSMVTNARVLGSPTPLVLRSDGVMGEYEPKIEVQFPETLPAAKGSTVKLECFALGNPIPQINWRRSDGLPFSSKIKLRKFSGVLEIPNFQQEDAGSYECIAENSRGKNVARGRLTYYAKPHWVQLIKDVEIAVEDSLYWECRASGKPKPSYRWLKNGAALVLEERTQIENGALTISNLSVTDSGMFQCIAENKHGLVYSSAELKVVASAPDFSKNPMKKLVQVQVGSLVSLDCKPRASPRALSSWKKGDVSVQEHERISLLNDGGLKIANVTKADAGTYTCMAENQFGKANGTTHLVVTEPTRITLAPSNMDVSVGESVILPCQVQHDPLLDIIFTWYFNGALADFKKDGSHFEKVGGSSSGDLMIRNIQLKHSGKYVCMVQTGVDSVSSAADLIVRGSPGPPENVKVDEITDTTAQLSWKEGKDNHSPVISYSIQARTPFSVGWQTVTTVPEVIDGKTHTATVVELNPWVEYEFRVVASNKIGGGEPSLPSEKVRTEEAVPEVPPSEVNGGGGSRSELVITWDPVPEELQNGEGFGYVVAFRPLGVTTWIQTVVTSPDTPRYVFRNESIVPYSPYEVKVGVYNNKGEGPFSPVTTVFSAEEEPTVAPSQVSANSLSSSEIEVSWNTIPWKLSNGHLLGYEVRYWNGGGKEESSSKMKVAGNETSARLRGLKSNLAYYTAVRAYNSAGAGPFSATVNVTTKKTPPSQPPGNVVWNATDTKVLLNWEQVKAMENESEVTGYKVFYRTSSQNNVQVLNTNKTSAELVLPIKEDYIIEVKATTDGGDGTSSEQIRIPRITSMDARGSTSAISNVHPMSSYMPIVLFLIVYVLW

SEQ ID NO: 15 - protein, 1009 aa, Homo sapiens
ELLLQGPVFIKEPSNSIFPVGSEDKKITLHCEARGNPSPHYRWQLNGSDIDMSMEHRYKLNGGNLVVINPNRNWDTGTYQCFATNSLGTIVSREAKLQFAYLENFKTKMRSTVSVREGQGVVLLCGPPPHSGELSYAWIFNEYPSFVEEDSRRFVSQETGHLYISKVEPSDVGNYTCVVTSMVTNARVLGSPTPLVLRSDGVMGEYEPKIEVQFPETLPAAKGSTVKLECFALGNPIPQINWRRSDGLPFSSKIKLRKFSGVLEIPNFQQEDAGSYECIAENSRGKNVARGRLTYYAKPHWVQLIKDVEIAVEDSLYWECRASGKPKPSYRWLKNGAALVLEERTQIENGALTISNLSVTDSGMFQCIAENKHGLVYSSAELKVVASAPDFSKNPMKKLVQVQVGSLVSLDCKPRASPRALSSWKKGDVSVQEHERISLLNDGGLKIANVTKADAGTYTCMAENQFGKANGTTHLVVTEPTRITLAPSNMDVSVGESVILPCQVQHDPLLDIIFTWYFNGALADFKKDGSHFEKVGGSSSGDLMIRNIQLKHSGKYVCMVQTGVDSVSSAADLIVRGSPGPPENVKVDEITDTTAQLSWKEGKDNHSPVISYSIQARTPFSVGWQTVTTVPEVIDGKTHTATVVELNPWVEYEFRVVASNKIGGGEPSLPSEKVRTEEAVPEVPPSEVNGGGGSRSELVITWDPVPEELQNGEGFGYVVAFRPLGVTTWIQTVVTSPDTPRYVFRNESIVPYSPYEVKVGVYNNKGEGPFSPVTTVFSAEEEPTVAPSQVSANSLSSSEIEVSWNTIPWKLSNGHLLGYEVRYWNGGGKEESSSKMKVAGNETSARLRGLKSNLAYYTAVRAYNSAGAGPFSATVNVTTKKTPPSQPPGNVVWNATDTKVLLNWEQVKAMENESEVTGYKVFYRTSSQNNVQVLNTNKTSAELVLPIKEDYIIEVKATTDGGDGTSSEQIRIPRITSMDARGSTSAISNVHPMSSYMPIVLFLIVYVLW

SEQ ID NO: 16 - RNA, 1047 nt, Homo sapiens
  source 1..1047: mol_type = mRNA
atgagtgacctgggagctgtcatttccctcctgctctggggacgacagctttttgcactgtactcaggcaatgatgtcacggatatttcagatgaccgcttcccgaagccccctgagattgcaaatggctatgtggagcacttgtttcgctaccagtgtaagaactactacagactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccagcactgggttcagaagaccatagctgagaactaa

SEQ ID NO: 17 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 18 - RNA, 1338 nt, Homo sapiens
  source 1..1338: mol_type = mRNA
atggctgagttaaagtacatttctggatttgggaatgagtgttcttcagaggatcctcgctgcccaggttccctgccagaaggacagaataatcctcaggtctgcccctacaatctctatgctgagcagctctcaggatcggctttcacttgtccacggagcaccaataagagaagctggctgtataggattctaccttcagtttctcacaagccctttgaatccattgacgaaggccaagtcactcacaactgggatgaagttgatcctgatcctaaccagcttagatggaaaccatttgagattccaaaagcatctcagaagaaagtagactttgtgagtggcctgcataccttgtgtggagctggagacataaagtctaacaatgggcttgctatccacattttcctctgcaatacctccatggagaacagatgcttttacaattcagatggggacttcttgattgttccgcagaaagggaaccttctcatttacaccgagtttggcaagatgcttgtacagcccaatgagatctgcgtcattcagagaggaatgcggttcagcatagatgtctttgaggagaccaggggctacatcttggaggtctatggtgtccactttgagttacctgaccttggaccaattggggccaatggcttggccaatcctcgtgatttcttgatacccattgcctggtatgaggatcgccaagtaccaggtggttacacggtcattaataaataccagggcaagctgtttgctgccaaacaggatgtctccccgttcaatgttgtggcctggcacgggaattatacaccctacaagtacaacctgaagaatttcatggttatcaactcagtggcctttgaccatgcagacccatccattttcacagtattgactgctaagtctgtccgccctggagtggccattgctgattttgtcatcttcccacctcgatggggggttgctgataagaccttcaggcctccttattaccataggaactgcatgagtgagttcatgggactcatccgaggtcactatgaggcaaagcaaggtgggttcctgccagggggagggagtctacacagcacaatgaccccccatggacctgatgctgactgctttgagaaggccagcaaggtcaagctggcacctgagaggattgccgatggcaccatggcatttatgtttgaatcatctttaagtctggcggtcacaaagtggggactcaaggcctccaggtgtttggatgagaactaccacaagtgctgggagccactcaagagccacttcactcccaactccaggaacccagcagaacctaattga

SEQ ID NO: 19 - protein, 445 aa, Homo sapiens
MAELKYISGFGNECSSEDPRCPGSLPEGQNNPQVCPYNLYAEQLSGSAFTCPRSTNKRSWLYRILPSVSHKPFESIDEGQVTHNWDEVDPDPNQLRWKPFEIPKASQKKVDFVSGLHTLCGAGDIKSNNGLAIHIFLCNTSMENRCFYNSDGDFLIVPQKGNLLIYTEFGKMLVQPNEICVIQRGMRFSIDVFEETRGYILEVYGVHFELPDLGPIGANGLANPRDFLIPIAWYEDRQVPGGYTVINKYQGKLFAAKQDVSPFNVVAWHGNYTPYKYNLKNFMVINSVAFDHADPSIFTVLTAKSVRPGVAIADFVIFPPRWGVADKTFRPPYYHRNCMSEFMGLIRGHYEAKQGGFLPGGGSLHSTMTPHGPDADCFEKASKVKLAPERIADGTMAFMFESSLSLAVTKWGLKASRCLDENYHKCWEPLKSHFTPNSRNPAEPN

SEQ ID NO: 20 - RNA, 531 nt, Homo sapiens
  source 1..531: mol_type = mRNA
atggcgcgggtgctgaaggctgcagccgcgaatgccgtagggcttttttccagacttcaagctcccattccaacagtaagagcttcttccacatcacagcccttggatcaagtgacaggttctgtgtggaacctgggtcgactcaaccatgtagccatagcagtgccagatttggaaaaggctgcagcattttataagaatattctgggggcccaggtaagtgaagcggtccctcttcctgaacatggagtatctgttgtttttgtcaacctgggaaataccaagatggaactgcttcatccattgggacgtgacagtccaattgcaggttttctgcagaaaaacaaggctggaggaatgcatcacatctgcatcgaggtggataatattaatgcagctgtgatggatttgaaaaaaaagaagatccgcagtctaagtgaagaggtcaaaataggagcacatggaaaaccagtgatttttctccatcctaaagactgtggtggagtccttgtggaactggagcaagcttga

SEQ ID NO: 21 - protein, 176 aa, Homo sapiens
MARVLKAAAANAVGLFSRLQAPIPTVRASSTSQPLDQVTGSVWNLGRLNHVAIAVPDLEKAAAFYKNILGAQVSEAVPLPEHGVSVVFVNLGNTKMELLHPLGRDSPIAGFLQKNKAGGMHHICIEVDNINAAVMDLKKKKIRSLSEEVKIGAHGKPVIFLHPKDCGGVLVELEQA

SEQ ID NO: 22 - protein, 140 aa, Homo sapiens
QVTGSVWNLGRLNHVAIAVPDLEKAAAFYKNILGAQVSEAVPLPEHGVSVVFVNLGNTKMELLHPLGRDSPIAGFLQKNKAGGMHHICIEVDNINAAVMDLKKKKIRSLSEEVKIGAHGKPVIFLHPKDCGGVLVELEQA

SEQ ID NO: 23 - RNA, 1392 nt, Homo sapiens
  source 1..1392: mol_type = mRNA
atgtctgaagaagaggcggctcagatccccagatccagtgtgtgggagcaggaccagcagaacgtggtgcagcgtgtggtggctctgcccctggtcagggccacgtgcaccgcggtctgcgatgtttacagtgcagccaaggacaggcacccgctgctgggctccgcctgccgcctggctgagaactgcgtgtgcggcctgaccacccgtgccctggaccacgcccagccgctgctcgagcacctgcagccccagctggccactatgaacagcctcgcctgcaggggcctggacaagctggaagagaagcttccctttctccagcaaccttcggagacggtggtgacctcagccaaggacgtggtggccagcagtgtcacgggtgtggtggacctggcccggaggggccggcgctggagcgtggagctgaagcgctccgtgagccatgctgtggatgttgtactggaaaaatcagaggagctggtggatcacttcctgcccatgacggaggaagagctcgcggcactggcggctgaggctgaaggccctgaagtgggttcggtggaggatcagaggagacagcagggctactttgtgcgcctcggctccctgtcagcacggatccgccacctggcctacgagcactctgtggggaaactgaggcagagcaaacaccgtgcccaggacaccctggcccagctgcaggagacgctggagctgatagaccacatgcagtgtggggtgacccccaccgccccggcctgccctgggaaggtgcacgagctgtggggggaatggggccagcgccctccggagagccgccgccggagccaggcagagctggagacgctggtgctgtcccgcagcctgacccaggagctgcagggcacggtagaggctctggagtccagcgtgcggggcctgcccgccggcgcccaggagaaggtggctgaggtgcggcgcagtgtggatgccctgcagaccgccttcgctgatgcccgctgcttcagggacgtgccagcggccgcgctggccgagggccggggtcgcgtggcccacgcgcacgcctgcgtggacgagctgctggagctggtggtgcaggccgtgccgctgccctggctggtgggacccttcgcgcccatccttgtggagcgacccgagcccctgcccgacctggcggacctggtggacgaggtcatcgggggccctgacccccgctgggcgcacctggactggccggcccagcagagagcctgggaggcagagcacagggacgggagtgggaatggggatggggacaggatgggtgttgccggggacatctgcgagcaggaacccgagacccccagctgcccggtcaagcacaccctgatgcccgagctggacttctga

SEQ ID NO: 24 - protein, 463 aa, Homo sapiens
MSEEEAAQIPRSSVWEQDQQNVVQRVVALPLVRATCTAVCDVYSAAKDRHPLLGSACRLAENCVCGLTTRALDHAQPLLEHLQPQLATMNSLACRGLDKLEEKLPFLQQPSETVVTSAKDVVASSVTGVVDLARRGRRWSVELKRSVSHAVDVVLEKSEELVDHFLPMTEEELAALAAEAEGPEVGSVEDQRRQQGYFVRLGSLSARIRHLAYEHSVGKLRQSKHRAQDTLAQLQETLELIDHMQCGVTPTAPACPGKVHELWGEWGQRPPESRRRSQAELETLVLSRSLTQELQGTVEALESSVRGLPAGAQEKVAEVRRSVDALQTAFADARCFRDVPAAALAEGRGRVAHAHACVDELLELVVQAVPLPWLVGPFAPILVERPEPLPDLADLVDEVIGGPDPRWAHLDWPAQQRAWEAEHRDGSGNGDGDRMGVAGDICEQEPETPSCPVKHTLMPELDF

SEQ ID NO: 25 - RNA, 2943 nt, Homo sapiens
  source 1..2943: mol_type = mRNA
atggcgctggctgtcttgcgggtcctggagccctttccgaccgagacacccccgttggcagtgctgctgccacccgggggcccgtggccggcggcggagctgggcctggtgctggccctgaggcctgcaggggagagcccggcagggccggcgctgctggtggcagccctggaggggccggacgcgggcaccgaagagcagggtcccgggccgccgcagctactggttagccgcgcgctgctgcggctcctggcactgggctccggggcctgggtgcgggcgcgggcggtgcggcggcccccggcgctaggttgggcactgcttggcacctcgctggggcctgggctcggaccgcgagtcgggccgctgctggtgaggcgcggagagaccctcccagtgcccggaccgcgggtgctggagacgcggccggcgttgcaagggctgctgggcccagggactcggctggctgtgactgagctccgcgggcgggccagactgtgtccagagtctggggacagcagtcggcccccacccccgcccgtggtgtcctcctttgcggtttctggcacagtgcggcgactccagggagttctgggagggactggagattcactaggggtgagccggagctgtctccgtggccttggcctcttccagggcgaatgggtgtgggtggcccaggccagagagtcatcgaacacttcacagccgcacttggctagggtgcaggtcctagaacctcgctgggacctctctgatagactgggacccggctctggaccgctgggagagcccctcgctgacggactggcgcttgtccctgccactttggcttttaatcttggctgtgaccccctggaaatgggagagctcagaattcagaggtacttggaaggctccatcgcccctgaagacaaaggaagctgctcattgctgcctgggcctccatttgccagagagttacacatcgaaattgtgtcttctccccactacagcactaatggaaattatgacggtgttctttaccggcactttcagatacccagggtagtccaggaaggggatgttctatgtgtgccaacaattgggcaagtagagatcctggaaggaagtccagagaaactgcccaggtggcgggaaatgttttttaaagtgaagaaaacagttggggaagctccagatggaccagccagtgcctacttggccgacaccacccatacctccttgtacatggtgggttctaccctgagccctgttccatggctcccttcagaggaatccactctctggagcagtttgtctcctccaggcctggaggccttggtgtctgaactctgtgctgtcctgaagcctcgcctccagccagggggtgccctgctgacaggaactagcagtgtccttctacggggccccccaggctgtgggaagaccacagtagttgctgctgcctgtagtcaccttgggctccacttactgaaggtgccctgctccagcctctgtgcagaaagtagtggggctgtggagacaaaactgcaggccatcttctcccgggcccgccgttgccggcctgcagtcctgttgctcacagctgtggaccttctgggccgggaccgtgatgggctgggtgaggatgcccgtgtgatggctgtgctgcgtcacctcctcctcaatgaggaccccctcaacagctgccctcccctcatggttgtggccaccacaagccgggcccaggacctgcctgctgatgtgcagacagcatttcctcatgagctcgaggtgcctgctctgtcagaggggcagcggctcagcatcctgcgggccctcactgcccaccttcccctgggccaggaggtgaacttggcacagctagcacggcggtgtgcaggctttgtggtaggggatctctatgcccttctgacccacagcagccgggcagcctgcaccaggatcaagaactcaggtttggcaggtggcttgactgaggaggatgagggggagctgtgtgctgccggctttcctctcctggctgaggactttgggcaggcactggagcaactgcagacagctcactcccaggccgttggagcccccaagatcccctcagtgtcctggcatgatgtgggtgggctgcaggaggtgaagaaggagatcctggagaccattcagctccccctggagcaccctgagctactgagcctgggcctgagacgctcaggccttctgctccatgggccccctggcaccggcaagacccttctggccaaggcagtagccactgagtgcagccttaccttcctcagcgtgaaggggccagagctcattaacatgtatgtgggccaaagtgaggagaatgtgcgggaagtgtttgccagggccagggctgcagctccatgcattatcttctttgatgaactggactctttggccccaagccgggggcgaagtggagattctggaggagtgatggacagggtggtgtctcagctccttgccgagctagatgggctgcacagcactcaggatgtgtttgtgattggagccaccaacagaccagatctcctggaccctgcccttctgcggcctggcagatttgacaagctggtgtttgtgggggcaaatgaggaccgggcctcccagctacgcgttctaagtgccatcacacgcaaattcaagctagagccatctgtgagcctggtaaacgtgctagattgctgccctccccagctgacgggcgcggacctctactctctctgctctgatgctatgacagctgccctcaaacgcagggttcatgacctggaggaagggctggagccaggtagctcagcactgatgctcaccatggaggacttgctgcaggctgccgcccggctgcaaccctcagtcagtgagcaggagctgctccggtacaagcgcatccagcgcaagtttgctgcctgctag

SEQ ID NO: 26 - protein, 980 aa, Homo sapiens
MALAVLRVLEPFPTETPPLAVLLPPGGPWPAAELGLVLALRPAGESPAGPALLVAALEGPDAGTEEQGPGPPQLLVSRALLRLLALGSGAWVRARAVRRPPALGWALLGTSLGPGLGPRVGPLLVRRGETLPVPGPRVLETRPALQGLLGPGTRLAVTELRGRARLCPESGDSSRPPPPPVVSSFAVSGTVRRLQGVLGGTGDSLGVSRSCLRGLGLFQGEWVWVAQARESSNTSQPHLARVQVLEPRWDLSDRLGPGSGPLGEPLADGLALVPATLAFNLGCDPLEMGELRIQRYLEGSIAPEDKGSCSLLPGPPFARELHIEIVSSPHYSTNGNYDGVLYRHFQIPRVVQEGDVLCVPTIGQVEILEGSPEKLPRWREMFFKVKKTVGEAPDGPASAYLADTTHTSLYMVGSTLSPVPWLPSEESTLWSSLSPPGLEALVSELCAVLKPRLQPGGALLTGTSSVLLRGPPGCGKTTVVAAACSHLGLHLLKVPCSSLCAESSGAVETKLQAIFSRARRCRPAVLLLTAVDLLGRDRDGLGEDARVMAVLRHLLLNEDPLNSCPPLMVVATTSRAQDLPADVQTAFPHELEVPALSEGQRLSILRALTAHLPLGQEVNLAQLARRCAGFVVGDLYALLTHSSRAACTRIKNSGLAGGLTEEDEGELCAAGFPLLAEDFGQALEQLQTAHSQAVGAPKIPSVSWHDVGGLQEVKKEILETIQLPLEHPELLSLGLRRSGLLLHGPPGTGKTLLAKAVATECSLTFLSVKGPELINMYVGQSEENVREVFARARAAAPCIIFFDELDSLAPSRGRSGDSGGVMDRVVSQLLAELDGLHSTQDVFVIGATNRPDLLDPALLRPGRFDKLVFVGANEDRASQLRVLSAITRKFKLEPSVSLVNVLDCCPPQLTGADLYSLCSDAMTAALKRRVHDLEEGLEPGSSALMLTMEDLLQAAARLQPSVSEQELLRYKRIQRKFAAC

SEQ ID NO: 27 - RNA, 534 nt, Homo sapiens
  source 1..534: mol_type = mRNA
atgggggtctcggtggatgtgcaccaggtgtacaagtaccccttcgagcaggtggtcgccagctttctccgaaagtaccccaaccccatggataaaaatgtcatctcagtaaaaatcatggaggaaaaaagagatgaatcaacaggggtcatctacagaaagaggattgcaatctgtcagaacgtggttccagaaattttaaggaaggtgagcattttgaaagtacctaatatccaattagaagaggagtcatggctcaatcctcgggaaagaaacatggccatacggagtcactgccttacgtggacacagtatgcatccatgaaggaagagtctgtcttccgggaaagtatggaaaacccaaattggacagagttcattcaaagaggcaggatttcaatcacaggggttggatttctcaactgtgttttagaaacttttgccagcacattcttacgacagggagcccagaagggaattagaatcatggagatgctgctaaaggaacagtgtggtgcccccttagctgaataa

SEQ ID NO: 28 - protein, 177 aa, Homo sapiens
MGVSVDVHQVYKYPFEQVVASFLRKYPNPMDKNVISVKIMEEKRDESTGVIYRKRIAICQNVVPEILRKVSILKVPNIQLEEESWLNPRERNMAIRSHCLTWTQYASMKEESVFRESMENPNWTEFIQRGRISITGVGFLNCVLETFASTFLRQGAQKGIRIMEMLLKEQCGAPLAE

SEQ ID NO: 29 - RNA, 651 nt, Homo sapiens
  source 1..651: mol_type = mRNA
atgggcacccgcgacgacgagtacgactacctctttaaagttgtccttattggagattctggtgttggaaagagtaatctcctgtctcgatttactcgaaatgagtttaatctggaaagcaagagcaccattggagtagagtttgcaacaagaagcatccaggttgatggaaaaacaataaaggcacagatatgggacacagcagggcaagagcgatatcgagctataacatcagcatattatcgtggagctgtaggtgccttattggtttatgacattgctaaacatctcacatatgaaaatgtagagcgatggctgaaagaactgagagatcatgctgatagtaacattgttatcatgcttgtgggcaataagagtgatctacgtcatctcagggcagttcctacagatgaagcaagagcttttgcagaaaagaatggtttgtcattcattgaaacttcggccctagactctacaaatgtagaagctgcttttcagacaattttaacagagatttaccgcattgtttctcagaagcaaatgtcagacagacgcgaaaatgacatgtctccaagcaacaatgtggttcctattcatgttccaccaaccactgaaaacaagccaaaggtgcagtgctgtcagaacatctaa

SEQ ID NO: 30 - protein, 216 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 31 - RNA, 618 nt, Homo sapiens
  source 1..618: mol_type = mRNA
atgtcaaacttcttgcatctcaaatataatgagaaatctgtttctgttacaaaagcccttacagtgaggtttcttactaagcgattcattggagaatatgcttctaattttgaatctatctataagaagcacttgtgtttggaaaggaaacaactaaatctagaaatatatgacccttgttctcaaacacagaaagcaaaattctccctcacaagtgagcttcactgggcagatgggtttgttattgtgtatgacatcagtgataggtcttcatttgcttttgcaaaagcgctgatctacagaatccgggagccacaaactagtcattgtaaaagagctgtggaatcagcagtgtttttggttggcaacaaacgagatctttgtcatgtgcgagaggttggctgggaagaagggcaaaagctggcactggaaaaccgatgccaattctgtgaactgtctgcagcagagcagtctctggaggtggaaatgatgtttatcagaattatcaaggacatcctgataaacttcaaactcaaagaaaagagacgtcccagtggatctaaatcaatggccaaattgatcaataatgtatttggaaagagaaggaaatctgtttag

SEQ ID NO: 32 - protein, 205 aa, Homo sapiens
MSNFLHLKYNEKSVSVTKALTVRFLTKRFIGEYASNFESIYKKHLCLERKQLNLEIYDPCSQTQKAKFSLTSELHWADGFVIVYDISDRSSFAFAKALIYRIREPQTSHCKRAVESAVFLVGNKRDLCHVREVGWEEGQKLALENRCQFCELSAAEQSLEVEMMFIRIIKDILINFKLKEKRRPSGSKSMAKLINNVFGKRRKSV

SEQ ID NO: 33 - RNA, 3318 nt, Homo sapiens
  source 1..3318: mol_type = mRNA
atgctcgcctactgcgtgcaggatgccaccgtggtggacgtggagaagcggaggaacccctccaagcactacgtatacataatcaatgtgacctggtctgactccacctcccagactatctaccggaggtacagcaagttctttgacctgcagatgcagcttttggataagtttcccattgaaggtggccagaaggaccccaagcaaaggatcatccccttcctcccaggcaagatcctcttccgcagaagccacatccgggacgtagctgtgaagagactgaagcccatcgatgaatactgccgggcacttgtccggctgcccccccacatctcacagtgtgacgaagtcttccggttcttcgaggctcgacccgaggatgtcaaccctccaaaagaggactatggcagttccaagaggaaatcagtgtggctgtccagctgggctgagtcgcccaagaaggacgtgacaggtgccgacgccaccgccgagcccatgatcctggaacagtacgtggtggtgtccaactataagaagcaggagaactcggagctgagcctccaggccggggaggtggtggatgtcatcgagaagaacgagagcggctggtggttcgtgagcacttctgaggagcagggctgggtccctgccacctacctggaggcccagaatggtactcgggatgactccgacatcaacacctctaagactggagaagaggagaagtatgtcaccgtgcagccttacaccagccaaagcaaggacgagattggctttgagaagggcgtcacagtggaggtgatccggaagaatctggaaggctggtggtatatcagatacctgggcaaagagggctgggcgccagcatcctacctgaagaaggccaaggatgacctgccaacccggaagaagaacctggccggcccagtggagatcattgggaacatcatggagatcagcaacctgctgaacaagaaggcgtctggggacaaggaaactccaccagccgaaggcgagggccatgaggcccccattgccaagaaggagatcagcctgcccatcctctgcaatgcctccaatggcagtgccgtgggcgttcctgacaggactgtctccaggctggcccagggctctccagctgtggccaggattgcccctcagcgggcccagatcagctccccgaacctacggacaagacctccaccacgcagagaatccagcctggggttccaactgccaaagccaccagagcccccttctgttgaggtggagtactacaccattgccgaattccagtcgtgcatttccgatggcatcagctttcggggtggacagaaggcagaggtcattgataagaactcaggtggctggtggtacgtgcagatcggtgagaaggagggctgggcccccgcatcatacatcgataagcgcaagaagcccaacctgagccgccgcacaagcacgctgacccggcccaaggtgcccccgccagcaccccccagcaagcccaaggaggccgaggagggccctacgggggccagtgagagccaggactccccgcggaagctcaagtatgaggagcctgagtatgacatccctgcattcggctttgactcagagcctgagctgagcgaggagcccgtggaggacagagcctcaggggagaggcggcctgcccagccccaccggccctcgccggcctcttctctgcagcgggcccgcttcaaggtgggtgagtcttcagaggatgtggccctggaagaggagaccatctatgagaatgagggcttccggccatatgcagaggacaccctgtcagccagaggctcctccggggacagcgactccccaggcagctcctcgctgtccctgaccaggaaaaactcccccaaatcaggctcccccaagtcatcatcactcctaaagctcaaggcagagaagaatgcccaggcagaaatggggaagaaccactcctcagcctccttttcctcatccatcaccatcaacaccacttgctgctcctcctcttcctcctcctcctcttccttgtccaaaaccagtggcgacctgaagccccgctctgcttcggacgcaggcatccgcggcactcccaaggtcagggcaaagaaggatgctgatgcgaacgctgggctgacctcctgtccccgggccaagccatcggtccggcccaagccattcctaaaccgagcagagtcgcagagccaagagaagatggacatcagcactttacggcgccagctgagacccacaggccagctccgtggagggctcaagggctccaagagtgaggattcggagctgcccccgcagacggcctccgaggctcccagtgaggggtctaggagaagctcatccgacctcatcaccctcccagccaccactcccccatgtcccaccaagaaggaatgggaagggccagccacctcgtacatgacatgcagcgcctaccagaaggtccaggactcggagatcagcttccccgcgggcgtggaggtgcaggtgctggagaagcaggagagcgggtggtggtatgtgaggtttggggagctggagggctgggccccttcccactatttggtgctggatgagaacgagcaacctgacccctctggcaaagagctggacacagtgcccgccaagggcaggcagaacgaaggcaaatcagacagcctggagaagatcgagaggcgcgtccaagcactgaacaccgtcaaccagagcaagaaggccacgccccccatcccctccaaacctcccgggggcttcggcaagacctcaggcactccagcggtgaagatgaggaacggagtgcggcaggtggcggtcaggccccagtcggtgtttgtgtccccgccacccaaggacaacaacctgtcctgcgccctgcggaggaatgagtcactcacggccactgatggcctccgaggcgtccgacggaactcctcctttagcactgctcgctccgctgccgccgaggccaagggccgcctggccgaacgggctgccagccagggttcagactcacccctactgcccgcccagcgcaacagcatacccgtgtcccctgtgcgccccaagcccatcgagaagtctcagttcatccacaataacctcaaagatgtgtacgtctctatcgcagactacgagggggatgaggagacagcaggcttccaggagggggtgtccatggaggttctggagaggaaccctaatggctggtggtactgccagatcctggatggtgtgaagcccttcaaaggctgggtgccttccaactaccttgagaaaaagaactag

SEQ ID NO: 34 - protein, 1105 aa, Homo sapiens
MLAYCVQDATVVDVEKRRNPSKHYVYIINVTWSDSTSQTIYRRYSKFFDLQMQLLDKFPIEGGQKDPKQRIIPFLPGKILFRRSHIRDVAVKRLKPIDEYCRALVRLPPHISQCDEVFRFFEARPEDVNPPKEDYGSSKRKSVWLSSWAESPKKDVTGADATAEPMILEQYVVVSNYKKQENSELSLQAGEVVDVIEKNESGWWFVSTSEEQGWVPATYLEAQNGTRDDSDINTSKTGEEEKYVTVQPYTSQSKDEIGFEKGVTVEVIRKNLEGWWYIRYLGKEGWAPASYLKKAKDDLPTRKKNLAGPVEIIGNIMEISNLLNKKASGDKETPPAEGEGHEAPIAKKEISLPILCNASNGSAVGVPDRTVSRLAQGSPAVARIAPQRAQISSPNLRTRPPPRRESSLGFQLPKPPEPPSVEVEYYTIAEFQSCISDGISFRGGQKAEVIDKNSGGWWYVQIGEKEGWAPASYIDKRKKPNLSRRTSTLTRPKVPPPAPPSKPKEAEEGPTGASESQDSPRKLKYEEPEYDIPAFGFDSEPELSEEPVEDRASGERRPAQPHRPSPASSLQRARFKVGESSEDVALEEETIYENEGFRPYAEDTLSARGSSGDSDSPGSSSLSLTRKNSPKSGSPKSSSLLKLKAEKNAQAEMGKNHSSASFSSSITINTTCCSSSSSSSSSLSKTSGDLKPRSASDAGIRGTPKVRAKKDADANAGLTSCPRAKPSVRPKPFLNRAESQSQEKMDISTLRRQLRPTGQLRGGLKGSKSEDSELPPQTASEAPSEGSRRSSSDLITLPATTPPCPTKKEWEGPATSYMTCSAYQKVQDSEISFPAGVEVQVLEKQESGWWYVRFGELEGWAPSHYLVLDENEQPDPSGKELDTVPAKGRQNEGKSDSLEKIERRVQALNTVNQSKKATPPIPSKPPGGFGKTSGTPAVKMRNGVRQVAVRPQSVFVSPPPKDNNLSCALRRNESLTATDGLRGVRRNSSFSTARSAAAEAKGRLAERAASQGSDSPLLPAQRNSIPVSPVRPKPIEKSQFIHNNLKDVYVSIADYEGDEETAGFQEGVSMEVLERNPNGWWYCQILDGVKPFKGWVPSNYLEKKN

SEQ ID NO: 35 - RNA, 1548 nt, Homo sapiens
  source 1..1548: mol_type = mRNA
atggtgctctcccaggaggagccggactccgcgcggggcacgagcgaggcgcagccgctcggccccgcgcccacgggggccgctccgccgcccggcccgggaccctcggacagccccgaggcggctgtcgagaaggtggaggtggagctggcggggccggcgaccgcggagccccatgagccccccgaaccccccgagggcggctggggctggctggtgatgctggcggccatgtggtgcaacgggtcggtgttcggcatccagaacgcttgcggggtgctcttcgtgtccatgctggaaaccttcggctccaaagacgatgacaagatggtctttaagacagcatgggtaggttctctctccatggggatgattttcttttgctgcccaatagtcagcgtcttcacagacctatttggttgtcggaaaacagctgtcgtgggtgctgctgttggatttgttgggctcatgtccagttcttttgtaagttccatcgagcctctgtaccttacctatggaatcatatttgcctgcggctgctcctttgcataccagccttcattggtcattttgggacactatttcaagaagcgccttggactggtgaatggcattgtcactgctggcagcagtgtcttcacaatcctgctgcctttgctcttaagggttctgattgacagcgtgggcctcttttacacattgagggtgctctgcatcttcatgtttgttctctttctggctggctttacttaccgacctcttgctaccagtaccaaagataaagagagtggaggtagcggatcctccctcttttccaggaaaaagttcagtcctccaaaaaaaattttcaattttgccatcttcaaggtgacagcttatgcagtgtgggcagttggaataccacttgcactttttggatactttgtgccttatgttcacttgatgaaacatgtaaatgaaagatttcaagatgaaaaaaataaagaggttgttctcatgtgcattggcgtcacttcaggagttggacgactgctctttggccggattgcagattatgtgcctggtgtgaagaaggtttatctacaggtactctcctttttcttcattggtctgatgtccatgatgattcctctgtgtagcatctttggggccctcattgctgtgtgcctcatcatgggtctcttcgatggatgcttcatttccattatggctcccatagcctttgagttagttggtgcccaggatgtctcccaagcaattggatttctgctcggattcatgtctatacccatgactgttggcccacccattgcagggttacttcgtgacaaactgggctcctatgatgtggcattctacctcgctggagtccctccccttattggaggtgctgtgctttgttttatcccgtggatccatagtaagaagcaaagagagatcagtaaaaccactggaaaagaaaagatggagaaaatgttggaaaaccagaactctctgctgtcaagttcatctggaatgttcaagaaagaatctgactctattatttaa

SEQ ID NO: 36 - protein, 515 aa, Homo sapiens
MVLSQEEPDSARGTSEAQPLGPAPTGAAPPPGPGPSDSPEAAVEKVEVELAGPATAEPHEPPEPPEGGWGWLVMLAAMWCNGSVFGIQNACGVLFVSMLETFGSKDDDKMVFKTAWVGSLSMGMIFFCCPIVSVFTDLFGCRKTAVVGAAVGFVGLMSSSFVSSIEPLYLTYGIIFACGCSFAYQPSLVILGHYFKKRLGLVNGIVTAGSSVFTILLPLLLRVLIDSVGLFYTLRVLCIFMFVLFLAGFTYRPLATSTKDKESGGSGSSLFSRKKFSPPKKIFNFAIFKVTAYAVWAVGIPLALFGYFVPYVHLMKHVNERFQDEKNKEVVLMCIGVTSGVGRLLFGRIADYVPGVKKVYLQVLSFFFIGLMSMMIPLCSIFGALIAVCLIMGLFDGCFISIMAPIAFELVGAQDVSQAIGFLLGFMSIPMTVGPPIAGLLRDKLGSYDVAFYLAGVPPLIGGAVLCFIPWIHSKKQREISKTTGKEKMEKMLENQNSLLSSSSGMFKKESDSII

SEQ ID NO: 37 - RNA, 1644 nt, Homo sapiens
  source 1..1644: mol_type = mRNA
atggcctttcaggacctcctagatcaagttggaggcctggggagattccagatccttcagatggttttccttataatgttcaacgtcatagtataccatcaaactcagctggagaacttcgcagcattcatacttgatcatcgctgctgggttcatatactggacaatgacactatccctgacaatgaccctgggaccctcagccaggatgccctcctgagaatctccatcccattcgactcaaatctgaggccagagaagtgtcgtcgctttgtccatccccagtggaagctcattcatctgaatgggaccttccccaacacgagtgagccagatacagagccctgtgtggatggctgggtatatgaccaaagctccttcccttccaccattgtgactaagtgggatctggtatgcgaatctcaaccactgaattcagtagctaaatttctattcatggctggaatgatggtgggaggcaacctatatggccatttgtcagacaggtttgggagaaagttcgtgctcagatggtcttacctccagctcgccattgtaggcacctgtgcggcctttgctcccaccatcctcgtatactgctccctgcgcttcttggctggggctgctacatttagcatcattgtaaatactgttttgttaattgtagagtggataactcaccaattctgtgccatggcattgacattgacactttgtgctgctagtattggacatataaccctgggaagcctggcttttgtcattcgagaccagtgcatcctccagttggtgatgtctgcaccatgctttgtcttctttctgttctcaaggtggctggcagagtctgctcggtggctcattatcaacaacaaaccagaagagggcttaaaggaacttagaaaagctgcacacaggaatggaatgaagaatgctgaagacatcctaaccatggaggttttgaaatccaccatgaagcaagaactggaggcagcacagaaaaagcattctctttgtgaattgctccgcatacccaacatatgtaaaagaatctgtttcctgtcctttgtgagatttgcaagtaccatccctttttggggccttactttgcacctccagcatctgggaaacaatgttttcctgttgcagactctctttggtgcagtcaccctcctggccaattgtgttgcaccttgggcactgaatcacatgagccgtcgactaagccagatgcttctcatgttcctactggcaacctgccttctggccatcatatttgtgcctcaagaaatgcagaccctgcgtgtggttttggcaaccctgggtgtgggagctgcttctcttggcattacctgttctactgcccaagaaaatgaactaattccttccataatcaggggaagagctactggaatcactggaaactttgctaatattgggggagccctggcttccctcatgatgatcctaagcatatattctcgacccctgccctggatcatctatggagtctttgccatcctctctggccttgttgtcctcctccttcctgaaaccaggaaccagcctcttcttgacagcatccaggatgtggaaaatgagggagtaaatagcctagctgcccctcagaggagctctgtgctatag

SEQ ID NO: 38 - protein, 547 aa, Homo sapiens
MAFQDLLDQVGGLGRFQILQMVFLIMFNVIVYHQTQLENFAAFILDHRCWVHILDNDTIPDNDPGTLSQDALLRISIPFDSNLRPEKCRRFVHPQWKLIHLNGTFPNTSEPDTEPCVDGWVYDQSSFPSTIVTKWDLVCESQPLNSVAKFLFMAGMMVGGNLYGHLSDRFGRKFVLRWSYLQLAIVGTCAAFAPTILVYCSLRFLAGAATFSIIVNTVLLIVEWITHQFCAMALTLTLCAASIGHITLGSLAFVIRDQCILQLVMSAPCFVFFLFSRWLAESARWLIINNKPEEGLKELRKAAHRNGMKNAEDILTMEVLKSTMKQELEAAQKKHSLCELLRIPNICKRICFLSFVRFASTIPFWGLTLHLQHLGNNVFLLQTLFGAVTLLANCVAPWALNHMSRRLSQMLLMFLLATCLLAIIFVPQEMQTLRVVLATLGVGAASLGITCSTAQENELIPSIIRGRATGITGNFANIGGALASLMMILSIYSRPLPWIIYGVFAILSGLVVLLLPETRNQPLLDSIQDVENEGVNSLAAPQRSSVL

SEQ ID NO: 39 - RNA, 444 nt, Homo sapiens
  source 1..444: mol_type = mRNA
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 40 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 41 - RNA, 828 nt, Homo sapiens
  source 1..828: mol_type = mRNA
atgtcaacaattgggagttttgaaggattccaggctgtgtctctgaagcaagagggagatgaccaaccctctgagactgaccacctatcgatggaggaagaggacccgatgccaagacagatttcaaggcagtcaagtgtgaccgaatcaactctttaccccaatccttatcatcagccttatatctcacggaagtactttgctacacggccgggggccattgagactgccatggaagacttgaaaggtcacgtagctgagacttctggagagaccattcaaggcttctggctcttgacaaagatagaccactggaacaatgagaaggagagaattctactggtcacagacaagactctcttgatctgcaaatacgacttcatcatgctgagttgtgtgcagctgcagcggattcctctgagcgctgtctatcgcatctgcctgggcaagttcaccttccctgggatgtccctggacaagagacaaggagaaggccttaggatctactgggggagtccggaggagcagtctcttctgtcccgctggaacccatggtccactgaagttccttatgctactttcactgagcatcctatgaaatacaccagtgagaaattccttgaaatttgcaagttgtctgggttcatgtctaagcttgttccagctatccagaatgcccacaagaattcaactggatctggaagaggaaagaaactgatggtgttaactgaacccattttgattgagacctacacagggctgatgtcattcattggaaaccgcaacaaacttggctattcccttgcccgtgggagtattggtttttga

SEQ ID NO: 42 - protein, 275 aa, Homo sapiens
MSTIGSFEGFQAVSLKQEGDDQPSETDHLSMEEEDPMPRQISRQSSVTESTLYPNPYHQPYISRKYFATRPGAIETAMEDLKGHVAETSGETIQGFWLLTKIDHWNNEKERILLVTDKTLLICKYDFIMLSCVQLQRIPLSAVYRICLGKFTFPGMSLDKRQGEGLRIYWGSPEEQSLLSRWNPWSTEVPYATFTEHPMKYTSEKFLEICKLSGFMSKLVPAIQNAHKNSTGSGRGKKLMVLTEPILIETYTGLMSFIGNRNKLGYSLARGSIGF

SEQ ID NO: 43 - RNA, 19 nt, synthetic construct
  source 1..19: mol_type = other RNA
tactgattcatccatgttg

SEQ ID NO: 44 - RNA, 19 nt, synthetic construct
  source 1..19: mol_type = other RNA
caacatggatgaatcagta

SEQ ID NO: 45 - RNA, 19 nt, synthetic construct
  source 1..19: mol_type = other RNA
taaatgccaaggtcttcgc

SEQ ID NO: 46 - RNA, 19 nt, synthetic construct
  source 1..19: mol_type = other RNA
gcgaagaccttggcattta

SEQ ID NO: 47 - RNA, 19 nt, synthetic construct
  source 1..19: mol_type = other RNA
ttatctctgggcaaagttc

SEQ ID NO: 48 - RNA, 19 nt, synthetic construct
  source 1..19: mol_type = other RNA
gaactttgcccagagataa

SEQ ID NO: 49 - RNA, 19 nt, synthetic construct
  source 1..19: mol_type = other RNA
tagcagttaatgttgttgg

SEQ ID NO: 50 - RNA, 19 nt, synthetic construct
  source 1..19: mol_type = other RNA
ccaacaacattaactgcta

SEQ ID NO: 51 - RNA, 19 nt, synthetic construct
  source 1..19: mol_type = other RNA
tctcgcaccactctgaaga

SEQ ID NO: 52 - RNA, 19 nt, synthetic construct
  source 1..19: mol_type = other RNA
tcttcagagtggtgcgaga

SEQ ID NO: 53 - RNA, 19 nt, synthetic construct
  source 1..19: mol_type = other RNA
taatacgaacttctggatc

SEQ ID NO: 54 - RNA, 19 nt, synthetic construct
  source 1..19: mol_type = other RNA
gatccagaagttcgtatta

SEQ ID NO: 55 - RNA, 19 nt, synthetic construct
  source 1..19: mol_type = other RNA
cattcgtccatccatctgc

SEQ ID NO: 56 - RNA, 19 nt, synthetic construct
  source 1..19: mol_type = other RNA
gcagatggatggacgaatg

SEQ ID NO: 57 - RNA, 19 nt, synthetic construct
  source 1..19: mol_type = other RNA
aaccaaggtatttcttcgg

SEQ ID NO: 58 - RNA, 19 nt, synthetic construct
  source 1..19: mol_type = other RNA
ccgaagaaataccttggtt

SEQ ID NO: 59 - RNA, 19 nt, synthetic construct
  source 1..19: mol_type = other RNA
tgatccgtgagctatggcc

SEQ ID NO: 60 - RNA, 19 nt, synthetic construct
  source 1..19: mol_type = other RNA
ggccatagctcacggatca

SEQ ID NO: 61 - RNA, 19 nt, synthetic construct
  source 1..19: mol_type = other RNA
taagcagctagttctttgt

SEQ ID NO: 62 - RNA, 19 nt, synthetic construct
  source 1..19: mol_type = other RNA
acaaagaactagctgctta

SEQ ID NO: 63 - RNA, 19 nt, synthetic construct
  source 1..19: mol_type = other RNA
tagcaatagggttaactgc

SEQ ID NO: 64 - RNA, 19 nt, synthetic construct
  source 1..19: mol_type = other RNA
gcagttaaccctattgcta

SEQ ID NO: 65 - RNA, 19 nt, synthetic construct
  source 1..19: mol_type = other RNA
aaagtgaacatcattgaac

SEQ ID NO: 66 - RNA, 19 nt, synthetic construct
  source 1..19: mol_type = other RNA
gttcaatgatgttcacttt

SEQ ID NO: 67 - RNA, 19 nt, synthetic construct
  source 1..19: mol_type = other RNA
taggactacttctatctcc

SEQ ID NO: 68 - RNA, 19 nt, synthetic construct
  source 1..19: mol_type = other RNA
ggagatagaagtagtccta

SEQ ID NO: 69 - RNA, 19 nt, synthetic construct
  source 1..19: mol_type = other RNA
ttatagaaaacccaaatcc

SEQ ID NO: 70 - RNA, 19 nt, synthetic construct
  source 1..19: mol_type = other RNA
ggatttgggttttctataa

SEQ ID NO: 71 - RNA, 19 nt, synthetic construct
  source 1..19: mol_type = other RNA
ttgaatgaaacgacttctc

SEQ ID NO: 72 - RNA, 19 nt, synthetic construct
  source 1..19: mol_type = other RNA
gagaagtcgtttcattcaa

SEQ ID NO: 73 - RNA, 19 nt, synthetic construct
  source 1..19: mol_type = other RNA
tagacatccagataatcct

SEQ ID NO: 74 - RNA, 19 nt, synthetic construct
  source 1..19: mol_type = other RNA
aggattatctggatgtcta

SEQ ID NO: 75 - RNA, 19 nt, synthetic construct
  source 1..19: mol_type = other RNA
tacctctgcatttgcctac

SEQ ID NO: 76 - RNA, 19 nt, synthetic construct
  source 1..19: mol_type = other RNA
gtaggcaaatgcagaggta

SEQ ID NO: 77 - RNA, 19 nt, synthetic construct
  source 1..19: mol_type = other RNA
caagtaggcatacctgtac

SEQ ID NO: 78 - RNA, 19 nt, synthetic construct
  source 1..19: mol_type = other RNA
gtacaggtatgcctacttg

SEQ ID NO: 79 - RNA, 19 nt, synthetic construct
  source 1..19: mol_type = other RNA
gattgttacactgcctttg

SEQ ID NO: 80 - RNA, 19 nt, synthetic construct
  source 1..19: mol_type = other RNA
caaaggcagtgtaacaatc

SEQ ID NO: 81 - RNA, 19 nt, synthetic construct
  source 1..19: mol_type = other RNA
tcctaatatagtgtttgcc

SEQ ID NO: 82 - RNA, 19 nt, synthetic construct
  source 1..19: mol_type = other RNA
ggcaaacactatattagga

SEQ ID NO: 83 - RNA, 19 nt, synthetic construct
  source 1..19: mol_type = other RNA
taaatcaccagatgaactc

SEQ ID NO: 84 - RNA, 19 nt, synthetic construct
  source 1..19: mol_type = other RNA
gagttcatctggtgattta

SEQ ID NO: 85 - RNA, 19 nt, synthetic construct
  source 1..19: mol_type = other RNA
caaatctccgactatcttc

SEQ ID NO: 86 - RNA, 19 nt, synthetic construct
  source 1..19: mol_type = other RNA
gaagatagtcggagatttg

SEQ ID NO: 87 - RNA, 19 nt, synthetic construct
  source 1..19: mol_type = other RNA
taatttcccacatcagacg

SEQ ID NO: 88 - RNA, 19 nt, synthetic construct
  source 1..19: mol_type = other RNA
cgtctgatgtgggaaatta

SEQ ID NO: 89 - RNA, 19 nt, synthetic construct
  source 1..19: mol_type = other RNA
agaagcaacaactttgagc

SEQ ID NO: 90 - RNA, 19 nt, synthetic construct
  source 1..19: mol_type = other RNA
gctcaaagttgttgcttct

SEQ ID NO: 91 - RNA, 19 nt, synthetic construct
  source 1..19: mol_type = other RNA
atatccgtgacatcattgc

SEQ ID NO: 92 - RNA, 19 nt, synthetic construct
  source 1..19: mol_type = other RNA
gcaatgatgtcacggatat

SEQ ID NO: 93 - RNA, 19 nt, synthetic construct
  source 1..19: mol_type = other RNA
taatgcgttatgcaatcgt

SEQ ID NO: 94 - RNA, 19 nt, synthetic construct
  source 1..19: mol_type = other RNA
acgattgcataacgcatta

SEQ ID NO: 95 - RNA, 19 nt, synthetic construct
  source 1..19: mol_type = other RNA
ttaaggtgtatactccatc

SEQ ID NO: 96 - RNA, 19 nt, synthetic construct
  source 1..19: mol_type = other RNA
gatggagtatacaccttaa

SEQ ID NO: 97 - RNA, 19 nt, synthetic construct
  source 1..19: mol_type = other RNA
gctccacatagccatttgc

SEQ ID NO: 98 - RNA, 19 nt, synthetic construct
  source 1..19: mol_type = other RNA
gcaaatggctatgtggagc

SEQ ID NO: 99 - RNA, 19 nt, synthetic construct
  source 1..19: mol_type = other RNA
tcttattggtgctccgtgg

SEQ ID NO: 100 - RNA, 19 nt, synthetic construct
  source 1..19: mol_type = other RNA
ccacggagcaccaataaga

SEQ ID NO: 101 - RNA, 19 nt, synthetic construct
  source 1..19: mol_type = other RNA
tattaatgaccgtgtaacc

SEQ ID NO: 102 - RNA, 19 nt, synthetic construct
  source 1..19: mol_type = other RNA
ggttacacggtcattaata

SEQ ID NO: 103 - RNA, 19 nt, synthetic construct
  source 1..19: mol_type = other RNA
tgaaggtagaatcctatac

SEQ ID NO: 104 - RNA, 19 nt, synthetic construct
  source 1..19: mol_type = other RNA
gtataggattctaccttca

SEQ ID NO: 105 - RNA, 19 nt, synthetic construct
  source 1..19: mol_type = other RNA
tctgaagaacactcattcc

SEQ ID NO: 106 - RNA, 19 nt, synthetic construct
  source 1..19: mol_type = other RNA
ggaatgagtgttcttcaga

SEQ ID NO: 107 - RNA, 19 nt, synthetic construct
  source 1..19: mol_type = other RNA
aagccctacggcattcgcg

SEQ ID NO: 108 - RNA, 19 nt, synthetic construct
  source 1..19: mol_type = other RNA
cgcgaatgccgtagggctt

SEQ ID NO: 109 - RNA, 19 nt, synthetic construct
  source 1..19: mol_type = other RNA
ttcacttagactgcggatc

SEQ ID NO: 110 - RNA, 19 nt, synthetic construct
  source 1..19: mol_type = other RNA
gatccgcagtctaagtgaa

SEQ ID NO: 111 - RNA, 19 nt, synthetic construct
  source 1..19: mol_type = other RNA
attaatattatccacctcg

SEQ ID NO: 112 - RNA, 19 nt, synthetic construct
  source 1..19: mol_type = other RNA
cgaggtggataatattaat

SEQ ID NO: 113 - RNA, 19 nt, synthetic construct
  source 1..19: mol_type = other RNA
agaacctgtcacttgatcc

SEQ ID NO: 114 - RNA, 19 nt, synthetic construct
  source 1..19: mol_type = other RNA
ggatcaagtgacaggttct

SEQ ID NO: 115 - RNA, 19 nt, synthetic construct
  source 1..19: mol_type = other RNA
actgtaaacatcgcagacc

SEQ ID NO: 116 - RNA, 19 nt, synthetic construct
  source 1..19: mol_type = other RNA
ggtctgcgatgtttacagt

SEQ ID NO: 117 - RNA, 19 nt, synthetic construct
  source 1..19: mol_type = other RNA
atctgagccgcctcttctt

SEQ ID NO: 118 - RNA, 19 nt, synthetic construct
  source 1..19: mol_type = other RNA
aagaagaggcggctcagat

SEQ ID NO: 119 - RNA, 19 nt, synthetic construct
  source 1..19: mol_type = other RNA
tctgcgtcagcctcacaac

SEQ ID NO: 120 - RNA, 19 nt, synthetic construct
  source 1..19: mol_type = other RNA
gttgtgaggctgacgcaga

SEQ ID NO: 121 - RNA, 19 nt, synthetic construct
  source 1..19: mol_type = other RNA
tcttcgggaggctgttact

SEQ ID NO: 122 - RNA, 19 nt, synthetic construct
  source 1..19: mol_type = other RNA
agtaacagcctcccgaaga

SEQ ID NO: 123 - RNA, 19 nt, synthetic construct
  source 1..19: mol_type = other RNA
aatttcgatgtgtaactct

SEQ ID NO: 124 - RNA, 19 nt, synthetic construct
  source 1..19: mol_type = other RNA
agagttacacatcgaaatt

SEQ ID NO: 125 - RNA, 19 nt, synthetic construct
  source 1..19: mol_type = other RNA
tacatgttaatgagctctg

SEQ ID NO: 126 - RNA, 19 nt, synthetic construct
  source 1..19: mol_type = other RNA
cagagctcattaacatgta

SEQ ID NO: 127 - RNA, 19 nt, synthetic construct
  source 1..19: mol_type = other RNA
tctacttgcccaattgttg

SEQ ID NO: 128 - RNA, 19 nt, synthetic construct
  source 1..19: mol_type = other RNA
caacaattgggcaagtaga

SEQ ID NO: 129 - RNA, 19 nt, synthetic construct
  source 1..19: mol_type = other RNA
cagtctatcagagaggtcc

SEQ ID NO: 130 - RNA, 19 nt, synthetic construct
  source 1..19: mol_type = other RNA
ggacctctctgatagactg

SEQ ID NO: 131 - RNA, 19 nt, synthetic construct
  source 1..19: mol_type = other RNA
tctttaatcatcataacgc

SEQ ID NO: 132 - RNA, 19 nt, synthetic construct
  source 1..19: mol_type = other RNA
gcgttatgatgattaaaga

SEQ ID NO: 133 - RNA, 19 nt, synthetic construct
  source 1..19: mol_type = other RNA
actccgtatggccatgttt

SEQ ID NO: 134 - RNA, 19 nt, synthetic construct
  source 1..19: mol_type = other RNA
aaacatggccatacggagt

SEQ ID NO: 135 - RNA, 19 nt, synthetic construct
  source 1..19: mol_type = other RNA
atgaactctgtccaatttg

SEQ ID NO: 136 - RNA, 19 nt, synthetic construct
  source 1..19: mol_type = other RNA
caaattggacagagttcat

SEQ ID NO: 137 - RNA, 19 nt, synthetic construct
  source 1..19: mol_type = other RNA
tacggcttagcttcaacat

SEQ ID NO: 138 - RNA, 19 nt, synthetic construct
  source 1..19: mol_type = other RNA
atgttgaagctaagccgta

SEQ ID NO: 139 - RNA, 19 nt, synthetic construct
  source 1..19: mol_type = other RNA
ataggaaccacattgttgc

SEQ ID NO: 140 - RNA, 19 nt, synthetic construct
  source 1..19: mol_type = other RNA
gcaacaatgtggttcctat

SEQ ID NO: 141 - RNA, 19 nt, synthetic construct
  source 1..19: mol_type = other RNA
tagctcgatatcgctcttg

SEQ ID NO: 142 - RNA, 19 nt, synthetic construct
  source 1..19: mol_type = other RNA
caagagcgatatcgagcta

SEQ ID NO: 143 - RNA, 19 nt, synthetic construct
  source 1..19: mol_type = other RNA
tgttctgacagcactgcac

SEQ ID NO: 144 - RNA, 19 nt, synthetic construct
  source 1..19: mol_type = other RNA
gtgcagtgctgtcagaaca

SEQ ID NO: 145 - RNA, 19 nt, synthetic construct
  source 1..19: mol_type = other RNA
aaacaatgcggtaaatctc

SEQ ID NO: 146 - RNA, 19 nt, synthetic construct
  source 1..19: mol_type = other RNA
gagatttaccgcattgttt

SEQ ID NO: 147 - RNA, 19 nt, synthetic construct
  source 1..19: mol_type = other RNA
gattccacagctcttttac

SEQ ID NO: 148 - RNA, 19 nt, synthetic construct
  source 1..19: mol_type = other RNA
gtaaaagagctgtggaatc

SEQ ID NO: 149 - RNA, 19 nt, synthetic construct
  source 1..19: mol_type = other RNA
ctactaaacagatttcctt

SEQ ID NO: 150 - RNA, 19 nt, synthetic construct
  source 1..19: mol_type = other RNA
aaggaaatctgtttagtag

SEQ ID NO: 151 - RNA, 19 nt, synthetic construct
  source 1..19: mol_type = other RNA
ttctccaatgaatcgctta

SEQ ID NO: 152 - RNA, 19 nt, synthetic construct
  source 1..19: mol_type = other RNA
taagcgattcattggagaa

SEQ ID NO: 153 - RNA, 19 nt, synthetic construct
  source 1..19: mol_type = other RNA
aataacaaacccatctgcc

SEQ ID NO: 154 - RNA, 19 nt, synthetic construct
  source 1..19: mol_type = other RNA
ggcagatgggtttgttatt

SEQ ID NO: 155 - RNA, 19 nt, synthetic construct
  source 1..19: mol_type = other RNA
ttcaatgggaaacttatcc

SEQ ID NO: 156 - RNA, 19 nt, synthetic construct
  source 1..19: mol_type = other RNA
ggataagtttcccattgaa

SEQ ID NO: 157 - RNA, 18 nt, synthetic construct
  source 1..18: mol_type = other RNA
taaggaggagttccgtcg

SEQ ID NO: 158 - RNA, 18 nt, synthetic construct
  source 1..18: mol_type = other RNA
cgacggaactcctcctta

SEQ ID NO: 159 - RNA, 19 nt, synthetic construct
  source 1..19: mol_type = other RNA
tctcttcacagctacgtcc

SEQ ID NO: 160 - RNA, 19 nt, synthetic construct
  source 1..19: mol_type = other RNA
ggacgtagctgtgaagaga

SEQ ID NO: 161 - RNA, 19 nt, synthetic construct
  source 1..19: mol_type = other RNA
acatctttgaggttattgt

SEQ ID NO: 162 - RNA, 19 nt, synthetic construct
  source 1..19: mol_type = other RNA
acaataacctcaaagatgt

SEQ ID NO: 163 - RNA, 19 nt, synthetic construct
  source 1..19: mol_type = other RNA
tttccgacaaccaaatagg

SEQ ID NO: 164 - RNA, 19 nt, synthetic construct
  source 1..19: mol_type = other RNA
cctatttggttgtcggaaa

SEQ ID NO: 165 - RNA, 19 nt, synthetic construct
  source 1..19: mol_type = other RNA
aaacgcaaagggaacaacg

SEQ ID NO: 166 - RNA, 19 nt, synthetic construct
  source 1..19: mol_type = other RNA
cgttgttccctttgcgttt

SEQ ID NO: 167 - RNA, 19 nt, synthetic construct
  source 1..19: mol_type = other RNA
atccgctacctccactctc

SEQ ID NO: 168 - RNA, 19 nt, synthetic construct
  source 1..19: mol_type = other RNA
gagagtggaggtagcggat

SEQ ID NO: 169 - RNA, 19 nt, synthetic construct
  source 1..19: mol_type = other RNA
actaactcaaaggctatgg

SEQ ID NO: 170 - RNA, 19 nt, synthetic construct
  source 1..19: mol_type = other RNA
ccatagcctttgagttagt

SEQ ID NO: 171 - RNA, 19 nt, synthetic construct
  source 1..19: mol_type = other RNA
ttatggaaggaattagttc

SEQ ID NO: 172 - RNA, 19 nt, synthetic construct
  source 1..19: mol_type = other RNA
gaactaattccttccataa

SEQ ID NO: 173 - RNA, 19 nt, synthetic construct
  source 1..19: mol_type = other RNA
gaacccagcagcgatgatc

SEQ ID NO: 174 - RNA, 19 nt, synthetic construct
  source 1..19: mol_type = other RNA
gatcatcgctgctgggttc

SEQ ID NO: 175 - RNA, 19 nt, synthetic construct
  source 1..19: mol_type = other RNA
tatgtccaatactagcagc

SEQ ID NO: 176 - RNA, 19 nt, synthetic construct
  source 1..19: mol_type = other RNA
gctgctagtattggacata

SEQ ID NO: 177 - RNA, 19 nt, synthetic construct
  source 1..19: mol_type = other RNA
gaattggtgagttatccac

SEQ ID NO: 178 - RNA, 19 nt, synthetic construct
  source 1..19: mol_type = other RNA
gtggataactcaccaattc

SEQ ID NO: 179 - RNA, 19 nt, synthetic construct
  source 1..19: mol_type = other RNA
aagatttggtgtctatttc

SEQ ID NO: 180 - RNA, 19 nt, synthetic construct
  source 1..19: mol_type = other RNA
gaaatagacaccaaatctt

SEQ ID NO: 181 - RNA, 19 nt, synthetic construct
  source 1..19: mol_type = other RNA
acaaattcctcctcagttg

SEQ ID NO: 182 - RNA, 19 nt, synthetic construct
  source 1..19: mol_type = other RNA
caactgaggaggaatttgt

SEQ ID NO: 183 - RNA, 19 nt, synthetic construct
  source 1..19: mol_type = other RNA
cctttctgaacacatgcac

SEQ ID NO: 184 - RNA, 19 nt, synthetic construct
  source 1..19: mol_type = other RNA
gtgcatgtgttcagaaagg

SEQ ID NO: 185 - RNA, 19 nt, synthetic construct
  source 1..19: mol_type = other RNA
ataccacctctgcatgctc

SEQ ID NO: 186 - RNA, 19 nt, synthetic construct
  source 1..19: mol_type = other RNA
gagcatgcagaggtggtat

SEQ ID NO: 187 - RNA, 19 nt, synthetic construct
  source 1..19: mol_type = other RNA
ttatcttgaactgctatcc

SEQ ID NO: 188 - RNA, 19 nt, synthetic construct
  source 1..19: mol_type = other RNA
ggatagcagttcaagataa

SEQ ID NO: 189 - RNA, 19 nt, synthetic construct
  source 1..19: mol_type = other RNA
tatcccattgttacagact

SEQ ID NO: 190 - RNA, 19 nt, synthetic construct
  source 1..19: mol_type = other RNA
agtctgtaacaatgggata

SEQ ID NO: 191 - RNA, 19 nt, synthetic construct
  source 1..19: mol_type = other RNA
acttccgtgagatataagg

SEQ ID NO: 192 - RNA, 19 nt, synthetic construct
  source 1..19: mol_type = other RNA
ccttatatctcacggaagt

SEQ ID NO: 193 - RNA, 19 nt, synthetic construct
  source 1..19: mol_type = other RNA
ttcctctcacaatatcgac

SEQ ID NO: 194 - RNA, 19 nt, synthetic construct
  source 1..19: mol_type = other RNA
gtcgatattgtgagaggaa